(19) United States
(12) Patent Application Publication
(10) Pub. No.: US 2026/0297169 A1
(43) Pub. Date: Oct. 1, 2026

(54) ANTI-ALLERGEN ANTIBODIES AND USES THEREOF

(71) Applicant: MABYLON AG, Schlieren (CH)

(72) Inventors: Dimitri BIELI, Basel (CH); Natascha WUILLEMIN, Zürich (CH); Tudor ILCA, Zürich (CH); Chiara ARENA, Zürich (CH); Niccolò PENGO, Adliswil (CH); Tiziana SONATI, Zürich (CH); Alcide BARBERIS, Schindellegi (CH); Pascal GASSER, Zürich (CH); Douglas PHILLIPS, Baden (CH); Yvonne STARK, Zürich (CH)

(21) Appl. No.: 19/489,402

(22) PCT Filed: Jun. 6, 2024
(86) PCT No.: PCT/EP2024/065665

§ 371 Date: Dec. 2, 2025

(30) Foreign Application Priority Data
Jun. 7, 2023 (WO) PCT/EP2023/065193

(51) Int. Cl. C07K 16/16; A61K 39/00; A61K 45/06; A61P 37/08

(52) U.S. Cl. CPC C07K 16/16; A61K 45/06; A61P 37/08; A61K 2039/507; C07K 2317/21; C07K 2317/31; C07K 2317/34; C07K 2317/52; C07K 2317/565; C07K 2317/567; C07K 2317/622

(57) ABSTRACT

The present invention provides antibodies binding to birch pollen allergens, in particular to Bet v 1 allergens, and also binding to Bet v 1 homologous allergens. The present invention also provides compositions and kits comprising distinct antibodies binding to distinct, non-overlapping epitopes of Betv 1 and Betv 1 homologous proteins. In addition, the present invention also relates to the use of such antibodies, compositions and kits, e.g. for preventing or treating tree pollen allergies as well as food-related allergies.

The present invention relates to antibodies binding to birch pollen allergens and related tree pollen and food allergens, in particular binding to Bet v 1 and Bet v 1 homologous allergens/proteins. The present invention also relates to compositions and kits comprising distinct antibodies binding to distinct, non-overlapping epitopes of Bet v 1 and Bet v 1 homologous proteins. In addition, the present invention also relates to the use of such antibodies, compositions and kits, e.g. for preventing or treating tree pollen and food allergies, in particular birch pollen and related tree pollen allergies as well as food-related allergies, such as e.g. apple, hazelnut and walnut allergies.

Allergies are conditions caused by hypersensitivity of the immune system. Allergen encounter results in the production of allergen-binding immunoglobulin E (IgE) antibodies, which are pre-bound on FcεRI receptors on mast cells and basophils, where they trigger the release of inflammatory compounds, such as histamine, leukotriene and lipid mediators.

Type I allergy or immediate hypersensitivity is the consequence of the interaction between an allergen and immunoglobulin E (IgE) antibodies, which are attached to mast cells. Allergic symptoms, including allergic asthma, rhino-conjunctivitis, and atopic dermatitis, significantly affect human health and a quality of life. It is estimated that 300-400 million people in the world is affected by type 1 allergies, causing 120 billion Euros in health care costs, sick leaves, and economic losses (Calderon et al., 2012, Clin. Transl. Allergy 2: 20). These symptoms are elicited by environmental aeroallergens such as pollen or house dust mites. Birch pollen is widespread in Europe, North America, Russia, and northern Japan. More than 100 million patients suffer from allergy to birch pollen (Moverare et al., 2005, Int. Arch. Allergy Immunol. 136, 33-38). The major birch pollen allergen is Bet v 1.

The Bet v 1 allergen is a member of the plant pathogenesis related proteins family PR-10. Bet v 1 homologs are also included among various plant-derived food allergens such as apple Mal d 1, and hazelnut Cor a 1. The patients having birch pollen allergy usually suffer from other related tree pollen allergies, such as hazel, alder, beech, oak pollen, and also often suffer from secondary pollen food syndrome, which negatively impacts on their health-related quality of life (Popescu, 2015, World J. Methodol. 5, 31-50).

Allergen immunotherapy (AIT) is based on the repeated administration of disease-eliciting allergens to modulate the allergic immune response. During AIT, allergen-specific immunoglobulin G, particularly IgG4, is induced. IgG4 competes with IgE to bind the allergen, leading to the inhibition of the IgE-mediated hypersensitive mechanisms to alleviate allergic symptoms (Shamji et al., 2012, Allergy 67, 217-226). Bet v 1-specific IgG4 developed by AIT competes with IgE for partly identical or largely overlapping epitopes (Groh et al., 2017, Clin. Exp. Allergy 47, 693-703).

Recently, antibodies against birch pollen allergens emerged as another promising option for treating birch pollen allergy. For example, Atanasio et al. (J. Allergy Clin. Immunol. 2020; vol. 149, 1, p. 200-211) describes that a combination of mAbs targeting Bet v 1 can prevent the birch allergic response. According to that study, Bet v 1-specific mAbs, REGN5713, REGN5714, and REGN5715, derived from immunization of humanized mice, bind with high affinity and noncompetitively to Bet v 1. It has been shown that a cocktail of all 3 antibodies, REGN5713/14/15, blocks IgE binding to Bet v 1 and inhibits Bet v 1—and birch pollen extract—induced basophil activation ex vivo and mast cell degranulation in vivo.

Due to the high cross-reactivity observed in birch allergic patients, it would be beneficial to target—in a birch pollen-targeting antibody approach—as many Bet v 1 homologues proteins (PR-10 proteins) as possible.

In view of the above, it is the object of the present invention to provide an improved antibody against birch pollen allergens, in particular an antibody which targets the Bet v 1 allergen and also is cross-reactive with several Bet v 1 homologous allergens/proteins. It is also an object of the present invention to provide a composition comprising at least two distinct potent antibodies binding to distinct, non-overlapping epitopes on the major birch allergen Bet v 1, and optionally binding to at least one further Bet v 1 homologous allergen/protein.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%, for example, x±5%, or x±7%, or x±10%, or x 12%, or x±15%, or x±20%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. In some embodiments, the subject or patient is a human.

Doses are often expressed in relation to the bodyweight. Thus, a dose which is expressed as [g, mg, or other unit]/kg (or g, mg etc.) usually refers to [g, mg, or other unit]“per kg (or g, mg etc.) bodyweight”, even if the term “bodyweight” is not explicitly mentioned.

The term “binding” and similar reference usually means “specifically binding”, which does not encompass non-specific sticking. In particular, specific binding of an antibody means that the antibody recognizes its target antigen and binds its target with greater affinity (or at lower antibody concentrations, e.g. EC50) than it does to a structurally different antigen and/or to an antigen with a modified or mutated sequence. Thereby, a “greater” affinity may be at least 2 fold, 3 fold, 4 fold, 5 fold, 10 fold, 15 fold, 20 fold, 25 fold, 50 fold, 75 fold, 100 fold 150 fold, 200 fold, 500 fold, 750 fold, 1,000 fold, 1,500 fold, 2,000 fold, 5,000 fold, 7,500 fold, 10,000 fold or even higher affinity as compared to the binding to a control antigen. In some instances, antibody-binding to the control antigen may be undetectable (below detection threshold), while antibody-binding to the specific antigen may be well detected/determined.

As used herein, the term “antibody” encompasses various forms of antibodies including, without being limited to, whole antibodies, antibody fragments (such as antigen binding fragments), human antibodies, chimeric antibodies, humanized antibodies, recombinant antibodies and genetically engineered antibodies (e.g., variant or mutant antibodies) as long as the characteristic properties according to the invention are retained. In some embodiments, the antibody is a human antibody. In some embodiments, the antibody is a monoclonal antibody. For example, the antibody may be a human monoclonal antibody.

As described above, the term “antibody” generally also includes antibody fragments. Fragments of the antibodies may retain the antigen-binding activity of the antibodies. Such fragments are referred to as “antigen-binding fragments”. Antigen-binding fragments include, but are not limited to, single chain antibodies, Fab, Fab′, F(ab′)2, Fv or scFv. Fragments of the antibodies can be obtained from the antibodies by methods that include digestion with enzymes, such as pepsin or papain, and/or by cleavage of disulfide bonds by chemical reduction. Alternatively, fragments of the antibodies can be obtained by recombinant means, for example by cloning and expressing a part (fragment) of the sequences of the heavy and/or light chain. The invention also encompasses single-chain Fv fragments (scFv) derived from the heavy and light chains of an antibody of the invention. For example, the invention includes a scFv comprising the CDRs from an antibody of the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker. Antibody fragments of the invention may be contained in a variety of structures known to the person skilled in the art. In addition, the sequences of the invention may be a component of multispecific molecules in which the sequences of the invention target the epitopes of the invention and other regions of the molecule bind to other targets. Although the specification, including the claims, may, in some places, refer explicitly to antigen binding fragment(s), antibody fragment(s), variant(s) and/or derivative(s) of antibodies, it is understood that the term “antibody” includes all categories of antibodies, namely, antigen binding fragment(s), antibody fragment(s), variant(s) and derivative(s) of antibodies.

Human antibodies are well-known in the state of the art (van Dijk, M. A., and van de Winkel, J. G., Curr. Opin. Chem. Biot. 5 (2001) 368-374). Human antibodies can also be produced in transgenic animals (e.g., mice or chicken) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biot. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). As used herein, the expression “human antibodies” includes non-naturally occurring sequence variants of human antibodies, which are usually obtained by introducing one or more mutations in the (naturally occurring) human antibodies. Such mutations include one or more mutations in a CDR or in a framework region, as well as Fc modifications (e.g., as known in the art for specific functionalities).

As used herein, the term “variable region” (variable region of a light chain (V_{L}), variable region of a heavy chain (V_{H})) denotes each of the pair of light and heavy chains which is involved directly in binding the antibody to the antigen.

Antibodies of the invention can be of any isotype (e.g., IgA, IgG, IgM i.e. an α, γ or μ heavy chain). Preferably, the antibody is of the IgG type or the IgA type. Within the IgG isotype, antibodies may be IgG1, IgG2, IgG3 or IgG4 subclass, preferably IgG1 or IgG4. Antibodies of the invention may have a κ or a λ light chain.

Antibodies according to the present invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies according to the present invention may be immunogenic in human and/or in non-human (or heterologous) hosts e.g., in mice. For example, the antibodies may have an idiotope that is immunogenic in non-human hosts, but not in a human host. Antibodies of the invention for human use include those that cannot be easily isolated from hosts such as mice, goats, rabbits, rats, non-primate mammals, etc. and cannot generally be obtained by humanization or from xeno-mice.

As used herein, the term “antigen” refers to any structural substance which serves as a target for the receptors of an adaptive immune response, in particular as a target for antibodies, T cell receptors, and/or B cell receptors. An “epitope”, also known as “antigenic determinant”, is the part (or fragment) of an antigen that is recognized by the immune system, in particular by antibodies, T cell receptors, and/or B cell receptors. Thus, one antigen has at least one epitope, i.e. a single antigen has one or more epitopes. An antigen may be (i) a peptide, a polypeptide, or a protein, (ii) a polysaccharide, (iii) a lipid, (iv) a lipoprotein or a lipopeptide, (v) a glycolipid, (vi) a nucleic acid, or (vii) a small molecule drug or a toxin. Thus, an antigen may be a peptide, a protein, a polysaccharide, a lipid, a combination thereof including lipoproteins and glycolipids, a nucleic acid (e.g. DNA, siRNA, shRNA, antisense oligonucleotides, decoy DNA, plasmid), or a small molecule drug (e.g. cyclosporine A, paclitaxel, doxorubicin, methotrexate, 5-aminolevulinic acid), or any combination thereof. Preferably, the antigen is selected from (i) a peptide, a polypeptide, or a protein, (ii) a polysaccharide, (iii) a lipid, (iv) a lipoprotein or a lipopeptide and (v) a glycolipid; more preferably, the antigen is a peptide, a polypeptide, or a protein.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

As used herein (i.e. throughout the present specification), the term “sequence variant” refers to any alteration in comparison to a reference sequence. The term “sequence variant” includes nucleotide sequence variants and amino acid sequence variants. Preferably, a reference sequence is any of the sequences listed in the “Table of Sequences and SEQ ID Numbers” (Sequence listing), i.e. SEQ ID NO: 1 to SEQ ID NO: 64. In particular, a sequence variant shares (over the whole length of the sequence) at least 70% or at least 75%, preferably at least 80% or at least 85%, more preferably at least 90% or at least 93%, even more preferably at least 95% or at least 96%, still more preferably at least 97% or at least 98%, particularly preferably at least 99% sequence identity with its reference sequence. In some embodiments, the sequence variant shares at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity. Thereby, the higher the %-identity of a sequence variant, the more it is preferred. For example, a sequence variant having at least 84% sequence identity with a reference sequence is more preferred than a sequence variant having at least 75% sequence identity, but less than 84% sequence identity, with a reference sequence. In some embodiments, the sequence variant maintains the (biological) function of the reference sequence. For example, sequence variants relating to antibodies of the invention preferably maintain the specific binding to Bet v 1 and/or a Bet v 1 homologous protein, such as e.g. Cor a 1, Aln g 1, Fag s 1, Que a 1, Cas s 1, Ost c 1, Car b1, Mal d 1, Jug r 5).

Sequence identity may be calculated as described below. Usually a sequence variant may preserve the specific function of the reference sequence. In some embodiments, an amino acid sequence variant has an altered sequence in which one or more (e.g., 1, 2, 3, 4, 5, 6, 7, 8, 9, 10 or more) of the amino acids in the reference sequence is deleted or substituted, or one or more (e.g., 1, 2, 3, 4, 5, 6, 7, 8, 9, 10 or more) amino acids are inserted into or added to the sequence of the reference amino acid sequence. As a result of the alterations, the amino acid sequence variant has an amino acid sequence which is at least 70% or at least 75%, preferably at least 80% or at least 85%, more preferably at least 90% or at least 93%, even more preferably at least 95% or at least 96%, still more preferably at least 97% or at least 98%, particularly preferably at least 99% identical to the reference sequence. For example, variant sequences which are at least 90% identical have no more than 10 alterations, i.e., any combination of deletions, insertions or substitutions, per 100 amino acids of the reference sequence. The same, of course, also applies similarly to nucleic acid sequences.

The “% identity” of the sequence variant is usually determined with respect to the reference sequence. It is usually calculated with regard to the full length of the reference sequence (i.e. the sequence recited in the application). Percentage identity, as referred to herein, can be determined, for example, by methods known in the art, such as BLAST using the default parameters specified by the NCBI (the National Center for Biotechnology Information; http://www.ncbi.nlm.nih.gov/) [Blosum 62 matrix; gap open penalty=11 and gap extension penalty=1].

In general, while it is possible to have non-conservative amino acid substitutions, the substitutions are preferably conservative amino acid substitutions, wherein the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, cysteine, and glycine, with another.

“Bet v 1 allergens or Bet v 1 proteins”, as used herein, encompasses all known isoallergens and variants of Bet v 1, including e.g. Bet v 1.0101, Bet v 1.0102, Bet v 1.0103, Bet v 1.0104, Bet v 1.0105, Bet v 1.0106, Bet v 1.0107, Bet v 1.0108, Bet v 1.0109, Bet v 1.0110, Bet v 1.0111, Bet v 1.0112, Bet v 1.0113, Bet v 1.0114, Bet v 1.0115, Bet v 1.0115, Bet v 1.0116, Bet v 1.0117, Bet v 1.0118, Bet v 1.0119, Bet v 1.0201, Bet v 1.0202, Bet v 1.0203, Bet v 1.0204, Bet v 1.0205, Bet v 1.0206, Bet v 1.0207, Bet v 1.0301, and also encompasses all known mutants of Bet v 1, such as e.g. Bet v 1 N160A.

Allergens or proteins of the Bet v 1 family, belonging to the class 10 of the pathogenesis related protein (PR 10) family, encompass Bet v 1 (European white birch; “https://www.meduniwien.ac.at/allfam/search.php?searchtext=Betula+verrcosa+%28Betula+pendula%29” \o “List all allergens from Betula verrucosa (Betula pendula)”), and also encompass “Bet v 1 homologous allergens”. Examples of “Bet v 1 homologous allergens” include e.g. Act c 8 (Actinidia chinensis, Gold kiwi fruit; “https://www.meduniwien.ac.at/allfam/search.php?searchtext=Actinidia+deliciosa” \o “List all allergens from Actinidia deliciosa”), Act d 8 (Actinidia deliciosa, Green Kiwi fruit), Ain g 1 (Alnus glutinosa, European alder; “https//www.meduniwien.ac.at/allfam/search.php?searchtext=Alnus+glutinosa” \o “List all allergens from Alnus glutinosa”), Api g 1 (Apium graveolens, Celery; “https//www.meduniwien.ac.at/allfam/search.php?searchtext=Apium+graveolens” \o “List all allergens from Apium graveolens”), Ara h 8 (Arachis hypogaea, Peanut; “https//www.meduniwien.ac.at/allfam/search.php?searchtext=Arachis+hypogaea” \o “List all allergens from Arachis hypogaea”), Bet pl PR-10 (Betula platyphylla, Asian white birch; “https//www.meduniwien.ac.at/allfam/search.php?searchtext=Betula+platyphylla” \o “List all allergens from Betula platyphylla”), Can s 5 (Cannabis sativa, Indian hemp), Car b 1 (Carpinus betulus, European hornbeam; “https://www.meduniwien.ac.at/allfam/search.php?searchtext=Carpinus+betulus” \o “List all allergens from Carpinus betulus”), Cas s 1 (Castanea sativa, Sweet chestnut; “https//www.meduniwien.ac.at/allfam/search.php?searchtext=Castanea+sativa” \o “List all allergens from Castanea sativa”), Cor a 1 (Corylus avellana, European hazel; “https://www.meduniwien.ac.at/allfam/search.php?searchtext=Corylus+avellana” \o “List all allergens from Corylus avellana”), Dau c 1 (Daucus carota, Carrot; “https//www.meduniwien.ac.at/allfam/search.php?searchtext=Daucus+carota” \o “List all allergens from Daucus carota”), Fag s 1 (Fagus sy/vatica, European beech; “https//www.meduniwien.ac.at/allfam/search.php?searchtext=Fagus+sylvatica” \o “List all allergens from Fagus sylvatica”), Fra a 1 (Fragaria ananassa, Strawberry; “https://www.meduniwien.ac.at/allfam/search.php?searchtext=Fragaria+ananassa” \o “List all allergens from Fragaria ananassa”), Gly m 4 (Glycine max, Soybean; “https://www.meduniwien.ac.at/allfam/search.php?searchtext=Glycine+max” \o “List all allergens from Glycine max”), Jug r 5 (Juglans regia, English walnut; “https//www.meduniwien.ac.at/allfam/search.php?searchtext=Juglans+regia” \o “List all allergens from Juglans regia”), Mal d 1 (Malus domestica, Apple; “https//www.meduniwien.ac.at/allfam/search.php?searchtext=Malus+domestica” \o “List all allergens from Malus domestica”), Mor a PR-10 (Morus alba, White mulberry; “https//www.meduniwien.ac.at/allfam/search.php?searchtext=Morus+alba” \o “List all allergens from Morus alba”), Mor b PR-10 (Morus bombycis, Chinese mulberry; “https//www.meduniwien.ac.at/allfam/search.php?searchtext=Morus+bombycis” \o “List all allergens from Morus bombycis”), Ost c 1 (Ostrya carpinifolia, European hop hornbeam; “https//www.meduniwien.ac.at/allfam/search.php?searchtext=Ostrya+carpinifolia” \o “List all allergens from Ostrya carpinifolia”), Prunus armeniaca, (Prunus armeniaca, Apricot; “https//www.meduniwien.ac.at/allfam/search.php?searchtext=Prunus+armeniaca” \o “List all allergens from Prunus armeniaca”), Pru av 1 (Prunus avium, Sweet cherry; “https://www.meduniwien.ac.at/allfam/search.php?searchtext=Prunus+avium” \o “List all allergens from Prunus avium”), Pru p 1 (Prunus persica, Peach; “https://www.meduniwien.ac.at/allfam/search.php?searchtext=Prunus+persica” \o “List all allergens from Prunus persica”), Pyrus communis, (Pyrus communis, Pear; “https//www.meduniwien.ac.at/allfam/search.php?searchtext=Pyrus+communis” \o “List all allergens from Pyrus communis”), Que a 1 (Quercus alba, White oak; “https//www.meduniwien.ac.at/allfam/search.php?searchtext=Quercus+alba” \o “List all allergens from Quercus alba”), Que ac 1 (Quercus acutissima, Sawtooth oak), Que i 1 (Quercus ilex, holly oak), Que m 1 (Quercus mongolica, Mongolian oak), Rub i 1 (Rubus idaeus, Red raspberry; “https//www.meduniwien.ac.at/allfa/search.php?searchtext=Rubus+idaeus” \o “List all allergens from Rubus idaeus”), Sola l 4 (Solanum lycopersicum, Tomato; “https://www.meduniwien.ac.at/allfam/search.php?searchtext=Solanum+lycopersicum” \o “List all allergens from Solanum lycopersicum”), Vigna radiata, (Vigna radiata, Mung bean; “https://www.meduniwien.ac.at/allfam/search.php?searchtext=Vigna+radiata” \o “List all allergens from Vigna radiata”), Vig r 6 (Vigna radiata, Mung bean; “https://www.meduniwien.ac.at/allfam/search.php?searchtext=Vigna+radiata” \o “List all allergens from Vigna radiata”). The contents of the indicated websites with respect to the allergens are incorporated herein by reference.

“Bet v 1 homologous allergens” also encompass all known isoallergens and variants of the above listed allergens, such as e.g. Aln g 1.0101, Car b 1.1A, Car b1.2, Car b 1.0101-Car b 1.0113, Car b 1.0201, Car b 1.301, Car b 1.302, Cas s 1.0101, Cor a 1.0101, Cor a 1.0102, Cor a 1.0103, Cor a 1.0104, Cor a 1.0201, Cor a 1.0301, Cor a 1.0401, Cor a 1.0402, Cor a 1.0403, Cor a 1.0404, Fag s 1.0101, Ost c 1.0101, Que a 1.0101, Que a 1.0201, Que a 1.0301, Que a 1.0401, Mal d 1.0101 to Mal d 1.0109, Mal d 1.0201 to Mal d 1.0208, Mal d 1.0301 to Mal d 1.0304, Mal d 1.0401 to Mal d 1.0403, Jug r 5.0101, Pru p 1.01 and Pru p 1.02 etc.

Accordingly, in the present specification, “birch pollen related allergies” encompass any allergy elicited by any of the above listed Bet v 1 homologous allergens or isoallergens.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

### Antibodies and Antigen-Binding Fragments Thereof.

In a first aspect the present invention provides an (isolated) antibody, or an antigen-binding fragment thereof, which (specifically) binds to Bet v 1 (Betula verrucose allergen 1). Bet v 1 is a major birch pollen allergen, which is recognized by serum IgE from more than 95% of patients with birch pollen hypersensitivity. Bet v 1 is a protein of approximately 17 kDa. A number of Bet v 1 isoforms are described (Bet v 1.0101, Bet v 1.0102, Bet v 1.0103 etc., supra). Accordingly, the antibody, or the antigen-binding fragment thereof, of the present invention binds in particular to a polypeptide or protein having an amino acid sequence according to SEQ ID NO: 52, and/or to a variant of a polypeptide or protein having an amino acid sequence according to SEQ ID NO: 52, as defined above.

Preferably, the (isolated) antibody, or an antigen-binding fragment thereof, which (specifically) binds to Bet v 1, further binds (specifically) to a Bet v 1 homologous allergen. The Bet v 1 homologous allergen may be another tree pollen allergen. The Bet v 1 homologous tree pollen allergen may be selected from the group consisting of Cor a 1 (Corylus avellana allergen 1), Aln g 1 (Alnus glutinosa allergen 1), Fag s 1 (Fagus sylvatica allergen 1), Que a 1 (Quercus alba allergen 1), Cas s 1 Castania sativa allergen 1), Ost c 1 Ostrya carpinifolia allergen 1), and/or Car b1 (Carbinus betulus allergen 1). Thus, preferably, the antibody, or the antigen-binding fragment thereof, of the invention binds (specifically) (i) to Bet v 1, and also binds (specifically) (ii) to at least one Bet v 1 homologous protein, such as e.g. Cor a 1, Aln g 1, Fag s 1, Que a 1, Cas s 1, Ost c 1, Car b1. Accordingly, the antibody, or the antigen-binding fragment thereof, of the present invention binds in particular to a polypeptide or protein having an amino acid sequence according to SEQ ID NO: 52, and/or to a variant of a polypeptide or protein having an amino acid sequence according to SEQ ID NO: 52, as defined above, and also preferably binds to a polypeptide or protein having an amino acid sequence according to any one of SEQ ID NOs 53-64, and/or to a variant of a polypeptide or protein having an amino acid sequence according to any one of SEQ ID NOs 53-64.

Moreover, the (isolated) antibody, or an antigen-binding fragment thereof, which (specifically) binds to Bet v 1, may further bind (specifically) to a Bet v 1 homologous food-related allergen. The food-related allergen may be selected from any of the Bet v 1 homologous allergens listed above, and is preferably selected from Mal d 1 (Malus domestica allergen 1), Cor a 1 (Corylus avellana allergen 1), Jug r 5 (Juglans regia allergen 5) and Pru p 1 (Prunus persica allergen 1; “https://www.meduniwien.ac.at/allfam/search.php?searchtext=Prunus+persica” \o “List all allergens from Prunus persica”).

The above tree pollen allergens, in particular Bet v 1, Cor a 1, Aln g 1, Fag s 1, Que a 1, Cas s 1, Ost c 1, and/or Car b1, and the above food-related allergens (e.g. Mal d 1, Cor a 1, Jug r 5, Pru p 1) may be of natural origin, recombinantly expressed or synthetic peptides.

Standard methods to assess binding of the antibody according to the present invention, or the antigen-binding fragment thereof, are known to those skilled in the art and include, for example, ELISA (enzyme-linked immunosorbent assay). Thereby, the relative affinities of antibody binding may be determined by measuring the concentration of the antibody (EC₅₀) required to achieve 50% maximal binding at saturation. A specific example of an ELISA, which may be used to assess binding of an antibody, is described in the example section of this specification.

In general, the antibody, or an antigen-binding fragment thereof, according to the present invention, may comprise (at least) three complementarity determining regions (CDRs) on a heavy chain and (at least) three CDRs on a light chain. In general, complementarity determining regions (CDRs) are the hypervariable regions present in heavy chain variable domains and light chain variable domains, respectively. Typically, the CDRs of a heavy chain and the connected light chain of an antibody together form the antigen receptor. Usually, the three CDRs (CDR1, CDR2, and CDR3) are arranged non-consecutively in the variable domain. Since antigen receptors are typically composed of two variable domains (on two different polypeptide chains, i.e. heavy and light chain: heavy chain variable region (VH) and light chain variable region (VL)), there are typically six CDRs for each antigen receptor (heavy chain: CDRH1, CDRH2, and CDRH3; light chain: CDRL1, CDRL2, and CDRL3). For example, a classical IgG antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a variable region (or each variable region, respectively) may be composed of four framework regions, separated by three CDR's.

The sequences of the heavy chains and light chains of exemplary antibodies of the invention, comprising three different CDRs on the heavy chain and three different CDRs on the light chain were determined. The CDR amino acid sequences of the CDR1 of the heavy chain (CDRH1), the CDR2 of the heavy chain (CDRH2), the CDR3 of the heavy chain (CDRH3), the CDR1 of the light chain (CDRL1), the CDR2 of the light chain (CDRL2) and the CDR3 of the light chain (CDRL3) of exemplary antibodies 4E1, 25-3D3 and 17B10, are shown in Table 1 below.

The CDRs, in particular the three different CDRs (CDR1, CDR2 and CDR3) on the heavy chain and three different CDRs (CDR1, CDR2 and CDR3) on the light chain, as identified by the present invention may be grafted on any variable framework region, in particular any variable human framework region, without abrogating their specificity.

The human variable framework regions of the heavy chain (VH) may be retrieved from the website: “https://www.imgt.org/genedb/resultPage.action;jsessionid=49EB34C22C79EAC51862C76108963216?gene.id.species=Homo+sapiens&molComponent=IG&geneTypeLike=variable&allele.fcode=functional&cloneName=&locusLike=IGH&mainLocusLike=IGH+locus&cosLocusLike=any&groupLike=any&subgroup=−1&geneLike=&selection=any”, the content of which is incorporated herein by reference. Thus, the VH chain may be selected from the group consisting of the amino acid sequences encoded by the genes IGHV1-18, IGHV1-2, IGHV1-24, IGHV1-3, IGHV1-45, IGHV1-46, IGHV1-58, IGHV1-69, IGHV1-69-2, IGHV1-69D, IGHV1-8, IGHV2-26, IGHV2-5, IGHV2-70, IGHV2-70D, IGHV3-11, IGHV3-13, IGHV3-15, IGHV3-20, IGHV3-21, IGHV3-23, IGHV3-23D, IGHV3-30, IGHV3-30-3, IGHV3-30-5, IGHV3-33, IGHV3-35, IGHV3-43, IGHV3-43D, IGHV3-48, IGHV3-49, IGHV3-53, IGHV3-62, IGHV3-64, IGHV3-64D, IGHV3-66, IGHV3-7, IGHV3-72, IGHV3-73, IGHV3-74, IGHV3-9, IGHV3-NL1, IGHV4-28, IGHV4-30-1, IGHV4-30-2, IGHV4-30-4, IGHV4-31, IGHV4-34, and IGHV4-38-2.

The human variable framework regions of the light chain (VK, kappa) may be retrieved from the website: “https://www.imgt.org/genedb/resultPage.action;jsessionid=49EB34C22C79EAC51862C76108963216?gene.id.species=Homo+sapiens&molComponent=IG&geneTypeLike=variable&allele.fcode=functional&cloneName=&locusLike=IGK&mainLocusLike=IGK+locus&cosLocusLike=any&groupLike=any&subgroup=−1 &geneLike=&selection=any”, the content of which is incorporated herein by reference. Thus, the VK (kappa) chain may be selected from the group consisting of the amino acid sequences encoded by the genes IGKV1-12, IGKV1-13, IGKV1-16, IGKV1-17, IGKV1-27, IGKV1-33, IGKV1-39, IGKV1-5, IGKV1-6, IGKV1-8, IGKV1-9, IGKV1-NL1, IGKV1D-12, IGKV1D-13, IGKV1D-16, IGKV1D-17, IGKV1D-33, IGKV1 D-39, IGKV1 D-43, IGKV1 D-8, IGKV2-24, IGKV2-28, IGKV2-29, IGKV2-30, IGKV2-40, IGKV2D-26, IGKV2D-28, IGKV2D-29, IGKV2D-30, IGKV2D-40, IGKV3-11, IGKV3-15, IGKV3-20, IGKV3D-11, IGKV3D-15, IGKV3D-20, IGKV3D-7, IGKV4-1, IGKV5-2, IGKV6-21, and IGKV6D-21.

The human variable framework regions of the light chain (VL, lambda) may be retrieved from the website: “https://www.imgt.org/genedb/resultPage.action;jsessionid=49EB34C22C79EAC51862C76108963216?gene.id.species=Homo+sapiens&molComponent=IG&geneTypeLike=variable&allele.fcode=functional&cloneName=&locusLike=IGL&mainLocusLike=IGL+locus&cosLocusLike=any&groupLike=any&subgroup=−1 &geneLike=&selection=any”, the content of which is incorporated herein by reference. Thus, the VL (lambda) chain may be selected from the group consisting of the amino acid sequences encoded by the genes IGLV1-36, IGLV1-40, IGLV1-44, IGLV1-47, IGLV1-51, IGLV10-54, IGLV2-11, IGLV2-14, IGLV2-18, IGLV2-23, IGLV2-8, IGLV3-1, IGLV3-10, IGLV3-12, IGLV3-16, IGLV3-19, IGLV3-21, IGLV3-22, IGLV3-25, IGLV3-27, IGLV3-9, IGLV4-3, IGLV4-60, IGLV4-69, IGLV5-37, IGLV5-39, IGLV5-45, IGLV5-52, IGLV6-57, IGLV7-43, IGLV7-46, IGLV8-61, and IGLV9-49.

The human variable framework region of the heavy and the light chain may also contain the respective human HJ (heavy chain), and light chain KJ (kappa) or LJ (lambda) sequences.

The HJ sequences may be retrieved from the website: “https://www.imgt.org/genedb/resultPage.action?gene.id.species=Homo+sapiens&molComponent=IG&geneTypeLike=any&allele.fcode=functional&cloneName=&locusLike=IGH&mainLocusLike=IGH+locus&cosLocusLike=any&groupLike=IGHJ&subgroup=−1 &geneLike=&selection=any”, the content of which is incorporated herein by reference. In particular, the HJ sequence may be selected from amino acid sequences encoded by the genes consisting of the group: IGHJ1, IGHJ2, IGHJ3, IGHJ4, IGHJ5, and IGHJ6.

The KJ sequences may be retrieved from the website: “https://www.imgt.org/genedb/resultPage.action?gene.id.species=Homo+sapiens&molComponent=IG&geneTypeLike=any&allele.fcode=functional&cloneName=&locusLike=IGK&mainLocusLike=IGK+locus&cosLocusLike=any&groupLike=IGKJ&subgroup=−1&geneLike=&selection=any”, the content of which is incorporated herein by reference. In particular, the KJ sequence may be selected from amino acid sequences encoded by the genes consisting of the group IGKJ1, IGKJ2, IGKJ3, IGKJ4, and IGKJ5.

The LJ sequences may be retrieved from the website: “https://www.imgt.org/genedb/resultPage.action?gene.id.species=Homo+sapiens&molComponent=IG&geneTypeLike=any&allele.fcode=functional&cloneName=&locusLike=IGL&mainLocusLike=IGL+locus&cosLocusLike=any&groupLike=IGLJ&subgroup=−1 &geneLike=&selection=any”, the content of which is incorporated herein by reference. In particular, the LJ sequence may be selected from amino acid sequences encoded by the genes consisting of the group: IGLJ1, IGLJ2, IGLJ3, IGLJ6, and IGLJ7.

The heavy chain CDR sequences of the invention (CDRH1, CDRH2 and CDRH3) may be comprised by a human heavy chain variable framework sequence as defined by a human VH sequence as described herein in combination with a human HJ sequence as described herein. Analogously, the light chain CDR sequences of the invention (CDRL1, CDRL2 and CDRL3) may be comprised by a human light chain variable framework sequence as defined by a human VL or VK sequence as described herein in combination with a human LJ or KJ sequence as described.

The combination of (i) a human VH sequence (comprising an CDRH1, an CDRH2 and an CDRH3 sequence, respectively, according to the invention) and of a human VL sequence (comprising an CDRL1, an CDRL2 and an CDRL3 sequence, respectively, of the invention) or of (ii) a human VH sequence (comprising an CDRH1, an CDRH2 and an CDRH3 sequence, respectively, of the invention) and of a human VK sequence (comprising an CDRL1, an CDRL2 and an CDRL3 sequence, respectively, of the invention) may thus characterize the variable framework region of an antibody according to the invention and its binding region. Also, the variable framework region may be characterized (i) by a human VH sequence and a human HJ sequence in combination with a human VL and a human LJ sequence or (ii) by a human VH sequence and a human HJ sequence in combination with a human VK and a human KJ sequence, thereby forming the variable framework region of an antibody according to the invention and its binding region.

The CDR sequences of the invention are inserted at the sites of the variable framework sequences which present the CDR sequences.

The numbering of the residues in the variable regions was done according to the IMGT numbering system (IMGT: http://www.imgt.org/; cf. Lefranc, M.-P. et al. (2009) Nucleic Acids Res. 37, D1006-D1012). To define the CDR regions, the Kabat CDR definition was applied (Tai Te Wu, Elvin A. Kabat; An analysis of the sequences of the variable regions of Bence Jones proteins and myeloma light chains and their implications for antibody complementarity. J Exp Med 1 Aug. 1970; 132 (2): 211-250; George Johnson, Tai Te Wu, Kabat Database and its applications: 30 years after the first variability plot, Nucleic Acids Research, Volume 28, Issue 1, 1 Jan. 2000, Pages 214-218).

TABLE 1
SEQ ID NOs for CDR sequences of antibodies
4E1, 25-3D3, 17B10, 6C9, 3F1, 13A2

| Antibody | CDRH1 | CDRH2 | CDRH3 | CDRL1 | CDRL2 | CDRL3 |
|---|---|---|---|---|---|---|
| 4E1 | 1 | 2 | 3 | 4 | 5 | 6 |
| 25-3D3 | 9 | 10 | 11 | 12 | 13 | 14 |
| 17B10 | 17 | 18 | 19 | 20 | 21 | 22 |
| 6C9 | 66 | 67 | 68 | 69 | 70 | 71 |
| 3F1 | 74 | 75 | 76 | 77 | 78 | 79 |
| 13A2 | 82 | 83 | 84 | 85 | 86 | 87 |

Furthermore, the amino acid sequences of the variable regions of the heavy chain (VH) and the light chain (VL) of exemplary antibodies 4E1, 25-3D3, 17B10, 6C9, 3F1, and 13A2 are shown in Table 2 below:

TABLE 2
SEQ ID NOs for VH/VL sequences of antibodies
4E1, 25-3D3, 17B10, 6C9, 3F1, and 13A2

| Antibody | VH | VL |
|---|---|---|
| 4E1 | 7 | 8 |
| 25-3D3 | 15 | 16 |
| 17B10 | 23 | 24 |
| 6C9 | 72 | 73 |
| 3F1 | 80 | 81 |
| 13A2 | 88 | 89 |

Preferably, the antibody of the invention, or the antigen-binding fragment thereof, comprises the combination of six CDR sequences of the exemplified antibodies shown in Table 1 (optionally the VH and VL sequences of the exemplified antibodies shown in Table 2), or sequence variants thereof, as defined herein.

In some embodiments, the antibody, or the antigen-binding fragment thereof, according to the present invention comprises a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6. Preferably, the antibody or the antigen-binding fragment thereof comprises:
a heavy chain CDR1 sequence according to SEQ ID NO: 1;
a heavy chain CDR2 sequence according to SEQ ID NO: 2;
a heavy chain CDR3 sequence according to SEQ ID NO: 3;
a light chain CDR1 sequence according to SEQ ID NO: 4;
a light chain CDR2 sequence according to SEQ ID NO: 5; and
a light chain CDR3 sequence according to SEQ ID NO: 6.

As shown in the appended examples, such an antibody (e.g., 4E1) binds specifically to Bet v 1, and also binds specifically or has affinity to several Bet v 1 homologous proteins, including tree pollen allergens, such as Cor a 1, Aln g 1, Fag s1, Que a 1, and Ost c 1, and food allergens, such as Mal d 1 and jug r 5.

In particular, as shown by epitope mapping experiments presented below, an antibody comprising the heavy chain and light chain CDR sequences according to SEQ ID Nos 1-6 (e.g. antibody 4E1) specifically binds to an epitope including the residues 46 (E), 54 (1), 56 (K), 69 (K) and 88 (E) of Bet v 1.

In some embodiments, such an antibody of the invention, or the antigen-binding fragment thereof, may comprise a heavy chain variable region VH comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 7, and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 8. Thereby, the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are preferably maintained.

In some embodiments, the antibody, or an antigen-binding fragment thereof, comprises a heavy chain variable region comprising or consisting of an amino acid sequence as set forth in SEQ ID NO: 7 and a light chain variable region comprising or consisting of an amino acid sequence as set forth in SEQ ID NO: 8.

In some embodiments, the antibody, or the antigen-binding fragment thereof, comprises a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14. Preferably, the antibody or the antigen-binding fragment thereof comprises:
a heavy chain CDR1 sequence according to SEQ ID NO: 9;
a heavy chain CDR2 sequence according to SEQ ID NO: 10;
a heavy chain CDR3 sequence according to SEQ ID NO: 11;
a light chain CDR1 sequence according to SEQ ID NO: 12;
a light chain CDR2 sequence according to SEQ ID NO: 13; and
a light chain CDR3 sequence according to SEQ ID NO: 14.

As shown in the appended examples, such an antibody (e.g., 25-3D3) binds specifically to Bet v 1, and also binds specifically or has affinity to several Bet v 1 homologous proteins, including e.g. Cor a 1, Aln g 1, Fag s1, Cas s1, Ost c 1, and Car b1, for example.

In particular, as shown by epitope mapping experiments presented below, an antibody comprising the heavy chain and light chain CDR sequences according to SEQ ID Nos 9-14 (e.g. antibody 25-3D3) specifically binds to an epitope including the residues 33 (K), 154 (A), 155 (H), and 157(D) of Bet v 1.

In some embodiments, such an antibody of the invention, or the antigen-binding fragment thereof, may comprise a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 15 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 16. Thereby, the CDR sequences as defined above (heavy chain CDR1, CDR2, and CDR3 sequences as set forth in SEQ ID NO: 9, SEQ ID NO: 10, and SEQ ID NO: 11, respectively; and light chain CDR1, CDR2, and CDR3 sequences as set forth in SEQ ID NO: 12, SEQ ID NO: 13, and SEQ ID NO: 14, respectively) are preferably maintained.

In some embodiments, the antibody, or an antigen-binding fragment thereof, comprises a heavy chain variable region comprising or consisting of an amino acid sequence as set forth in SEQ ID NO: 15 and a light chain variable region comprising or consisting of an amino acid sequence as set forth in SEQ ID NO: 16.

In some embodiments, the antibody, or the antigen-binding fragment thereof, comprises a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22. Preferably, the antibody or the antigen-binding fragment thereof comprises:
a heavy chain CDR1 sequence according to SEQ ID NO: 17;
a heavy chain CDR2 sequence according to SEQ ID NO: 18;
a heavy chain CDR3 sequence according to SEQ ID NO: 19;
a light chain CDR1 sequence according to SEQ ID NO: 20;
a light chain CDR2 sequence according to SEQ ID NO: 21; and
a light chain CDR3 sequence according to SEQ ID NO: 22.

As shown in the appended examples, such an antibody (e.g. 17B10) binds specifically to Bet v 1, and also binds specifically or has affinity to several Bet v 1 homologous proteins, including e.g. Cor a 1, Aln g 1, Cas s1, Ost c 1, and Car b1, for example.

In particular, as shown by epitope mapping experiments presented below, an antibody comprising the heavy chain and light chain CDR sequences according to SEQ ID Nos 17 to 22 (e.g. antibody 17B110) specifically binds to a Bet v 1 epitope including the residues 7 (E), 9 (E), and 131 (A) of Bet v 1.

In some embodiments, such an antibody of the invention, or the antigen-binding fragment thereof, may comprise a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 23 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 24. Thereby, the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21, and SEQ ID NO: 22, respectively) are preferably maintained.

In some embodiments, the antibody, or an antigen-binding fragment thereof, comprises a heavy chain variable region comprising or consisting of an amino acid sequence as set forth in SEQ ID NO: 23 and a light chain variable region comprising or consisting of an amino acid sequence as set forth in SEQ ID NO: 24.

In some embodiments, the antibody, or the antigen-binding fragment thereof, comprises a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at least 70% identity to SEQ ID NO: 70, and a CDRL3 having at least 70% identity to SEQ ID NO: 71.

Preferably, the antibody or the antigen-binding fragment thereof comprises:
a heavy chain CDR1 sequence according to SEQ ID NO: 66;
a heavy chain CDR2 sequence according to SEQ ID NO: 67;
a heavy chain CDR3 sequence according to SEQ ID NO: 68;
a light chain CDR1 sequence according to SEQ ID NO: 69;
a light chain CDR2 sequence according to SEQ ID NO: 70; and
a light chain CDR3 sequence according to SEQ ID NO: 71.

As shown in the appended examples, such an antibody (e.g. 6C9) binds specifically to Bet v 1, and also binds specifically or has affinity to several Bet v 1 homologous proteins, including e.g. Cor a 1, Aln g 1, Fag s 1, Que a 1, Cas s1, Carb b 1, Ost c 1, and Jur r 5, for example.

In particular, as shown by epitope mapping experiments presented below, an antibody comprising the heavy chain and light chain CDR sequences according to SEQ ID Nos 66-71 (e.g. antibody 6C9) specifically binds to an epitope including the residues 9 (E), 11 (T), 13 (V), 106 (V), 107 (A), 110 (D) and 116 (K) of Bet v 1.

In some embodiments, such an antibody of the invention, or the antigen-binding fragment thereof, may comprise a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 72 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 73. Thereby, the CDR sequences as defined above (heavy chain CDR1, CDR2, and CDR3 sequences as set forth in SEQ ID NO: 66, SEQ ID NO: 67, and SEQ ID NO: 68, respectively; and light chain CDR1, CDR2, and CDR3 sequences as set forth in SEQ ID NO: 69, SEQ ID NO: 70, and SEQ ID NO: 71, respectively) are preferably maintained.

In some embodiments, the antibody, or an antigen-binding fragment thereof, comprises a heavy chain variable region comprising or consisting of an amino acid sequence as set forth in SEQ ID NO: 72 and a light chain variable region comprising or consisting of an amino acid sequence as set forth in SEQ ID NO: 73.

In some embodiments, the antibody, or the antigen-binding fragment thereof, comprises a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79. Preferably, the antibody or the antigen-binding fragment thereof comprises:
a heavy chain CDR1 sequence according to SEQ ID NO: 74;
a heavy chain CDR2 sequence according to SEQ ID NO: 75;
a heavy chain CDR3 sequence according to SEQ ID NO: 76;
a light chain CDR1 sequence according to SEQ ID NO: 77;
a light chain CDR2 sequence according to SEQ ID NO: 78; and
a light chain CDR3 sequence according to SEQ ID NO: 79.

As shown in the appended examples, such an antibody (e.g. 3F1) binds specifically to Bet v 1, and also binds specifically or has affinity to several Bet v 1 homologous proteins, including e.g. Cor a 1, Fag s 1, Que a 1, Cas s1, and Jur r 5, for example.

As shown by epitope mapping experiments presented below, an antibody of the 6C9 clonotype specifically binds to an epitope including the residues 9 (E), 11 (T), 13 (V), 106 (V), 107 (A), 110 (D) and 116 (K) of Bet v 1. Since an antibody comprising the heavy chain and light chain CDR sequences according to SEQ ID Nos 74-79 (e.g. antibody 3F1), is of the same clonotype and also belongs to the same complementation group as 6C9 (as shown by cross-competition experiments below), it is assumed that an antibody comprising the heavy chain and light chain CDR sequences according to SEQ ID Nos 74-79 (e.g. antibody 3F1) also will bind to the same epitope, i.e. including the residues 9 (E), 11 (T), 13 (V), 106 (V), 107 (A), 110 (D) and 116 (K) of Bet v 1.

In some embodiments, such an antibody of the invention, or the antigen-binding fragment thereof, may comprise a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 80 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 81. Thereby, the CDR sequences as defined above (heavy chain CDR1, CDR2, and CDR3 sequences as set forth in SEQ ID NO: 74, SEQ ID NO: 75, and SEQ ID NO: 76, respectively; and light chain CDR1, CDR2, and CDR3 sequences as set forth in SEQ ID NO: 77, SEQ ID NO: 78, and SEQ ID NO: 79, respectively) are preferably maintained.

In some embodiments, the antibody, or an antigen-binding fragment thereof, comprises a heavy chain variable region comprising or consisting of an amino acid sequence as set forth in SEQ ID NO: 80 and a light chain variable region comprising or consisting of an amino acid sequence as set forth in SEQ ID NO: 81.

In some embodiments, the antibody, or the antigen-binding fragment thereof, comprises a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87. Preferably, the antibody or the antigen-binding fragment thereof comprises:
a heavy chain CDR1 sequence according to SEQ ID NO: 82;
a heavy chain CDR2 sequence according to SEQ ID NO: 83;
a heavy chain CDR3 sequence according to SEQ ID NO: 84;
a light chain CDR1 sequence according to SEQ ID NO: 85;
a light chain CDR2 sequence according to SEQ ID NO: 86; and
a light chain CDR3 sequence according to SEQ ID NO: 87.

As shown in the appended examples, such an antibody (e.g. 13A2) binds specifically to Bet v 1, and also binds specifically or has affinity to several Bet v 1 homologous proteins, including Cor a 1, Ain g 1, Fag s 1, Que a 1, Cas s1, Carb b 1, and Jur r 5, for example.

As shown by epitope mapping experiments presented below, an antibody of the 6C9 clonotype specifically binds to an epitope including the residues 9 (E), 11 (T), 13 (V), 106 (V), 107 (A), 110 (D) and 116 (K) of Bet v 1. As an antibody comprising the heavy chain and light chain CDR sequences according to SEQ ID Nos 82-87 (e.g. antibody 13A2), is of the same clonotype and also belongs to the same complementation group as 6C9 (as shown by cross-competition experiments below), it is assumed that an antibody comprising the heavy chain and light chain CDR sequences according to SEQ ID Nos 82-87 (e.g. antibody 13A2) will bind to the same epitope, i.e. including the residues 9 (E), 11 (T), 13 (V), 106 (V), 107 (A), 110 (D) and 116 (K) of Bet v 1.

In some embodiments, such an antibody of the invention, or the antigen-binding fragment thereof, may comprise a heavy chain variable region (VH) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 88 and a light chain variable region (VL) comprising an amino acid sequence having 70% or more (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%) identity to SEQ ID NO: 89. Thereby, the CDR sequences as defined above (heavy chain CDR1, CDR2, and CDR3 sequences as set forth in SEQ ID NO: 82, SEQ ID NO: 83, and SEQ ID NO: 84, respectively; and light chain CDR1, CDR2, and CDR3 sequences as set forth in SEQ ID NO: 85, SEQ ID NO: 86, and SEQ ID NO: 87, respectively) are preferably maintained.

In some embodiments, the antibody, or an antigen-binding fragment thereof, comprises a heavy chain variable region comprising or consisting of an amino acid sequence as set forth in SEQ ID NO: 88 and a light chain variable region comprising or consisting of an amino acid sequence as set forth in SEQ ID NO: 89.

In general, the antibody, or the antigen-binding fragment thereof, of the invention may be capable of reducing, inhibiting or neutralizing allergen-mediated biological activity. In particular, the antibodies may be capable of reducing or inhibiting the binding of an IgE antibody to a birch pollen allergen, in particular Bet v 1, and, optionally to a bet v 1 homologous allergen, such as another tree pollen allergen and/or a food allergen, as described herein. Thus, the antibodies or binding fragments thereof according to the invention may decrease or inhibit the activation of the mast cells or basophils and therefore decrease or prevent the release of mediators (e.g. histamine, lipid mediators, leukotriene). Thereby, the antibodies described herein may inhibit allergy symptoms that would usually occur in the patient after contact with the allergen (e.g. contact with the eyes, nose or mouth or food uptake). Accordingly, the antibodies described herein may be capable of reducing, inhibiting or neutralizing allergen-mediated biological activity.

In some embodiments, the CDRs or the variable regions of the antibody, or the antigen-binding fragment thereof, are human or are derived from human CDR or variable region sequences. The exemplary antibodies 4E1, 25-3D3, 17B10, 6C9, 3F1 and 13A2 (wild-type) are human antibodies, isolated from human patients. A “human-derived” CDR or VH/VL sequence includes engineered human antibody sequences, wherein mutations were introduced in the originally human CDR or VH/VL sequences. For example, a human-derived CDR may differ from the fully human (wild-type) CDR sequence in that it contains up to 5, i.e. 1, 2, 3, 4 or 5 mutations, preferably up to 4 mutations, more preferably up to 3 mutations. For example, a human-derived VH or VL sequence may differ from the fully human (wild-type) VH or VL sequence in that it contains up to 10, i.e. 1, 2, 3, 4, 5, 6, 7, 8, 9 or 10 mutations, preferably up to 7 mutations, more preferably up to 5 mutations (e.g., in the framework regions).

In some embodiments, the antibody, or the antigen-binding fragment thereof, is a human antibody. In some embodiments, the antibody, or the antigen-binding fragment thereof, is a monoclonal antibody. For example, the antibody, or the antigen-binding fragment thereof, may be a human monoclonal antibody.

Human antibodies are advantageous as compared to antibodies of non-human origin, because non-human antibodies, including chimeric and humanized antibodies, can trigger an adverse immune response, which can lead to nausea, diarrhoea and flu-like symptoms. In more severe cases, these side-effects can even be lethal. Non-human antibody segments often trigger immune responses in humans (anti-drug antibodies (ADA)), thereby not only eliciting undesired side effects, but also reducing the efficacy of the non-human antibody in humans. In contrast thereto, antibodies retrieved from humans have a higher safety profile, as the antibodies have proven tolerability in the human body, which is combined with the outstanding affinity maturation typical of the human immune system. As used herein, the term “human antibodies” not only includes antibodies originally found in humans, but also sequence variants thereof, wherein specific amino acid residues (but not entire antibody segments) are mutated. In contrast to non-human and humanized antibodies, which usually contain entire antibody segments (such as entire sets of CDR sequences) of non-human origin, sequence variants of human antibodies typically contain only selective/specific mutations within select antibody segments (e.g., within a CDR or framework region and/or within a constant region; e.g. to modify the antibodies' affinity, functionality, half-life, etc.).

For example, a human antibody according to the present invention may comprise only a limited number of mutations per CDR (e.g. no more than 6, preferably no more than 5, more preferably no more than 4, even more preferably no more than 3, still more preferably no more than 2 and particularly preferably only a single mutation per CDR), as compared to the sequences shown in Table 1. In case of more than two mutations they may not occur in a consecutive manner (to avoid creating a non-human sequence segment). The same applies to the framework regions (or the entire VH/VL sequences) as well to the constant regions. The latter may carry, for example, specific modifications known in the art to modify the antibody's (Fc-related) functionality, as described herein below.

Preferably, the antibody is an IgG or IgA antibody. IgG and igA usually compete with IgE for binding sites on the allergen and thereby prevent recognition of allergens by IgE bound to Fes receptors on the surface of mast cells and basophils. This may include direct competition by binding to the same epitope or competition through steric hindrance. Furthermore, IgG antibodies bound to the allergen can lead to cross-linking of Fcs and inhibitory FcγRIIB receptors, resulting in the decrease of effector cell activity. Thereby the IgG and IgA antibodies or binding fragments thereof according to the invention can be used for the effective prevention or treatment of allergies. In some embodiments, the variable regions or the CDRs of the antibody as defined herein are derived from a (human) IgE antibody and grafted in a scaffold of an IgG or IgA antibody. Preferably, the scaffold is of a human IgG or IgA. Accordingly, the variable regions, portions thereof or the CDRs may be human and grafted in an antibody framework, which is preferably of human origin, but a distinct antibody type, such as IgG or IgA instead of IgE. Typically, the human-derived portions of the variable regions that are grafted into the antibody framework comprise the CDRs. Among IgG, IgG1 and IgG4 are preferred.

Accordingly, the antibody according to the present invention, or an antigen binding fragment thereof, may comprise an Fc moiety. The Fc moiety may be derived from human origin, e.g. from human IgA or IgG, such as IgG1, IgG2, IgG3, and/or IgG4, e.g. human IgG4.

As used herein, the term “Fc moiety” refers to a sequence derived from the portion of an immunoglobulin heavy chain beginning in the hinge region just upstream of the papain cleavage site (e.g., residue 216 in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof. A complete Fc moiety comprises at least a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody.

In some embodiments, in the context of the present invention an Fc moiety comprises at least one of: a hinge (e.g., upper, middle, and/or lower hinge region) domain, a CH2 domain, a CH3 domain, or a variant, portion, or fragment thereof. An Fc moiety may comprise at least a hinge domain, a CH2 domain or a CH3 domain. The Fc moiety may be a complete Fc moiety. The Fc moiety may also comprise one or more amino acid insertions, deletions, or substitutions relative to a naturally-occurring Fc moiety. For example, at least one of a hinge domain, CH2 domain or CH3 domain (or portion thereof) may be deleted.

It will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. Such functions include Fc receptor (FcR) binding, antibody half-life modulation, ADCC function, protein A binding, protein G binding, and complement binding. The portions of naturally occurring Fc moieties, which are responsible and/or essential for such functions are well known by those skilled in the art. In some embodiments, the antibody according to the present invention comprises a (complete) Fc moiety/Fc region, wherein the interaction/binding with the Fc receptor is not compromised.

In general, binding of the antibody to an Fc receptor may be assessed by various methods known to the skilled person, such as ELISA (Hessell A J, Hangartner L, Hunter M, Havenith C E G, Beurskens F J, Bakker J M, Lanigan C M S, Landucci G, Forthal D N, Parren P W H I, et al.: Fc receptor but not complement binding is important in antibody protection against HIV. Nature 2007, 449:101-104; Grevys A, Bern M, Foss S, Bratlie D B, Moen A, Gunnarsen K S, Aase A, Michaelsen T E, Sandlie I, Andersen J T: Fc Engineering of Human IgG1 for Altered Binding to the Neonatal Fc Receptor Affects Fc Effector Functions. 2015, 194:5497-5508) or flow-cytometry (Perez L G, Costa M R, Todd C A, Haynes B F, Montefiori D C: Utilization of immunoglobulin G Fc receptors by human immunodeficiency virus type 1: a specific role for antibodies against the membrane-proximal external region of gp41. J Virol 2009, 83:7397-7410; Piccoli L, Campo I, Fregni C S, Rodriguez B M F, Minola A, Sallusto F, Luisetti M, Corti D, Lanzavecchia A: Neutralization and clearance of G M-CSF by autoantibodies in pulmonary alveolar proteinosis. Nat Commun 2015, 6:1-9).

In some embodiments, the antibody, or antigen binding fragment thereof, according to the present invention comprises an Fc region. As used herein, the term “Fc region” refers to the portion of an immunoglobulin formed by two or more Fc moieties of antibody heavy chains. For example, the Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2. The Fc region may be dimeric. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties.

In some embodiments, the Fc moiety, or the Fc region, comprises or consists of an amino acid sequence derived from a human immunoglobulin sequence (e.g., from an Fc region or Fc moiety from a human IgG molecule). However, the Fc moiety, or the Fc region, may comprise one or more amino acids from another mammalian species. For example, a primate Fc moiety or a primate binding site may be included in the antibody, or antigen-binding fragment. Alternatively, one or more murine amino acids may be present in the Fc moiety or in the Fc region.

The Fc moieties of the Fc region may be of the same or different class and/or subclass. For example, the Fc moieties may be derived from an immunoglobulin (e.g., a human immunoglobulin) of an IgG1, IgG2, IgG3 or IgG4 subclass.

Accordingly, antibodies of the invention can be of any isotype (e.g., IgA, IgG, IgM i.e. an α, γ or μ heavy chain). Preferably, the antibody may be of the IgA or IgG type. Within the IgG isotype, antibodies may be IgG1, IgG2, IgG3 or IgG4 subclass, preferably IgG1 or IgG4, more preferably IgG4. An exemplary sequence for an IgG4 constant region, which may be useful in the antibody as described herein, is provided in SEQ ID NO: 49. Accordingly, the antibody of the invention may comprise an amino acid sequence according to SEQ ID NO: 49, or a sequence variant thereof as described herein. The human IgG4 constant region sequence of SEQ ID NO: 49 comprises the stable hinge mutation S228P (S. Angal, D. J. King, M. W. Bodmer, A. Turner, A. D. G. Lawson, G. Roberts, B. Pedley, J. R. Adair, A single amino acid substitution abolishes the heterogeneity of chimeric mouse/human (IgG4) antibody, Molecular Immunology, Volume 30, Issue 1, 1993, Pages 105-108, ISSN 0161-5890, https://doi.org/10.1016/0161-5890(93)90432-B). Antibodies of the invention may have a κ or a λ light chain. Exemplary sequences for κ and λ light chain constant regions, which may be useful in the antibody as described herein, are provided in SEQ ID NO: 50 (kappa LC) and SEQ ID NO: 51 (lambda LC). Accordingly, the antibody of the invention may comprise an amino acid sequence according to SEQ ID NO: 50 or 51, or a sequence variant thereof as described herein.

As outlined above, the present invention encompasses antigen-binding fragments. An antigen-binding fragment may or may not comprise an Fc moiety, in particular a portion of a complete Fc region. In some embodiments, the antibody, or antigen-binding fragment thereof, is selected from Fab, Fab′, F(ab′)2, Fv or scFv. For example, F(ab′)2 (which may be obtained by pepsin cleavage or recombinant expression) as well as Fab′ (which can be obtained from F(ab′)2 or by recombinant expression) usually includes the hinge region.

In some embodiments, the antibody, or antigen-binding fragment, may be a single-chain antibody (or fragment). The single-chain antibody (or fragment) may encode the complete set of six CDRs, i.e. include the three heavy chain CDRs as well as the three light chain CDRs. More specifically, the single-chain antibody (or fragment) may include a heavy chain variable region (VH) as well as a light chain variable region (VL), for example including the VH and VL sequences as described above.

Variant antibodies are also included within the scope of the invention. Thus, variants of the sequences recited in the application are also included within the scope of the invention. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Antibodies of the invention, or antigen-binding fragments thereof, may be provided in purified form. Typically, the antibody, or antigen-binding fragment, will be present in a composition that is substantially free of other polypeptides e.g., where less than 90% (by weight), usually less than 60% and more usually less than 50% of the composition is made up of other polypeptides.

Antibodies of the invention may be immunogenic in non-human (or heterologous) hosts e.g., in mice. In particular, the antibodies may have an idiotope that is immunogenic in non-human hosts, but not in a human host. In particular, antibodies of the invention for human use include those that cannot be easily isolated from hosts such as mice, goats, rabbits, rats, non-primate mammals, etc. and cannot generally be obtained by humanization or from xeno-mice.

Antibodies of the invention also include hybrid antibody molecules that comprise the six CDRs from an antibody of the invention as defined above and one or more CDRs from another antibody to an antigen. For example, the antibody may be multispecific. In other embodiments, the antibody, or the antigen-binding fragment thereof, may be monospecific.

Accordingly, the antibody as described herein, or the antigen-binding fragment thereof, may be a multispecific antibody or a multispecific antigen-binding fragment.

As used herein, the term “multispecific” refers to the ability to bind to at least two different epitopes, e.g. on different antigens or on the same antigen. While a conventional monospecific IgG-type antibodies usually have two identical epitope binding sites (paratopes) and can, thus, only bind to identical epitopes (but not to different epitopes). A multispecific antibody, in contrast, has at least two different types of paratopes (antigen-binding sites) and can, thus, bind to at least two different epitopes. As used herein, “paratope” refers to an antigen-binding site (or epitope-binding site) of the antibody. Moreover, a single “specificity” may refer to one, two, three or more identical paratopes in a single antibody (the actual number of paratopes in one single antibody molecule is referred to as “valency”). For example, a single native IgG antibody is monospecific and bivalent, since it has two identical paratopes. Accordingly, a multispecific antibody comprises at least two (different) paratopes. Thus, the term “multispecific antibodies” refers to antibodies having more than one paratope and the ability to bind to two or more different epitopes. The term “multispecific antibodies” comprises in particular bispecific antibodies, but typically also protein, e.g. antibody scaffolds, which bind in particular to three or more different epitopes, i.e. antibodies with three or more different paratopes.

In particular, the multispecific antibody, or the multispecific antigen binding fragment, of the invention may comprise two or more paratopes, wherein one or more paratopes may be identical so that all paratopes of the antibody belong to at least two different types of paratopes and, hence, the antibody has at least two specificities. For example, the multispecific antibody or antigen binding fragment thereof according to the present invention may comprise four paratopes, wherein each two paratopes are identical (i.e. have the same specificity) and, thus, the antibody or fragment thereof is bispecific and tetravalent (two identical paratopes for each of the two specificities). Thus, “one specificity” refers in particular to one or more paratopes exhibiting the same specificity (which typically means that such one or more paratopes are identical) and, thus, “two specificities” may be realized by two, three, four five, six or more paratopes as long as they refer to only two specificities. In some embodiments, the multispecific antibody comprises one single paratope for each (of the at least two) specificity, i.e. the multispecific antibody comprises in total at least two paratopes. For example, a bispecific antibody may comprise one single paratope for each of the two specificities, i.e. the antibody comprises in total two paratopes. In other embodiments, the antibody comprises two (identical) paratopes for one or more of the specificities.

Preferably, the multispecific antibody or the multispecific antigen-binding fragment is bispecific or trispecific. As used herein, terms like “bispecific”, trispecific”, etc. refer to the number of different epitopes to which the antibody can bind to. For example, a “bispecific” antibody has exactly two different specificities (two different antigen-binding sites, wherein each of the two different antigen-binding sites may independently occur once or more than once, e.g. twice). For example, a “trispecific” antibody has exactly three different specificities (three different antigen-binding sites, wherein each of the three different antigen-binding sites may independently occur once or more than once, e.g. twice).

Various such multispecific antibody formats and methods for obtaining multispecific antibodies are known in the art. For example, building blocks for bispecific and trispecific antibodies are described in Xiufeng Wu, Stephen J. Demarest, Building blocks for bispecific and trispecific antibodies, Methods, Volume 154, 2019, Pages 3-9, ISSN 1046-2023, https://doi.org/10.1016/j.ymeth.2018.08.010, which is incorporated herein by reference. Methods for obtaining multispecific antibodies and further multispecific antibody formats are described in Amaral M, Holper S, Lange C, Jung J, Sjuts H, Weil S, Fischer M, Radoevic K, Rao E. Engineered Technologies and Bioanalysis of multispecific antibody formats. J Appl Bioanal 6(1), 26-51 (2020), which is incorporated herein by reference.

In some embodiments, the multispecific antibody, or the multispecific antigen binding fragment, according to the present invention is a bispecific antibody or a bispecific antigen binding fragment. Bispecific antibodies comprise (exactly) two specificities. A bispecific antibody in the context of the present invention may be of any bispecifc antibody format known in the art, e.g., as described in Spiess C., Zhai Q. and Carter P. J. (2015) Molecular Immunology 67: 95-106. For example, bispecific antibodies may be whole antibodies, such as whole IgG-like molecules, or fragments thereof which are not whole antibodies but retain antibody properties. These may be small recombinant formats, e.g. as tandem single chain variable fragment molecules (taFvs), diabodies (Dbs), single chain diabodies (scDbs), and various other derivatives of these (e.g., as described in Byrne H. et al. (2013) Trends Biotech, 31 (11): 621-632 with FIG. 2 showing various bispecific antibody formats). In some embodiments, the bispecific antibody may be an IgG(H)-scFv fusion as described in Coloma, M., Morrison, S. Design and production of novel tetravalent bispecific antibodies. Nat Biotechnol 15, 159-163 (1997). https://doi.org/10.1038/nbt0297-159.

In some embodiments, the multispecific antibody, or the multispecific antigen binding fragment, according to the present invention is a trispecific antibody or a trispecific antigen binding fragment. Trispecific antibodies comprise (exactly) three specificities. A trispecific antibody in the context of the present invention may be of any trispecifc antibody format known in the art. In some embodiments, the trispecific antibody may be a heterodimer of IgG(H)-scFvs with same Fab domains using knob-into-hole CH3 as described in Ridgway J B, Presta L G, Carter P. ‘Knobs-into-holes’ engineering of antibody CH3 domains for heavy chain heterodimerization. Protein Eng. 1996 July; 9(7):617-21. doi: 10.1093/protein/9.7.617.

Usually, the multispecific antibody, or the multispecific antigen binding fragment, is at least bivalent, i.e. it has at least two paratopes. Preferably, the multispecific antibody, or the multispecific antigen binding fragment, is bivalent, trivalent, tetravalent, or hexavalent. More preferably, the multispecific antibody, or the multispecific antigen binding fragment, is tetravalent. Even more preferably, the multispecific antibody, or the multispecific antigen binding fragment, is tetravalent and bispecific, or trispecific.

Preferably, the multispecific antibody, or the multispecific antigen binding fragment, binds (specifically) to distinct, non-overlapping epitopes of Bet v 1, and optionally binds (specifically) to distinct, non-overlapping epitopes of at least one Bet v 1 homologous protein. Accordingly, the antigen-binding sites (paratopes) of the multispecific antibody, or the multispecific antigen binding fragment, preferably target different, non-overlapping epitopes on the same antigen, namely Bet v1, and optionally a Bet v 1 homologous protein.

To this end, the antigen-binding sites (paratopes) of the multispecific antibody, or the multispecific antigen binding fragment, may be derived from the antibodies 4E1, 25-3D3, 17B10, 6C9, 3F1 and 13A2, respectively, as disclosed herein, which preferably bind to distinct, non-overlapping epitopes of Bet v 1. The skilled artisan is well aware of methods for obtaining a multispecific antibody using the binding sites of monospecific antibodies, e.g. as described above.

Accordingly, the multispecific antibody, or the multispecific antigen binding fragment, may comprise at least two of the following:
(i) an antigen-binding site comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6; in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antigen-binding site comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14; in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
(iii) an antigen-binding site comprising a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22; in particular a CDRH1 according to SEQ ID NO: 17, a CDRH2 according to SEQ ID NO: 18, a CDRH3 according to SEQ ID NO: 19, a CDRL1 according to SEQ ID NO: 20, a CDRL2 according to SEQ ID NO: 21, and a CDRL3 according to SEQ ID NO: 22;
(iv) an antigen-binding site comprising a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at least 70% identity to SEQ ID NO: 70, and a CDRL3 having at least 70% identity to SEQ ID NO: 71; in particular a CDRH1 according to SEQ ID NO: 66, a CDRH2 according to SEQ ID NO: 67, a CDRH3 according to SEQ ID NO: 68, a CDRL1 according to SEQ ID NO: 69, a CDRL2 according to SEQ ID NO: 70, and a CDRL3 according to SEQ ID NO: 71;
(v) an antigen-binding site comprising a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79, in particular a CDRH1 according to SEQ ID NO: 74, a CDRH2 according to SEQ ID NO: 75, a CDRH3 according to SEQ ID NO: 76, a CDRL1 according to SEQ ID NO: 77, a CDRL2 according to SEQ ID NO: 78, and a CDRL3 according to SEQ ID NO: 79;
(vi) an antigen-binding site comprising a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87, in particular a CDRH1 according to SEQ ID NO: 82, a CDRH2 according to SEQ ID NO: 83, a CDRH3 according to SEQ ID NO: 84, a CDRL1 according to SEQ ID NO: 85, a CDRL2 according to SEQ ID NO: 86, and a CDRL3 according to SEQ ID NO: 87.

In preferred embodiments, the multispecific antibody, or the multispecific antigen binding fragment, is a trispecific antibody or trispecific antigen binding fragment comprising three antigen-binding sites selected from (i) to (vi) as defined above, and preferably comprises a first antigen-binding site according to (i), an second antigen-binding site according to (ii), and a third antigen-binding site selected from any one of (iii) to (vi).

In some embodiments, the multispecific antibody, or the multispecific antigen binding fragment, comprises at least two of the following:
(i) an antigen-binding site comprising a VH having at least 70% identity to SEQ ID NO: 7, and a VL having at least 70% identity to SEQ ID NO: 8, wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained, in particular, a VH according to SEQ ID NO: 7, and a VL according to SEQ ID NO: 8;
(ii) an antigen-binding site comprising a VH having at least 70% identity to SEQ ID NO: 15, and a VL having at least 70% identity to SEQ ID NO: 16, wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained, in particular, a VH according to SEQ ID NO: 15, and a VL according to SEQ ID NO: 16;
(iii) an antigen-binding site comprising a VH having at least 70% identity to SEQ ID NO: 23, and a VL having at least 70% identity to SEQ ID NO: 24, wherein the CDRH1 according to SEQ ID NO: 17, CDRH2 according to SEQ ID NO: 18, CDRH3 according to SEQ ID NO: 19, CDRL1 according to SEQ ID NO: 20, CDRL2 according to SEQ ID NO: 21, and CDRL3 according to SEQ ID NO: 22 are preferably maintained, in particular, a VH according to SEQ ID NO: 23, and a VL according to SEQ ID NO: 24;
(iv) an antigen-binding site comprising a VH having at least 70% identity to SEQ ID NO: 72, and a VL having at least 70% identity to SEQ ID NO: 73, wherein the CDRH1 according to SEQ ID NO: 66, CDRH2 according to SEQ ID NO: 67, CDRH3 according to SEQ ID NO: 68, CDRL1 according to SEQ ID NO: 69, CDRL2 according to SEQ ID NO: 70, and CDRL3 according to SEQ ID NO: 71 are preferably maintained, in particular, a VH according to SEQ ID NO: 72, and a VL according to SEQ ID NO: 73;
(v) an antigen-binding site comprising a VH having at least 70% identity to SEQ ID NO: 80, and a VL having at least 70% identity to SEQ ID NO: 81, wherein the CDRH1 according to SEQ ID NO: 74, CDRH2 according to SEQ ID NO: 75, CDRH3 according to SEQ ID NO: 76, CDRL1 according to SEQ ID NO: 77, CDRL2 according to SEQ ID NO: 78, and CDRL3 according to SEQ ID NO: 79 are preferably maintained, in particular, a VH according to SEQ ID NO: 80, and a VL according to SEQ ID NO: 81;
(vi) an antigen-binding site comprising a VH having at least 70% identity to SEQ ID NO: 88, and a VL having at least 70% identity to SEQ ID NO: 89, wherein the CDRH1 according to SEQ ID NO: 82, CDRH2 according to SEQ ID NO: 83, CDRH3 according to SEQ ID NO: 84, CDRL1 according to SEQ ID NO: 85, CDRL2 according to SEQ ID NO: 86, and CDRL3 according to SEQ ID NO: 87 are preferably maintained, in particular, a VH according to SEQ ID NO: 88, and a VL according to SEQ ID NO: 89.

In preferred embodiments, the multispecific antibody, or the multispecific antigen binding fragment, is a trispecific antibody, or trispecific antigen binding fragment, comprising three antigen-binding sites selected from (i) to (vi) as defined above, and preferably comprises a first antigen-binding site according to (i), an second antigen-binding site according to (ii), and a third antigen-binding site selected from any one of (iii) to (vi).

In a preferred embodiment, the multispecific antibody, or the multispecific antigen binding fragment, comprises at least two of the following:
(i) an antigen-binding site comprising (a) the CDRH1, CDRH2, CDRH3, CDRL1, CDRL2 and CDRL3 sequences and/or (b) the VH and VL sequences of 4E1, or sequence variants thereof, as described above;
(ii) an antigen-binding site comprising (a) the CDRH1, CDRH2, CDRH3, CDRL1, CDRL2 and CDRL3 sequences and/or (b) the VH and VL sequences of 25-3D3, or sequence variants thereof, as described above;
(iii) an antigen-binding site comprising (a) the CDRH1, CDRH2, CDRH3, CDRL1, CDRL2 and CDRL3 sequences and/or (b) the VH and VL sequences of 17B110, or sequence variants thereof, as described above;
(iv) an antigen-binding site comprising (a) the CDRH1, CDRH2, CDRH3, CDRL1, CDRL2 and CDRL3 sequences and/or (b) the VH and VL sequences of 6C), or sequence variants thereof, as described above;
(v) an antigen-binding site comprising (a) the CDRH1, CDRH2, CDRH3, CDRL1, CDRL2 and CDRL3 sequences and/or (b) the VH and VL sequences of 3F1, or sequence variants thereof, as described above;
(vi) an antigen-binding site comprising (a) the CDRH1, CDRH2, CDRH3, CDRL1, CDRL2 and CDRL3 sequences and/or (b) the VH and VL sequences of 13A2, or sequence variants thereof, as described above.

in some embodiments, the multispecific antibody, or the multispecific antigen binding fragment, may be a bispecific antibody or a bispecific antigen binding fragment comprising:
(i) an antigen-binding site comprising (a) the CDRH1, CDRH2, CDRH3, CDRL1, CDRL2 and CDRL3 sequences and/or (b) the VH and VL sequences of 4E1, or sequence variants thereof, as described above; and
(ii) an antigen-binding site comprising (a) the CDRH1, CDRH2, CDRH3, CDRL1, CDRL2 and CDRL3 sequences and/or (b) the VH and VL sequences of 25-3D3, or sequence variants thereof, as described above.

In some embodiments, the multispecific antibody, or the multispecific antigen binding fragment, may be a bispecific antibody or a bispecific antigen binding fragment comprising:
(i) an antigen-binding site comprising (a) the CDRH1, CDRH2, CDRH3, CDRL1, CDRL2 and CDRL3 sequences and/or (b) the VH and VL sequences of 4E1, or sequence variants thereof, as described above; and
(iii) an antigen-binding site comprising (a) the CDRH1, CDRH2, CDRH3, CDRL1, CDRL2 and CDRL3 sequences and/or (b) the VH and VL sequences of 17B110, or sequence variants thereof, as described above.

In some embodiments, the multispecific antibody, or the multispecific antigen binding fragment, may be a bispecific antibody or a bispecific antigen binding fragment comprising:
(ii) an antigen-binding site comprising (a) the CDRH1, CDRH2, CDRH3, CDRL1, CDRL2 and CDRL3 sequences and/or (b) the VH and VL sequences of 25-3D3, or sequence variants thereof, as described above; and
(iii) an antigen-binding site comprising (a) the CDRH1, CDRH2, CDRH3, CDRL1, CDRL2 and CDRL3 sequences and/or (b) the VH and VL sequences of 17B110, or sequence variants thereof, as described above.

In preferred embodiments, the multispecific antibody, or the multispecific antigen binding fragment, may be a trispecific antibody or a trispecific antigen binding fragment comprising:
(i) a first antigen-binding site comprising (a) the CDRH1, CDRH2, CDRH3, CDRL1, CDRL2 and CDRL3 sequences and/or (b) the VH and VL sequences of 4E1, or sequence variants thereof, as described above;
(ii) a second antigen-binding site comprising (a) the CDRH1, CDRH2, CDRH3, CDRL1, CDRL2 and CDRL3 sequences and/or (b) the VH and VL sequences of 25-3D3, or sequence variants thereof, as described above; and a third antigen-binding site selected from the group consisting of (iii) to (vi):
(iii) an antigen-binding site comprising (a) the CDRH1, CDRH2, CDRH3, CDRL1, CDRL2 and CDRL3 sequences and/or (b) the VH and VL sequences of 17B110, or sequence variants thereof, as described above;
(iv) an antigen-binding site comprising (a) the CDRH1, CDRH2, CDRH3, CDRL1, CDRL2 and CDRL3 sequences and/or (b) the VH and VL sequences of 6C9, or sequence variants thereof, as described above;
(v) an antigen-binding site comprising (a) the CDRH1, CDRH2, CDRH3, CDRL1, CDRL2 and CDRL3 sequences and/or (b) the VH and VL sequences of 3F1, or sequence variants thereof, as described above; and
(vi) an antigen-binding site comprising (a) the CDRH1, CDRH2, CDRH3, CDRL1, CDRL2 and CDRL3 sequences and/or (b) the VH and VL sequences of 13A2, or sequence variants thereof, as described above.

In other words, in a specific embodiment, the antigen-binding sites (paratopes) of the multispecific antibody, or the multispecific antigen binding fragment, may be selected from the three different antibodies 4E1, 25-3D3 and 17B110, or the variants thereof, as described above, in particular as shown in Tables 1 and 2. In another specific embodiment, the antigen-binding sites (paratopes) of the multispecific antibody, or the multispecific antigen binding fragment, may be selected from the three different antibodies 4E1, 25-3D3 and 6C9. In a further specific embodiment, the antigen-binding sites (paratopes) of the multispecific antibody, or the multispecific antigen binding fragment, may be selected from the three different antibodies 4E1, 25-3D3 and 3F1. In a still further specific embodiment, specific embodiment, the antigen-binding sites (paratopes) of the multispecific antibody, or the multispecific antigen binding fragment, may be selected from the three different antibodies 4E1, 25-3D3 and 13A2.

In some embodiments, the multispecific antibody, or the multispecific antigen binding fragment, is bispecific and comprises two distinct antigen-binding sites selected from (i) to (vi). Such a bispecific antibody may be combined, in use, with a second (monospecific) antibody comprising the remaining antigen-binding site selected from (i) to (vi) (i.e. with the antigen-binding site selected from (i) to (vi), which is not contained in the (first) bispecific antibody). For example, a bispecific antibody, containing two distinct antigen-binding sites selected from (i) to (iii) may be combined with a second antibody, such that the combination of the bispecific antibody with the monospecific antibody includes all three antigen-binding sites of (i) to (iii). For combination, the antibodies may be comprised in the same composition or in different compositions (but administered in a combined treatment schedule).

### Nucleic Acids

In another aspect, the invention also provides a nucleic acid molecule comprising a polynucleotide encoding the antibody according to the present invention, or an antigen-binding fragment thereof, as described above.

In some embodiments, the nucleic acid molecule comprises one or more polynucleotide(s) encoding the exemplified antibodies of the invention (e.g., as described above, in particular in Tables 1 and 2), or a sequence variant thereof (e.g., having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity as described above).

Exemplified nucleic acid sequences encoding the CDR and VH/VL sequences of exemplified antibodies as described herein are shown in Table 3 below.

TABLE 3
SEQ ID NOs for polynucleotide sequences encoding the CDR and VH/VL sequences
of fully human antibodies 4E1, 25-3D3, 17B10, 6C9, 3F1 and 13A2.

| Antibody | CDRH1 | CDRH2 | CDRH3 | CDRL1 | CDRL2 | CDRL3 | VH | VL |
|---|---|---|---|---|---|---|---|---|
| 4E1 | 25 | 26 | 27 | 28 | 29 | 30 | 31 | 32 |
| 25-3D3 | 33 | 34 | 35 | 36 | 37 | 38 | 39 | 40 |
| 17B10 | 41 | 42 | 43 | 44 | 45 | 46 | 47 | 48 |
| 6C9 | 90 | 91 | 92 | 93 | 94 | 95 | 96 | 97 |
| 3F1 | 98 | 99 | 100 | 101 | 102 | 103 | 104 | 105 |
| 13A2 | 106 | 107 | 108 | 109 | 110 | 111 | 112 | 113 |

Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA. Nucleic acids may encode the light chain and/or the heavy chain of an antibody (or a single chain antibody). In other words, the light chain and the heavy chain of the antibody may be encoded by the same nucleic acid molecule (e.g., for single chain antibodies or for antibodies with separate heavy and light chains in bicistronic manner or an expression cassette containing more than one ribosome entry site such as IRES). Alternatively, the light chain and the heavy chain of the antibody may be encoded by distinct nucleic acid molecules. In a similar manner, for multispecific antibodies comprising two or more immunoglobulin chains, the different chains may be encoded by the same nucleic acid molecule (e.g., in a multicistronic manner or an expression cassette containing more than one ribosome entry site such as IRES). Alternatively, the different immunoglobulin chains of the multispecific antibody may be encoded by distinct nucleic acid molecules.

Due to the redundancy of the genetic code, the present invention also comprises sequence variants of nucleic acid sequences, which encode the same amino acid sequences. The polynucleotide encoding the antibody (or the complete nucleic acid molecule) may be optimized for expression of the antibody. For example, codon optimization of the nucleotide sequence may be used to improve the efficiency of translation in expression systems for the production of the antibody. Moreover, the nucleic acid molecule may comprise heterologous elements (i.e., elements, which in nature do not occur on the same nucleic acid molecule as the coding sequence for the (heavy or light chain of) an antibody. For example, a nucleic acid molecule may comprise a heterologous promotor, a heterologous enhancer, heterologous UTR (e.g., for optimal translation/expression), a heterologous poly-A-tail, heterologous DNA insulator elements and the like.

A nucleic acid molecule is a molecule comprising nucleic acid components. The term nucleic acid molecule usually refers to DNA or RNA molecules. It may be used synonymous with the term “polynucleotide”, i.e. the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent”, i.e. not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the antibody, or an antigen-binding fragment thereof, (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, J Cat: a novel tool to adapt codon usage of a target gene to its potential expression host. Nucleic Acids Res. 2005 Jul. 1; 33(Web Server issue):W526-31; or, for example, Genscript's OptimumGene™ algorithm (as described in US 2011/0081708 A1).

For example, the nucleic acid molecule of the invention may comprise a nucleic acid sequence as set forth in any one of SEQ ID NOs 25 to 48, and SEQ ID NOs 90 to 113; or a sequence variant thereof having at least 50%, at least 55%, at least 60%, at least 65%, at least 70%, at least 75%, at least 80%, at least 85%, at least 88%, at least 90%, at least 92%, at least 95%, at least 96%, at least 97%, at least 98% or at least 99% sequence identity. Thereby, the nucleic acid molecule may encode any one of the exemplified antibodies 4E1, 25-3D3, 17B110, 6C9, 3F1 and 13A2 (by combining the sequences as shown in Table 3), or a sequence variant thereof as described herein.

The present invention also provides a plurality of nucleic acid molecules encoding the antibody, or an antigen-binding fragment thereof, as described herein, wherein each of the nucleic acid molecules (of the plurality of nucleic acid molecules) comprises a polynucleotide encoding an immunoglobulin chain of the antibody, or an antigen-binding fragment thereof. Thereby, the plurality of nucleic acid molecules, taken together, encodes (all of the immunoglobulin chains of) the antibody, or an antigen-binding fragment thereof, as described herein. In some embodiments, the plurality of nucleic acid molecules encoding the antibody, or an antigen-binding fragment thereof, as described herein, may be a combination of a first and a second nucleic acid molecule, wherein the first nucleic acid molecule comprises a polynucleotide encoding the heavy chain of the antibody, or an antigen-binding fragment thereof, of the present invention; and the second nucleic acid molecule comprises a polynucleotide encoding the corresponding light chain of the same antibody, or the same antigen-binding fragment thereof.

In general, the above description regarding the (general) features of the nucleic acid molecule of the invention applies accordingly to the nucleic acid molecules of the plurality of nucleic acid molecules. Accordingly, one or more of the polynucleotides encoding the immunoglobulin chains of the antibody, or an antigen-binding fragment thereof, may be codon-optimized. For example, the plurality may comprise a nucleic acid sequence as set forth in any one of SEQ ID NOs 25 to 48, and SEQ ID NOs 90 to 113; or a sequence variant thereof having at least 50%, at least 55%, at least 60%, at least 65%, at least 70%, at least 75%, at least 80%, at least 85%, at least 88%, at least 90%, at least 92%, at least 95%, at least 96%, at least 97%, at least 98% or at least 99% sequence identity. Thereby, the plurality of nucleic acid molecules may encode any one of the exemplified antibodies 4E1, 25-3D3, 17B110, 6C9, 3F1 and 13A2 (by combining the sequences as shown in Table 3), or a sequence variant thereof as described herein.

### Vectors

Further included within the scope of the invention are vectors, for example, expression vectors, comprising a nucleic acid molecule according to the present invention or the plurality of nucleic acid molecules according to the present invention. Usually, a vector comprises a nucleic acid molecule as described above.

The present invention also provides a plurality of vectors comprising the plurality of nucleic acid molecules according to invention as described above. Thereby, each vector of the plurality of vectors may contain one or more nucleic acid molecules of the plurality of nucleic acid molecules according to invention as described above. In some embodiments, the plurality of vectors may be a combination of a first and a second vector, wherein the first vector comprises a first nucleic acid molecule as described above (for the combination of nucleic acid molecules) and the second vector comprises a second nucleic acid molecule as described above (for the combination of nucleic acid molecules).

A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multiple cloning site, a heterologous promotor, a heterologous enhancer, a heterologous selection marker (to identify cells comprising said vector in comparison to cells not comprising said vector), heterologous origin of replications, heterologous DNA insulator elements and the like. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a (heavy and/or light chain of a) desired antibody according to the present invention. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

As used herein, the term “vector” may also refer to a delivery vector, e.g. for viral or non-viral delivery of a nucleic acid of the invention. Alternatively, it may be referred to viral or non-viral delivery systems. Accordingly, the present invention also provides a delivery vector/system comprising the nucleic acid molecule as described above (or comprising an expression vector as described above). The delivery vector/system may be viral or non-viral.

Various examples of viral and non-viral delivery vectors/systems are known in the art and described, for example, in Nayerossadat N, Maedeh T, Ali PA. Viral and nonviral delivery systems for gene delivery. Adv Biomed Res. 2012; 1:27. doi:10.4103/2277-9175.98152, which is incorporated herein by reference. Non-limiting examples of viral delivery vectors/systems include retroviral vectors; adenoviral vectors; adeno-associated viral (AAV) vectors, including helper-dependent adenoviral vectors and hybrid adenoviral vectors; herpes simplex virus vectors; lentivirus vectors; poxvirus vectors and Epstein-Barr virus vectors. Among the viral vectors, adenoviral vectors and adeno-associated viral (AAV) vectors are preferred. Non-limiting examples of non-viral delivery vectors/systems include chemical and non-chemical methods. Non-chemical delivery includes physical methods, such as electroporation and other methods for transient penetration of the cell membrane by mechanical, electrical, ultrasonic, hydrodynamic, or laser-based energy; naked DNA or RNA delivery; gene gun; hydrodynamic delivery; ultrasound delivery and magnetofection. Chemical non-viral delivery systems include cationic particles, in particular cationic lipids/liposomes, cationic polymers and lipid/polymer systems. Among non-viral vectors/systems, cationic liposomes are preferred.

### Cells

In a further aspect, the present invention also provides a (host) cell expressing the antibody according to the present invention, or an antigen-binding fragment thereof; and/or comprising the vector (or the plurality of vectors) according the present invention. The (host) cell may be an isolated cell, which is not part of a human or animal body, e.g. a cell line or an engineered cell. The cell may express the nucleic acid(s) or vector(s) of the invention in a recombinant manner, e.g. in a heterologous manner (i.e., the cell/cell type does not express the antibody or the antigen-binding fragment in nature).

Examples of such cells include, but are not limited to, eukaryotic cells, e.g., yeast cells, animal cells or plant cells. Other examples of such cells include, but are not limited to, prokaryotic cells, e.g. E co/i. In some embodiments, the cells are mammalian cells, such as a mammalian cell line. Examples include human cells, CHO cells, HEK293 cells, PER.C6 cells, NS0 cells, human liver cells, myeloma cells or hybridoma cells.

The cell may be transfected with a vector according to the present invention, for example with an expression vector. The term “transfection” refers to the introduction of nucleic acid molecules, such as DNA or RNA (e.g. mRNA) molecules, into cells, e.g. into eukaryotic or prokaryotic cells. In the context of the present invention, the term “transfection” encompasses any method known to the skilled person for introducing nucleic acid molecules into cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

Moreover, the cells of the present invention may be transfected stably or transiently with the vector according to the present invention, e.g. for expressing the antibody according to the present invention. In some embodiments, the cells are stably transfected with the vector according to the present invention encoding the antibody according to the present invention. In other embodiments, the cells are transiently transfected with the vector according to the present invention encoding the antibody according to the present invention.

Accordingly, the present invention also provides a recombinant host cell, which heterologously expresses the antibody of the invention or the antigen-binding fragment thereof. For example, the cell may be of another species than the antibody (e.g., CHO cells expressing human antibodies). In some embodiments, the cell type of the cell does not express (such) antibodies in nature. Moreover, the host cell may impart a post-translational modification (PTM; e.g., glycosylation) on the antibody that is not present in their native state. Such a PTM may result in a functional difference (e.g., decreased immunogenicity). Accordingly, the antibody of the invention, or the antigen-binding fragment thereof, may have a post-translational modification, which is distinct from the naturally produced antibody (e.g., an antibody of an immune response in a human).

### Production of Antibodies

Antibodies according to the invention can be made by any method known in the art. For example, the general methodology for making monoclonal antibodies using hybridoma technology is well known (Kohler, G. and Milstein, C., 1975; Kozbar et al. 1983).

Standard techniques of molecular biology may be used to prepare DNA sequences encoding the antibodies or antigen-binding fragments of the present invention. Desired DNA sequences may be synthesized completely or in part, e.g., using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention. Eukaryotic, e.g., mammalian, host cell expression systems may be used for production of antibody molecules, such as complete antibody molecules. Suitable mammalian host cells include, but are not limited to, CHO, HEK293, PER.C6, NS0, myeloma or hybridoma cells. Also, prokaryotic, e.g. bacterial host cell expression systems may be used for the production of antibody molecules, such as complete antibody molecules. Suitable bacterial host cells include, but are not limited to, E. coli cells.

Accordingly, the present invention provides a method for preparing the antibody, or an antigen-binding fragment or an immunoglobulin chain(s) thereof, according to the present invention, said method comprising
(i) culturing the host cell as described above; and
(ii) isolating the antibody or immunoglobulin chain(s) thereof from the culture.

In other words, the present invention also provides a process for the production of an antibody molecule according to the present invention comprising culturing a (heterologous) host cell comprising a vector encoding a nucleic acid of the present invention, in particular under conditions suitable for expression of protein from DNA encoding the antibody molecule of the present invention, and isolating the antibody molecule.

For production of the antibody comprising both heavy and light chains, a host cell, such as a cell line, may be transfected with two vectors, a first vector encoding a light chain polypeptide and a second vector encoding a heavy chain polypeptide, e.g. as described above. Alternatively, a single vector may be used, the vector including sequences encoding light chain and heavy chain polypeptides (e.g. for single chain antibodies or in a bicistronic manner). Likewise, a plurality of vectors may be used, if multispecific antibodies with two or more immunoglobulin chains shall be expressed.

Thus, the invention also provides a method for preparing a recombinant cell, comprising the steps of: (i) providing one or more nucleic acids that encode(s) the antibody of the invention; (ii) inserting the nucleic acid into an expression vector and (iii) transfecting the vector into a (heterologous) host cell in order to permit expression of the antibody of interest in that host cell. The nucleic acid of step (i) may, but need not, be manipulated to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest. Thus the procedures for first preparing the nucleic acid(s) and then using it to transfect a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

The transfected host cell may be a eukaryotic cell, including yeast and animal cells, particularly mammalian cells (e.g., CHO cells, NS0 cells, human cells such as PER.C6, HEK293 or HKB-11 cells, myeloma cells, or a human liver cell), as well as plant cells. In some embodiments, the transfected host cell is a mammalian cell, such as a human cell. In some embodiments, expression hosts can glycosylate the antibody of the invention, particularly with carbohydrate structures that are not themselves immunogenic in humans. In some embodiments the transfected host cell may be able to grow in serum-free media. In further embodiments the transfected host cell may be able to grow in culture without the presence of animal-derived products. The transfected host cell may also be cultured to give a cell line.

The invention also provides a method of preparing the antibody of interest comprising the steps of: culturing or sub-culturing a transfected host cell population, e.g. a stably transfected host cell population, under conditions where the antibody of interest is expressed and, optionally, purifying the antibody of interest. The transfected host cell population may be prepared by (i) providing nucleic acid(s) encoding a selected antibody of interest, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest.

In some embodiments, antibodies according to the invention may be produced by (i) expressing a nucleic acid sequence according to the invention in a host cell, e.g. by use of a vector (or host cell) according to the present invention, and (ii) isolating the expressed antibody product. Additionally, the method may include (iii) purifying the isolated antibody.

Accordingly, after production, the antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

### Compositions

The present invention also provides a composition comprising one or more of:
(i) the antibody of the present invention, or an antigen-binding fragment thereof;
(ii) the nucleic acid or the plurality of nucleic acids of the present invention;
(iii) the vector or the plurality of vectors of the present invention; or
(iv) the cell expressing the antibody according to the present invention or comprising the vector according to the present invention.

The composition may be used for treatment or diagnostic purposes. Accordingly, the composition may be a pharmaceutical composition or a diagnostic composition. The composition may comprise a (pharmaceutically acceptable) excipient, diluent or carrier.

Accordingly, the present invention also provides a pharmaceutical composition comprising the antibody according to the present invention, or an antigen-binding fragment thereof, the nucleic acid or the plurality of nucleic acids of the present invention, the vector or the plurality of vectors of the present invention, and/or the cell according to the present invention.

The pharmaceutical composition may optionally also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In some embodiments, the pharmaceutically acceptable carrier, diluent and/or excipient in the pharmaceutical composition is not an active component in respect to birch pollen allergy or related allergy.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin®, for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody may be provided in kit form with sterile water or a sterile buffer.

In some embodiments, the (only) active ingredient in the composition is the antibody as described herein. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

The present invention also provides a method of preparing a pharmaceutical composition comprising the steps of: (i) preparing an antibody of the invention; and (ii) admixing the purified antibody with one or more pharmaceutically acceptable excipients, diluents or carriers.

In other embodiments, a method of preparing a pharmaceutical composition comprises the step of: admixing an antibody with one or more pharmaceutically-acceptable carriers, wherein the antibody is a monoclonal antibody.

Pharmaceutical compositions may generally have a pH between 5.5 and 8.5, in some embodiments this may be between 6 and 8, for example about 7. The pH may be maintained by the use of a buffer. The composition may be sterile and/or pyrogen free. The composition may be isotonic with respect to humans. In some embodiments pharmaceutical compositions are supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody may be in dry form, for reconstitution before use with an appropriate sterile liquid.

A vehicle is typically understood to be a material that is suitable for storing, transporting, and/or administering a compound, such as a pharmaceutically active compound, in particular the antibodies as described herein. For example, the vehicle may be a physiologically acceptable liquid, which is suitable for storing, transporting, and/or administering a pharmaceutically active compound, in particular the antibodies as described herein. Once formulated, the compositions can be administered directly to the subject. In some embodiments the compositions are adapted for administration to mammalian, e.g., human subjects.

Pharmaceutical compositions may include an antimicrobial, particularly if packaged in a multiple dose format. They may comprise detergent e.g., a Tween (polysorbate), such as Tween 80. Detergents are generally present at low levels e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The pharmaceutical compositions may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Optionally, the pharmaceutical composition may be prepared for oral administration, e.g. as tablets, capsules and the like, for topical administration, or as injectable, e.g. as liquid solutions or suspensions. In some embodiments, the pharmaceutical composition is an injectable. Solid forms suitable for solution in, or suspension in, liquid vehicles prior to injection are also encompassed, for example the pharmaceutical composition may be in lyophilized form.

For injection, e.g. intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is an antibody, a peptide, a nucleic acid molecule, or another pharmaceutically useful compound that is to be given to an individual, administration is usually in an “effective amount”, e.g. in a “prophylactically effective amount” or a “therapeutically effective amount” (as the case may be), this being sufficient to show benefit to the individual. The actual amount administered, and rate and time-course of administration, will depend on the nature and severity of what is being treated. For injection, the pharmaceutical composition may be provided for example in a pre-filled syringe.

The pharmaceutical composition may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the antibody as defined above, is combined with emulsifying and suspending agents. If desired, certain sweetening, flavoring or coloring agents may also be added.

The pharmaceutical composition may also be administered topically, especially when the target of treatment includes areas or organs readily accessible by topical application, e.g. including accessible epithelial tissue. Suitable topical formulations are readily prepared for each of these areas or organs. For topical applications, the pharmaceutical composition may be formulated in a suitable ointment, containing the pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutical composition can be formulated in a suitable lotion or cream. Suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule. For a single dose, e.g. a daily, weekly or monthly dose, the amount of the antibody in the pharmaceutical composition, may not exceed 1 g or 500 mg. In some embodiments, for a single dose, the amount of the antibody in the pharmaceutical composition, may not exceed 200 mg, or 100 mg. For example, for a single dose, the amount of the antibody in the pharmaceutical composition, may not exceed 50 mg.

In some embodiments, the composition may include antibodies of the invention, wherein the antibodies may make up at least 50% by weight (e.g., 60%, 70%, 75%, 80%, 85%, 90%, 95%, 96%, 97%, 98%, 99% or more) of the total protein in the composition. In the composition, the antibodies may be in purified form.

As an alternative to delivering antibodies for therapeutic purposes, it is possible to deliver nucleic acid (typically DNA) that encodes the monoclonal antibody of interest to a subject, such that the nucleic acid can be expressed in the subject in situ to provide a desired therapeutic effect. Suitable gene therapy and nucleic acid delivery vectors are known in the art.

Pharmaceutical compositions typically include an “effective” amount of one or more antibodies as described herein, i.e. an amount that is sufficient to treat, ameliorate, attenuate, decrease or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. An effective dose may generally be from about 0.005 to about 100 mg/kg, for example from about 0.0075 to about 50 mg/kg or from about 0.01 to about 10 mg/kg. In some embodiments, the effective dose will be from about 0.02 to about 5 mg/kg, of the antibody (e.g. amount of the antibody in the pharmaceutical composition) in relation to the bodyweight (e.g., in kg) of the individual to which it is administered.

Moreover, the pharmaceutical composition may also comprise an additional active component, which may be a further antibody or a component, which is not an antibody. In other embodiments, the pharmaceutical composition may not comprise an additional active component (in addition to the antibody of the invention or respective nucleic acids, vectors or cells as described above).

Accordingly, the pharmaceutical composition may comprise one or more of the additional active components. The antibody of the invention can be present either in the same pharmaceutical composition as the additional active component or, alternatively, the antibody may be comprised by a first pharmaceutical composition and the additional active component may be comprised by a second pharmaceutical composition different from the first pharmaceutical composition. Accordingly, if more than one additional active component is envisaged, each additional active component and the antibody may be comprised in a different pharmaceutical composition. Such different pharmaceutical compositions may be administered either combined/simultaneously or at separate times or at separate locations (e.g. separate parts of the body), optionally by different routes of administration.

The antibody and the additional active component may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

Preferably, the composition comprises at least two distinct antibodies, or antigen-binding fragments thereof, binding to distinct, non-overlapping epitopes of Bet v1, and optionally binding to distinct, non-overlapping epitopes of a Bet v1 homologous protein. More preferably, the composition comprises at least three distinct antibodies, or antigen-binding fragments thereof, binding to distinct, non-overlapping epitopes of Bet v1, and optionally binding to distinct, non-overlapping epitopes of a Bet v1 homologous protein. As shown in the appended examples, 4E1, 25-3D3 and 17B10 bind to distinct, non-overlapping epitopes of Bet v 1. Further, it has been shown by cross-competition ELISA on Bet v 1 that antibodies 17B10, 6C9, 3F1 and 13A2 belong to the same complementation group, and therefore it can be assumed that 17B10, 6C9, 3F1 and 13A2 bind to the same epitope on Bet v 1.

Accordingly, the composition may comprise at least two antibodies or antigen-binding fragments thereof, selected from the group consisting of:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above; and an antibody, or an antigen-binding fragment thereof, according to any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B110, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 13A2, or sequence variants thereof, as described above.

In some embodiments, the composition comprises (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above; and
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above.

In some embodiments, the composition comprises (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above; and an antibody selected from any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B110, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 17B110, or sequence variants thereof, as described above.

In some embodiments, the composition comprises (exactly) two distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above; and an antibody, or an antigen-binding fragment thereof, selected from any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B10, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 17B110, or sequence variants thereof, as described above.

Preferably, the composition comprises at least three distinct antibodies, or antigen-binding fragments, binding to distinct, non-overlapping epitopes of Bet v 1, wherein the antibodies, or antigen-binding fragments thereof, are selected from the group consisting of:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above; and an antibody, or an antigen-binding fragment thereof, according to any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B10, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 17B10, or sequence variants thereof, as described above.

The present invention also provides a composition comprising at least two distinct antibodies, or antigen-binding fragments thereof, wherein the at least two distinct antibodies or antigen-binding fragments bind to distinct, non-overlapping epitopes of Bet v 1, and optionally bind to distinct, non-overlapping epitopes of a Bet v 1 homologous protein, wherein the composition comprises at least two antibodies, or antigen-binding fragments thereof, selected from the group consisting of:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14; and an antibody, or an antigen-binding fragment thereof, according to any one of (iii) to (vi):
(iii) an antibody, or an antigen-binding fragment thereof, comprising a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antibody, or an antigen-binding fragment thereof, comprising a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at least 70% identity to SEQ ID NO: 70, and a CDRL3 having at least 70% identity to SEQ ID NO: 71;
(v) an antibody, or an antigen-binding fragment thereof, comprising a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79;
(vi) an antibody, or an antigen-binding fragment thereof, comprising a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87.

Preferably, the composition comprises at least two antibodies, or antigen-binding fragments thereof, selected from the group consisting of:
(i) an antibody, or an antigen-binding fragment thereof, comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14; and an antibody, or an antigen-binding fragment thereof, according to any one of (iii) to (vi):
(iii) an antibody, or an antigen-binding fragment thereof, comprising a CDRH1 according to SEQ ID NO: 17, a CDRH2 according to SEQ ID NO: 18, a CDRH3 according to SEQ ID NO: 19, a CDRL1 according to SEQ ID NO: 20, a CDRL2 according to SEQ ID NO: 21, and a CDRL3 according to SEQ ID NO: 22;
(iv) an antibody, or an antigen-binding fragment thereof, comprising a CDRH1 according to SEQ ID NO: 66, a CDRH2 according to SEQ ID NO: 67, a CDRH3 according to SEQ ID NO: 68, a CDRL1 according to SEQ ID NO: 69, a CDRL2 according to SEQ ID NO: 70, and a CDRL3 according to SEQ ID NO: 71;
(v) an antibody, or an antigen-binding fragment thereof, comprising a CDRH1 according to SEQ ID NO: 74, a CDRH2 according to SEQ ID NO: 75, a CDRH3 according to SEQ ID NO: 76, a CDRL1 according to SEQ ID NO: 77, a CDRL2 according to SEQ ID NO: 78, and a CDRL3 according to SEQ ID NO: 79;
(vi) an antibody, or an antigen-binding fragment thereof, comprising a CDRH1 according to SEQ ID NO: 82, a CDRH2 according to SEQ ID NO: 83, a CDRH3 according to SEQ ID NO: 84, a CDRL1 according to SEQ ID NO: 85, a CDRL2 according to SEQ ID NO:86, and a CDRL3 according to SEQ ID NO: 87.

More preferably, the composition comprises at least two antibodies, or antigen-binding fragments thereof, selected from the group consisting of:
(i) an antibody, or an antigen-binding fragment thereof, comprising a VH having at least 70% identity to SEQ ID NO: 7; and a VL having at least 70% identity to SEQ ID NO: 8;
(ii) an antibody, or an antigen-binding fragment thereof, comprising a VH having at least 70% identity to SEQ ID NO: 15; and a VL having at least 70% identity to SEQ ID NO: 16; and an antibody, or an antigen-binding fragment thereof, according to any one of (iii) to (vi):
(iii) an antibody, or an antigen-binding fragment thereof, comprising a VH having at least 70% identity to SEQ ID NO: 23; and a VL having at least 70% identity to SEQ ID NO: 24,
(iv) an antibody, or an antigen-binding fragment thereof, comprising a VH having at least 70% identity to SEQ ID NO: 72; and a VL having at least 70% identity to SEQ ID NO: 73;
(v) an antibody, or an antigen-binding fragment thereof, comprising a VH having at least 70% identity to SEQ ID NO: 80; and a VL having at least 70% identity to SEQ ID NO: 81;
(vi) an antibody, or an antigen-binding fragment thereof, comprising a VH having at least 70% identity to SEQ ID NO: 88; and a VL having at least 70% identity to SEQ ID NO: 89.

Even more preferably, the composition comprises at least two antibodies, or antigen-binding fragments thereof, selected from the group consisting of:
(i) an antibody, or an antigen-binding fragment thereof, comprising a VH according to SEQ ID NO: 7; and a VL according to SEQ ID NO: 8;
(ii) an antibody, or an antigen-binding fragment thereof, comprising a VH according to SEQ ID NO: 15; and a VL according to SEQ ID NO: 16; and an antibody, or an antigen-binding fragment thereof, according to any one of (iii) to (vi):
(iii) an antibody, or an antigen-binding fragment thereof, comprising a VH according to SEQ ID NO: 23; and a VL according to SEQ ID NO: 24;
(iv) an antibody, or an antigen-binding fragment thereof, comprising a VH according to SEQ ID NO: 72; and a VL according to SEQ ID NO: 73;
(v) an antibody, or an antigen-binding fragment thereof, comprising a VH according to SEQ ID NO: 80; and a VL according to SEQ ID NO: 81;
(vi) an antibody, or an antigen-binding fragment thereof, comprising a VH according to SEQ ID NO: 88; and a VL according to SEQ ID NO: 89.

Preferably, the composition comprises at least three antibodies, or antigen-binding fragments thereof, selected from the group consisting of:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to SEQ ID NO: 7; and a VL having at least 70% identity to SEQ ID NO: 8; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to SEQ ID NO: 7; and a VL according to SEQ ID NO: 8;
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NO: 15; and a VL having at least 70% identity to SEQ ID NO: 16; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to SEQ ID NO: 15; and a VL according to SEQ ID NO: 16;
and an antibody, or an antigen-binding fragment thereof, according to any one of (iii) to (vi):
(iii) an antibody, or an antigen-binding fragment thereof, comprising:
   a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to SEQ ID NO: 17, a CDRH2 according to SEQ ID NO: 18, a CDRH3 according to SEQ ID NO: 19, a CDRL1 according to SEQ ID NO: 20, a CDRL2 according to SEQ ID NO: 21, and a CDRL3 according to SEQ ID NO: 22;
   in some embodiments a VH having at least 70% identity to SEQ ID NO: 23; and a VL having at least 70% identity to SEQ ID NO: 24; wherein the CDRH1 according SEQ ID NO: 17, CDRH2 according to SEQ ID NO: 18, CDRH3 according to SEQ ID NO: 19, CDRL1 according to SEQ ID NO: 20, CDRL2 according to SEQ ID NO: 21, and CDRL3 according to SEQ ID NO: 22 are preferably maintained;
   such as a VH according to SEQ ID NO: 23; and a VL according to SEQ ID NO: 24;
(iv) an antibody, or an antigen-binding fragment thereof, comprising:
   a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at
   least 70% identity to SEQ ID NO: 70, and a CDRL3 having at least 70% identity to SEQ ID NO: 71;
   in particular a CDRH1 according to SEQ ID NO: 66, a CDRH2 according to SEQ ID NO: 67, a CDRH3 according to SEQ ID NO: 68, a CDRL1 according to SEQ ID NO: 69, a CDRL2 according to SEQ ID NO: 70, and a CDRL3 according to SEQ ID NO: 71;
   in some embodiments a VH having at least 70% identity to SEQ ID NO: 72; and a VL having at least 70% identity to SEQ ID NO: 73; wherein the CDRH1 according SEQ ID NO: 66, CDRH2 according to SEQ ID NO: 67, CDRH3 according to SEQ ID NO: 68, CDRL1 according to SEQ ID NO: 69, CDRL2 according to SEQ ID NO: 70, and CDRL3 according to SEQ ID NO: 71 are preferably maintained;
   such as a VH according to SEQ ID NO: 72; and a VL according to SEQ ID NO: 73;
(v) an antibody, or an antigen-binding fragment thereof, comprising:
   a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79;
   in particular a CDRH1 according to SEQ ID NO: 74, a CDRH2 according to SEQ ID NO: 75, a CDRH3 according to SEQ ID NO: 76, a CDRL1 according to SEQ ID NO: 77, a CDRL2 according to SEQ ID NO: 78, and a CDRL3 according to SEQ ID NO: 79;
   in some embodiments a VH having at least 70% identity to SEQ ID NO: 80; and a VL having at least 70% identity to SEQ ID NO: 81; wherein the CDRH1 according SEQ ID NO: 74, CDRH2 according to SEQ ID NO: 75, CDRH3 according to SEQ ID NO: 76, CDRL1 according to SEQ ID NO: 77, CDRL2 according to SEQ ID NO: 78, and CDRL3 according to SEQ ID NO: 79 are preferably maintained;
   such as a VH according to SEQ ID NO: 80; and a VL according to SEQ ID NO: 81;
(vi) an antibody, or an antigen-binding fragment thereof, comprising:
   a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87;
   in particular a CDRH1 according to SEQ ID NO: 82, a CDRH2 according to SEQ ID NO: 83, a CDRH3 according to SEQ ID NO: 84, a CDRL1 according to SEQ ID NO: 85, a CDRL2 according to SEQ ID NO: 86, and a CDRL3 according to SEQ ID NO: 87;
   in some embodiments a VH having at least 70% identity to SEQ ID NO: 88; and a VL having at least 70% identity to SEQ ID NO: 89; wherein the CDRH1 according SEQ ID NO: 82, CDRH2 according to SEQ ID NO: 83, CDRH3 according to SEQ ID NO: 84, CDRL1 according to SEQ ID NO: 85, CDRL2 according to SEQ ID NO: 86, and CDRL3 according to SEQ ID NO: 87 are preferably maintained;
   such as a VH according to SEQ ID NO: 88; and a VL according to SEQ ID NO: 89.

Preferably, the composition comprises antibodies binding to three distinct, non-overlapping epitopes on Bet v 1. Accordingly, it is preferred that the composition comprises at least three distinct antibodies, or antigen-binding fragments, and preferably comprises an antibody, or an antigen-binding fragment thereof, according to (i), an antibody, or an antigen-binding fragment thereof, according to (ii), and an antibody, or an antigen-binding fragment thereof, selected from at least one of (iii) to (vi), as defined above.

In another preferred embodiment, the composition comprises an antibody, or an antigen-binding fragment thereof, according to (i); an antibody, or an antigen-binding fragment thereof, according to (ii); and an antibody, or an antigen-binding fragment thereof, selected from at least one of (iv) to (vi). As shown in the examples below, antibodies as defined in (iv), (v) and (vi) above, exemplified by antibodies 6C9, 3F1 and 13A2, belonging to the same complementation group as an antibody as defined in (iii) above, exemplified by 17B110, possess cross-reactivity to oak antigens and also bind to food related walnut antigens.

Accordingly, the antibodies comprised in the composition of the present invention may be selected in such a way that the desired allergens in particular are targeted. If, for example, oak pollen is to be targeted in addition to an effect against birch pollen, it is advantageous that the composition comprises two antibodies that are particularly effective against oak pollen. This can be achieved, for example, by combining an antibody according to (i) above (e.g. 4E1) with an antibody selected from any one of (iv) to (vi) above (e.g. 6C9, 3F1, 13A2).

As described above, the composition of the invention preferably comprises antibodies binding to at least two, preferably three, distinct non-overlapping epitopes of Bet v 1. The antibodies, or the antigen-binding fragments thereof, which are comprised in the composition, may be selected from at least two or three different antibodies of 4E1, 25-3D3, 17B110, 6C9, 3F1 and 13A2, or variants thereof, as described above, in particular as shown in Tables 1 and 2. Preferably, the composition therefore comprises two or three (monospecific) antibodies as described herein, in particular selected from the different antibodies 4E1, 25-3D3, 17B10, 6C9, 3F1 and 13A2, or the variants thereof, as described above, in particular as shown in Tables 1 and 2. In some embodiments, the composition comprises multispecific antibodies, in particular as described herein.

Binding to three different epitopes of Bet v 1 may be accomplished by compositions including:
(i) three distinct monospecific antibodies, or antigen-binding fragments thereof, binding to three distinct epitopes of Bet v 1, in particular selected from 4E1, 25-3D3, 17B10, 6C9, 3F1 and 13A2, or variants thereof, as described above, in particular as shown in Tables 1 and 2;
(ii) (1) a bispecific antibody, or a bispecific antigen-binding fragment, in particular having two specificities selected from 4E1, 25-3D3, 17B10, 6C9, 3F1 and 13A2, or variants thereof, as described above, in particular as shown in Tables 1 and 2, and (2) an additional monospecific antibody, in particular selected from 4E1, 25-3D3, 17B110, 6C9, 3F1 and 13A2, or variants thereof, as described above, in particular as shown in Tables 1 and 2, binding to another epitope on Bet v 1; or
(iii) a trispecific antibody, or a trispecific antigen-binding fragment, in particular having three specificities selected from 4E1, 25-3D3, 17B10, 6C9, 3F1 and 13A2, or variants thereof, as described above, in particular as shown in Tables 1 and 2.

Alternatively or additionally, the composition may further comprise at least one additional agent useful for treating a birch pollen allergy and/or a related allergy, e.g. a tree pollen allergy, such as hazel pollen allergy, black alder pollen allergy, beech pollen allergy, oak tree pollen allergy, chestnut pollen allergy, hop-hornbeam pollen allergy and hornbeam pollen allergy, and/or a related food allergy, such as apple allergy, hazelnut allergy and walnut allergy. The additional agent useful for treating the above allergies may be selected from the group comprising: a β-adrenergic agonist (e.g. epinephrine), antihistamine, a corticosteroid, an anti-IgE antibody, an anti-IgE antibody binding fragment, a peptide vaccine and further antibodies capable of binding to a birch pollen allergen and/or related (tree pollen and/or food) allergen. In some embodiments, the composition comprises a β-adrenergic agonist, such as epinephrine.

In some embodiments, the composition comprises a birch pollen allergen and/or a related (tree pollen and/or food) allergen. In some embodiments, the composition comprises an extract comprising a birch pollen allergen and/or a related (tree pollen and/or food) allergen. The birch pollen allergen may be a birch pollen protein, such as Bet v 1. In some embodiments, the composition may comprise a bet v 1 homologous protein, e.g. a hazel pollen allergen, such as Cor a 1, a black alder pollen allergen, such as Aln g 1, a beech pollen allergen, such as Fag s 1, an oak tree pollen allergen, such as Que a 1, a chestnut pollen allergen, such as Cas s 1, a hop-hornbeam pollen allergen, such as Ost c 1, a hornbeam pollen allergen, such as Car b1, and/or a related food allergen, e.g. an apple allergen, such as Mal d 1, a hazelnut allergen, such as Cor a 1, and/or and walnut allergen, such as Jug r 5, or any other Bet v 1 homologous protein mentioned above. The birch pollen allergen or related (tree pollen or food) allergen may be of natural origin, recombinantly expressed or may be a synthetic peptide. In some embodiments, the anti-bet v 1 allergen antibodies, or antigen-binding fragments thereof, as described herein, may be pre-incubated with the birch pollen allergen or related (tree pollen or food) allergen and may be administered as a mixture to a subject.

Combining a birch pollen allergen or related (tree pollen or food) allergen, for example selected from the group consisting of Bet v 1, Cor a 1, Aln g 1, Fag s 1, Que a 1, Cas s 1, Ost c 1, Car b1, Mal d 1, and Jug r 5 or a mixture thereof, or any other Bet v 1 homologous protein mentioned above, or an extract comprising a birch pollen allergen or related (tree pollen or food) allergen, as exemplified above, with the antibodies of the invention increases the safety of administering the allergen (e.g. in desensitization). Furthermore, without being bound to any theory, the present inventors assume that the combination of antibodies with the respective allergen (targeted by the antibodies), i.e. the combination of passive (antibodies) and active (allergen) immunization, has a synergistic effect. The components, i.e. the allergen component and the antibody component, may be administered simultaneously or in a timely staggered manner, e.g. by administering the components separately from one another, e.g. within 15, 30, 60 or 90 min.

The present invention also provides a diagnostic composition comprising an antibody according to the present invention, a nucleic acid(s) according to the present invention, a vector(s) according to the present invention, and/or a cell according to the present invention. The diagnostic composition may optionally comprise suitable means for detection, such as reagents conventionally used in immuno- or nucleic acid based diagnostic methods.

The antibodies described herein are, for example, suited for diagnostic purposes. Accordingly, they may be used in immunoassays, in which they can be utilized in liquid phase or bound to a solid phase carrier. Such immunoassays may be competitive or non-competitive immunoassays; in either a direct or in an indirect format. Examples of such immunoassays include, but are not limited to, radioimmunoassay (RIA), enzyme-linked immunoassay (ELISA), sandwich (immunometric assay), immunohistochemistry, flow cytometry and Western blot assay. To this end, the antibody may be labelled, e.g. as described above.

### Kits

In a further aspect the present invention also provides a kit comprising one or more of
(i) the antibody according to the present invention, or an antigen-binding fragment thereof, as described above,
(ii) the nucleic acid molecule (or the plurality of nucleic acid molecules) according to the present invention as described above,
(iii) the vector (or the plurality of vectors) according to the present invention as described above,
(iv) the cell according to the present invention as described above, and/or
(v) the composition according to the present invention as described above.

In addition, the kit may comprise means for administration of the antibody, or an antigen binding fragment thereof, according to the present invention, the nucleic acid according to the present invention, the vector according to the present invention, the cell according to the present invention or the pharmaceutical composition according to the present invention, such as a syringe or a vessel, a leaflet, and/or a co-agent to be administered as described herein. For example, the kit may contain a leaflet, e.g. comprising instructions for use. In addition or alternatively, the kit may comprise one or more reagents, e.g. for use in appropriate diagnostic assays. In some instances, the kit may contain a reference agent or control. In some embodiments, the composition of the invention may be provided in kit form, e.g., designed such that a combined composition is reconstituted just prior to administration to a subject. For example, a lyophilized antibody may be provided in kit form with sterile water or a sterile buffer (e.g., in a separate container).

In some embodiments, the kit comprises at least two distinct antibodies (or nucleic acids encoding such antibodies or compositions comprising such antibodies), wherein the distinct antibodies may be provided in distinct containers. Preferably, the kit comprises at least two distinct antibodies, or antigen-binding fragments thereof, binding to distinct, non-overlapping epitopes of Bet v 1, and optionally binding to distinct, non-overlapping epitopes of at least one Bet v 1 homologous protein (or nucleic acids encoding such antibodies or compositions comprising such antibodies). More preferably, the kit comprises at least three distinct antibodies, or antigen-binding fragments thereof, binding to distinct, non-overlapping epitopes of Bet v 1, and optionally binding to distinct, non-overlapping epitopes of at least one Bet v 1 homologous protein (or nucleic acids encoding such antibodies or compositions comprising such antibodies). To this end, the antibodies contained in the kit may be selected from the antibodies, or variants thereof, as described above, e.g. in Tables 1 and 2. As shown in the appended examples, antibodies according to the present invention may bind to distinct, non-overlapping epitopes of Bet v 1.

Accordingly, the kit may comprise
(i) at least two distinct antibodies, or antigen-binding fragments thereof, according to the present invention, as described above, which bind to distinct, non-overlapping epitopes of Bet v 1, and optionally bind to distinct, non-overlapping epitopes of at least one Bet v 1 homologous protein;
(ii) nucleic acid molecule(s) according to the present invention as described above, which encode at least two distinct antibodies, or antigen-binding fragments thereof, which bind to distinct, non-overlapping epitopes of Bet v 1, and optionally bind to distinct, non-overlapping epitopes of at least one Bet v 1 homologous protein; or
(iii) composition(s) comprising (i) or (ii).

While the following description refers to kits comprising distinct antibodies, or antigen-binding fragments thereof, it is understood that the kits may likewise comprise (distinct) nucleic acid(s) encoding said antibodies, or antigen-binding fragments; or (distinct) composition(s) comprising said antibodies, or antigen-binding fragments, or said nucleic acid(s).

In particular, the kit may comprise at least two of the following:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above; and an antibody, or an antigen-binding fragment thereof, according to any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B110, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 13A2, or sequence variants thereof, as described above.

In some embodiments, the kit may comprise (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above; and
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above.

In some embodiments, the kit may comprise (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above; and an antibody, or an antigen-binding fragment thereof, selected from any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B10, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 13A2, or sequence variants thereof, as described above.

In some embodiments, the kit may comprise (exactly) two distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above; and an antibody, or an antigen-binding fragment thereof, selected from any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B110, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 13A2, or sequence variants thereof, as described above.

Preferably, the kit comprises at least three distinct antibodies, or antigen-binding fragments thereof, which bind to distinct, non-overlapping epitopes of Bet v 1 and optionally bind to distinct, non-overlapping epitopes of a Bet v 1 homologous protein; or nucleic acid(s) encoding said antibodies or compositions comprising said antibodies.

Therefore, in a preferred embodiment, the kit may comprise (exactly) three distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above; and an antibody, or an antigen-binding fragment thereof, selected from any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B10, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 13A2, or sequence variants thereof, as described above.

The present invention also provides a kit comprising at least two distinct antibodies, or antigen-binding fragments thereof, wherein the two distinct antibodies or antigen-binding fragments bind to distinct, non-overlapping epitopes of Bet v 1, and optionally bind to distinct, non-overlapping epitopes of at least one Bet v 1 homologous protein, wherein the kit comprises at least two of the following:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14; and an antibody, or an antigen-binding fragment thereof, according to any one of (iii) to (vi):
(iii) an antibody, or an antigen-binding fragment thereof, comprising a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antibody, or an antigen-binding fragment thereof, comprising a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at least 70% identity to SEQ ID NO: 70, and a CDRL3 having at least 70% identity to SEQ ID NO: 71;
(v) an antibody, or an antigen-binding fragment thereof, comprising a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79;
(vi) an antibody, or an antigen-binding fragment thereof, comprising a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87.

Preferably, the kit comprises at least two of the following:
(i) an antibody, or an antigen-binding fragment thereof, comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14; and an antibody, or an antigen-binding fragment thereof, according to any one of (iii) to (vi):
(iii) an antibody, or an antigen-binding fragment thereof, comprising a CDRH1 according to SEQ ID NO: 17, a CDRH2 according to SEQ ID NO: 18, a CDRH3 according to SEQ ID NO: 19, a CDRL1 according to SEQ ID NO: 20, a CDRL2 according to SEQ ID NO: 21, and a CDRL3 according to SEQ ID NO: 22;
(iv) an antibody, or an antigen-binding fragment thereof, comprising a CDRH1 according to SEQ ID NO: 66, a CDRH2 according to SEQ ID NO: 67, a CDRH3 according to SEQ ID NO: 68, a CDRL1 according to SEQ ID NO: 69, a CDRL2 according to SEQ ID NO: 70, and a CDRL3 according to SEQ ID NO: 71;
(v) an antibody, or an antigen-binding fragment thereof, comprising a CDRH1 according to SEQ ID NO: 74, a CDRH2 according to SEQ ID NO: 75, a CDRH3 according to SEQ ID NO: 76, a CDRL1 according to SEQ ID NO: 77, a CDRL2 according to SEQ ID NO: 78, and a CDRL3 according to SEQ ID NO: 79;
(vi) an antibody, or an antigen-binding fragment thereof, comprising a CDRH1 according to SEQ ID NO: 82, a CDRH2 according to SEQ ID NO: 83, a CDRH3 according to SEQ ID NO: 84, a CDRL1 according to SEQ ID NO: 85, a CDRL2 according to SEQ ID NO:86, and a CDRL3 according to SEQ ID NO: 87.

Preferably, the kit comprises at least two of the following:
(i) an antibody, or an antigen-binding fragment thereof, comprising a VH having at least 70% identity to SEQ ID NO: 7; and a VL having at least 70% identity to SEQ ID NO: 8;
(ii) an antibody, or an antigen-binding fragment thereof, comprising a VH having at least 70% identity to SEQ ID NO: 15; and a VL having at least 70% identity to SEQ ID NO: 16; and an antibody, or an antigen-binding fragment thereof, according to any one of (iii) to (vi):
(iii) an antibody, or an antigen-binding fragment thereof, comprising a VH having at least 70% identity to SEQ ID NO: 23; and a VL having at least 70% identity to SEQ ID NO: 24;
(iv) an antibody, or an antigen-binding fragment thereof, comprising a VH having at least 70% identity to SEQ ID NO: 72; and a VL having at least 70% identity to SEQ ID NO: 73;
(v) an antibody, or an antigen-binding fragment thereof, comprising a VH having at least 70% identity to SEQ ID NO: 80; and a VL having at least 70% identity to SEQ ID NO: 81;
(vi) an antibody, or an antigen-binding fragment thereof, comprising a VH having at least 70% identity to SEQ ID NO: 88; and a VL having at least 70% identity to SEQ ID NO: 89.

More preferably, the kit comprises at least two of the following:
(i) an antibody, or an antigen-binding fragment thereof, comprising a VH according to SEQ ID NO: 7; and a VL according to SEQ ID NO: 8;
(ii) an antibody, or an antigen-binding fragment thereof, comprising a VH according to SEQ ID NO: 15; and a VL according to SEQ ID NO: 16; and an antibody, or an antigen-binding fragment thereof, according to any one of (iii) to (vi):
(iii) an antibody, or an antigen-binding fragment thereof, comprising a VH according to SEQ ID NO: 23; and a VL according to SEQ ID NO: 24;
(iv) an antibody, or an antigen-binding fragment thereof, comprising a VH according to SEQ ID NO: 72; and a VL according to SEQ ID NO: 73;
(v) an antibody, or an antigen-binding fragment thereof, comprising a VH according to SEQ ID NO: 80; and a VL according to SEQ ID NO: 81;
(vi) an antibody, or an antigen-binding fragment thereof, comprising a VH according to SEQ ID NO: 88; and a VL according to SEQ ID NO: 89.

Therefore, the kit preferably comprises at least two of the following:
(i) an antibody, or an antigen-binding fragment thereof, comprising:
a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to SEQ ID NO: 7; and a VL having at least 70% identity to SEQ ID NO: 8; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to SEQ ID NO: 7; and a VL according to SEQ ID NO: 8
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to any one of SEQ ID NO: 15; and a VL having at least 70% identity to SEQ ID NO: 16; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to SEQ ID NO: 15; and a VL according to SEQ ID NO: 16; and an antibody, or an antigen-binding fragment thereof, according to any one of (iii) to (vi):
(iii) an antibody, or an antigen-binding fragment thereof, comprising:
   a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to SEQ ID NO: 17, a CDRH2 according to SEQ ID NO: 18, a CDRH3 according to SEQ ID NO: 19, a CDRL1 according to SEQ ID NO: 20, a CDRL2 according to SEQ ID NO: 21, and a CDRL3 according to SEQ ID NO: 22;
   in some embodiments a VH having at least 70% identity to SEQ ID NO: 23; and a VL having at least 70% identity to SEQ ID NO: 24; wherein the CDRH1 according SEQ ID NO: 17, CDRH2 according to SEQ ID NO: 18, CDRH3 according to SEQ ID NO: 19, CDRL1 according to SEQ ID NO: 20, CDRL2 according to SEQ ID NO: 21, and CDRL3 according to SEQ ID NO: 22 are preferably maintained;
   such as a VH according to SEQ ID NO: 23; and a VL according to SEQ ID NO: 24;
(iv) an antibody, or an antigen-binding fragment thereof, comprising:
   a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at least 70% identity to SEQ ID NO: 70, and a CDRL3 having at least 70% identity to SEQ ID NO: 71;
   in particular a CDRH1 according to SEQ ID NO: 66, a CDRH2 according to SEQ ID NO: 67, a CDRH3 according to SEQ ID NO: 68, a CDRL1 according to SEQ ID NO: 69, a CDRL2 according to SEQ ID NO: 70, and a CDRL3 according to SEQ ID NO: 71;
   in some embodiments a VH having at least 70% identity to SEQ ID NO: 72; and a VL having at least 70% identity to SEQ ID NO: 73; wherein the CDRH1 according SEQ ID NO: 66, CDRH2 according to SEQ ID NO: 67, CDRH3 according to SEQ ID NO: 68, CDRL1 according to SEQ ID NO: 69, CDRL2 according to SEQ ID NO: 70, and CDRL3 according to SEQ ID NO: 71 are preferably maintained;
   such as a VH according to SEQ ID NO: 72; and a VL according to SEQ ID NO: 73;
(v) an antibody, or an antigen-binding fragment thereof, comprising:
   a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79;
   in particular a CDRH1 according to SEQ ID NO: 74, a CDRH2 according to SEQ ID NO: 75, a CDRH3 according to SEQ ID NO: 76, a CDRL1 according to SEQ ID NO: 77, a CDRL2 according to SEQ ID NO: 78, and a CDRL3 according to SEQ ID NO: 79;
   in some embodiments a VH having at least 70% identity to SEQ ID NO: 80; and a VL having at least 70% identity to SEQ ID NO: 81; wherein the CDRH1 according SEQ ID NO: 74, CDRH2 according to SEQ ID NO: 75, CDRH3 according to SEQ ID NO: 76, CDRL1 according to SEQ ID NO: 77, CDRL2 according to SEQ ID NO: 78, and CDRL3 according to SEQ ID NO: 79 are preferably maintained;
   such as a VH according to SEQ ID NO: 80; and a VL according to SEQ ID NO: 81;
(vi) an antibody, or an antigen-binding fragment thereof, comprising:
   a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87;
   in particular a CDRH1 according to SEQ ID NO: 82, a CDRH2 according to SEQ ID NO: 83, a CDRH3 according to SEQ ID NO: 84, a CDRL1 according to SEQ ID NO: 85, a CDRL2 according to SEQ ID NO: 86, and a CDRL3 according to SEQ ID NO: 87;
   in some embodiments a VH having at least 70% identity to SEQ ID NO: 88; and a VL having at least 70% identity to SEQ ID NO: 89; wherein the CDRH1 according SEQ ID NO: 82, CDRH2 according to SEQ ID NO: 83, CDRH3 according to SEQ ID NO: 84, CDRL1 according to SEQ ID NO: 85, CDRL2 according to SEQ ID NO: 86, and CDRL3 according to SEQ ID NO: 87 are preferably maintained;
   such as a VH according to SEQ ID NO: 88; and a VL according to SEQ ID NO: 89.

Most preferably, the kit comprises at least three of the above (i), (ii) and (iii)-(vi). In a preferred embodiment, the kit comprises at least three antibodies, or antigen-binding fragments thereof, and preferably comprises an antibody, or an antigen-binding fragment thereof, according to (i), an antibody, or an antigen-binding fragment thereof, according to (ii), and an antibody, or an antigen-binding fragment thereof, according to at least one of (iii) to (vi), as defined above. In another preferred embodiment, the kit comprises an antibody, or an antigen-binding fragment thereof, according to (i), an antibody, or an antigen-binding fragment thereof, according to (ii), and an antibody, or an antigen-binding fragment thereof, according to at least one of (iv) to (vi), as defined above.

In particular, the antibodies comprised in the kit of the present invention may be selected in such a way that the desired allergens are targeted. If, for example, oak pollen is to be targeted in addition to an effect against birch pollen, it is advantageous that the kit contains two antibodies that are particularly effective against oak pollen. This can be achieved, for example, by combining an antibody according to (i) above (e.g. 4E1) with an antibody selected from any one of (iv) to (vi) above (e.g. 6C9, 3F1, 13A2).

As described above, the kit of the invention preferably comprises antibodies binding to at least two, preferably three, distinct non-overlapping epitopes of Bet v 1, and optionally binding to at least two, preferably three, distinct non-overlapping epitopes of a Bet v 1 homologous protein. The antibodies, or the antigen-binding fragments thereof, which are included in the composition, may be selected from the antibodies 4E1, 25-3D3, 17B110, 6C9, 3F1 and 13A2 or variants thereof, as described above, in particular as shown in Tables 1 and 2.

Preferably, the kit therefore comprises at least two or three (monospecific) antibodies as described herein, in particular selected from the antibodies 4E1, 25-3D3, 17B10, 6C9, 3F1 and 13A2, or variants thereof, as described above, in particular as shown in Tables 1 and 2. In some embodiments, the kit comprises multispecific antibodies, in particular as described herein.

Binding to three different epitopes of Bet v 1 may be accomplished by kits including:
(i) three distinct monospecific antibodies, or antigen-binding fragments thereof, binding to three distinct epitopes of Bet v 1, and optionally binding to at least two, preferably three, distinct non-overlapping epitopes of a Bet v 1 homologous protein, in particular selected from 4E1, 25-3D3, 17B10, 6C9, 3F1 and 13A2 or variants thereof, as described above, in particular as shown in Tables 1 and 2;
(ii) (1) a bispecific antibody, or a bispecific antigen-binding fragment, in particular having two specificities selected from 4E1, 25-3D3, 17B10, 6C9, 3F1 and 13A2 or variants thereof, as described above, in particular as shown in Tables 1 and 2, and (2) an additional monospecific antibody, in particular selected from 4E1, 25-3D3, 17B10, 6C9, 3F1 and 13A2 or variants thereof, as described above, in particular as shown in Tables 1 and 2, binding to another epitope on Bet v 1 (and optionally on a Bet v 1 homologous protein); or
(iii) a trispecific antibody, or a trispecific antigen-binding fragment, in particular having three specificities selected from 4E1, 25-3D3, 17B10, 6C9, 3F1 and 13A2 or variants thereof, as described above, in particular as shown in Tables 1 and 2.

Alternatively or additionally, the kit may further comprise at least one additional agent useful for treating a birch pollen allergy and/or a related allergy, e.g. a tree pollen allergy, such as hazel pollen allergy, black alder pollen allergy, beech pollen allergy, oak tree pollen allergy, chestnut pollen allergy, hop-hornbeam pollen allergy and hornbeam pollen allergy, and/or a related food allergy, such as apple allergy, hazelnut allergy and walnut allergy. The additional agent useful for treating the above allergies may be selected from the group comprising: a β-adrenergic agonist (e.g. epinephrine), antihistamine, a corticosteroid, an anti-IgE antibody, an anti-IgE antibody binding fragment, a peptide vaccine and further antibodies capable of binding to a birch pollen allergen and/or related (tree pollen and/or food) allergen. In some embodiments, the kit comprises a β-adrenergic agonist, such as epinephrine.

In some embodiments, the kit comprises a birch pollen allergen and/or a related allergen. The birch pollen allergen may be a birch pollen protein, such as Bet v 1. In some embodiments the kit may comprise a bet v 1 homologous tree pollen allergen, e.g. a hazel pollen allergen, such as Cor a 1, a black alder pollen allergen, such as Aln g 1, a beech pollen allergen, such as Fag s 1, an oak tree pollen allergen, such as Que a 1, a chestnut pollen allergen, such as Cas s 1, a hop-hornbeam pollen allergen, such as Ost c 1, and a hornbeam pollen allergen, such as Car b1, and/or a related food allergen, e.g. an apple allergen, such as Mal d 1, a hazelnut allergen, such as Cor a 1, and walnut allergen, such as Jug r 5, and/or any other Bet v 1 homologous allergen known in the art or mentioned herein. The birch pollen allergen and/or related (tree pollen or food) allergen may be of natural origin, recombinantly expressed or may be a synthetic peptide. In some embodiments, the antibodies, or antigen-binding fragments thereof, as described herein, may be provided (in a separate container) with instructions for pre-incubation with the birch pollen allergen and/or related (tree pollen or food) allergen before it is administered as a mixture to a subject. In other embodiments, the antibodies, or antigen-binding fragments thereof, as described herein, may be provided (in a separate container) with instructions for separate administration with the birch pollen allergen and/or related (tree pollen or food) allergen in a combined treatment schedule.

### Medical Treatments and Other Uses

In a further aspect, the present invention provides the use of the antibody according to the present invention, or an antigen-binding fragment thereof, the nucleic acid molecule (or the plurality of nucleic acid molecules) according to the present invention, the vector (or the plurality of vectors) according to the present invention, the cell according to the present invention, the (pharmaceutical) composition according to the present invention, or the kit according to the present invention as a medicament.

In particular, the antibody according to the present invention, or an antigen-binding fragment thereof, the nucleic acid molecule (or the plurality of nucleic acid molecules) according to the present invention, the vector (or the plurality of vectors) according to the present invention, the cell according to the present invention, the (pharmaceutical) composition according to the present invention, or the kit according to the present invention may be used in prophylaxis and/or treatment of a birch pollen allergy and/or related tree pollen allergy, which is preferably selected from at least one of hazel pollen allergy, black alder pollen allergy, beech pollen allergy, oak tree pollen allergy, chestnut pollen allergy, hop-hornbeam pollen allergy and hornbeam pollen allergy, and/or a related food allergy, which is preferably selected from at least one of apple allergy, hazelnut allergy and walnut allergy, or of a symptom of a birch pollen allergy and/or related (tree pollen and/or food) allergy, such as an anaphylactic reaction due to a birch pollen allergy or related (tree pollen and/or food).

Accordingly, the present invention also provides a method of treating, ameliorating or reducing a birch pollen allergy and/or related tree pollen allergy, which is preferably selected from at least one of hazel pollen allergy, black alder pollen allergy, beech pollen allergy, oak tree pollen allergy, chestnut pollen allergy, hop-hornbeam pollen allergy and hornbeam pollen allergy, and/or a related food allergy, which is preferably selected from at least one of apple allergy, hazelnut allergy and walnut allergy, or of a symptom of a birch pollen allergy and/or related allergy, such as an anaphylactic reaction due to a birch pollen allergy or related allergy, for example as described above, comprising: administering to a subject (in need thereof), (a therapeutically effective amount of) an antibody, or an antigen-binding fragment thereof, according to the present invention, a nucleic acid molecule (or the plurality of nucleic acid molecules) according to the present invention, a vector (or the plurality of vectors) according to the present invention, a cell according to the present invention, a (pharmaceutical) composition according to the present invention or a kit according to the present invention.

Moreover, the present invention also provides the use of an antibody according to the present invention, or an antigen-binding fragment thereof, a nucleic acid molecule (or the plurality of nucleic acid molecules) according to the present invention, a vector (or the plurality of vectors) according to the present invention, a cell according to the present invention, a pharmaceutical composition according to the present invention or a kit according to the present invention in the manufacture of a medicament for prophylaxis, treatment or attenuation of a birch pollen allergy and/or related tree pollen allergy, which preferably selected from at least one of hazel pollen allergy, black alder pollen allergy, beech pollen allergy, oak tree pollen allergy, chestnut pollen allergy, hop-hornbeam pollen allergy and hornbeam pollen allergy, and/or a related food allergy, which is preferably selected from at least one of apple allergy, hazelnut allergy and walnut allergy, or of a symptom of a birch pollen allergy and/or related (tree pollen and/or food) allergy, as described herein, such as an anaphylactic reaction due to a birch pollen allergy and/or related (tree pollen and/or food) allergy as described herein.

As used herein, the terms “treat” or “treatment” include therapeutic treatment and prophylactic or preventative measures. Prophylaxis of a birch pollen allergy and/or related (tree pollen and/or food) allergy refers in particular to prophylactic settings, wherein the subject was either not diagnosed with a birch pollen allergy and/or related (tree pollen and/or food) allergy (either no diagnosis was performed or diagnosis results were negative) and/or the subject does not show symptoms of a birch pollen allergy and/or related (tree pollen and/or food) allergy. Prophylaxis of a symptom of a birch pollen allergy and/or related (tree pollen and/or food) allergy, such as an anaphylactic reaction due to a birch pollen allergy and/or related (tree pollen and/or food) allergy, refers in particular to prophylactic settings, wherein the subject is not currently experiencing a symptom of a birch pollen allergy and/or related (tree pollen and/or food) allergy, such as an anaphylactic reaction due to a birch pollen allergy and/or related (tree pollen and/or food) allergy, but may possibly expect to experience a symptom of a birch pollen allergy and/or related (tree pollen and/or food) allergy, such as an anaphylactic reaction due to a birch pollen allergy and/or related (tree pollen and/or food) allergy, in the (near) future, e.g. due to the exposure to substances (e.g., via inhalation, ingestion) of unknown components or known to contain birch pollen and/or related tree pollen and/or related food allergens. In therapeutic settings, in contrast, the subject is typically diagnosed with a birch pollen allergy and/or related (tree pollen and/or food) allergy and/or shows symptoms of a birch pollen allergy and/or related (tree pollen and/or food) allergy, as described herein. Of note, the terms “treatment” and “therapy”/“therapeutic” include (complete) cure as well as attenuation/reduction of a birch pollen allergy (and/or related (tree pollen and/or food) allergy, as described herein) and/or related symptoms.

In general, the object of the “treatment” may be to decrease, ameliorate, inhibit, prevent or slow down (lessen or delay) an undesired physiological change or disorder, such as the birch pollen allergy and/or related (tree pollen and/or food) allergy or a symptom of a birch pollen allergy and/or related (tree pollen and/or food) allergy, such as an anaphylactic reaction due to a birch pollen allergy and/or related (tree pollen and/or food) allergy. Beneficial or desired clinical results of a treatment include, but are not limited to, alleviation of symptoms, diminishment of the extent of a disease, stabilized (i.e., not worsening) state of disease, delay or slowing of disease progression, amelioration or palliation of the disease state, and remission (whether partial or total), whether detectable or undetectable. “Treatment” can also mean prolonging survival, e.g. as compared to expected survival if not receiving treatment. Those in need of treatment include those already with the condition or disorder as well as those prone to have the condition or disorder or those in which the manifestation of the condition or disorder or the risk thereof is to be decreased, delayed or prevented.

In some embodiments the subject may be a human. The human may be selected from the group of a human suffering from birch pollen allergy and/or related (tree pollen and/or food) allergy, as described herein, an allergen-sensitized human without clinical relevant allergy, a human suffering from birch pollen allergy and/or related (tree pollen and/or food) allergy, as described herein, that underwent immunotherapy, a human at risk of developing a birch pollen allergy and/or related (tree pollen and/or food) allergy and a human of unknown clinical history for birch pollen allergy and/or related (tree pollen and/or food) allergy.

Symptoms of birch pollen allergy and/or related (tree pollen and/or food) allergy, as described herein, may include one or more of skin rash, itching skin, itching or tingling sensation in or around the mouth or throat, headache, sneezing, swelling, nausea, diarrhoea or anaphylaxis. Whether or not a subject suffers from birch pollen allergy and/or a related (tree pollen and/or food) allergy may be determined by correlation of allergic symptoms to contact with (e.g. inhalation, ingestion) birch pollen allergens or related allergens, as described herein. Additionally or alternatively, an allergen skin test and/or a blood test (for birch pollen-allergy IgE) may be performed.

One way of checking efficacy of therapeutic treatment involves monitoring disease symptoms after administration of the antibody or of the composition. Treatment can be a single dose schedule or a multiple dose schedule. In some embodiments, an antibody, antibody fragment, nucleic acid, vector, cell, composition or kit, as described herein, may be administered to a subject in need of such treatment. Such a subject includes, but is not limited to, one who is particularly at risk of, or susceptible to, a birch pollen and/or related (tree pollen and/or food) allergy or a symptom of a birch pollen and/or related (tree pollen and/or food) allergy.

The antibody according to the present invention, or an antigen-binding fragment thereof, the nucleic acid molecule (or the plurality of nucleic acid molecules) according to the present invention, the vector (or the plurality of vectors) according to the present invention, the cell according to the present invention, the pharmaceutical composition according to the present invention or the kit according to the present invention may be administered by any route of administration including, but not limited to, oral, intravenous, intramuscular, intra-arterial, intraperitoneal, transdermal, transcutaneous, topical, subcutaneous, intranasal, enteral, sublingual or rectal routes. In addition, any gene therapy approaches may be used, e.g. the antibody according to the present invention, or an antigen-binding fragment thereof, may be administered as nucleic acid or vector encoding said antibody, e.g. using viral or non-viral vectors as described above. In some embodiments, the antibody according to the present invention, or an antigen-binding fragment thereof, the nucleic acid molecule (or the plurality of nucleic acid molecules) according to the present invention, the vector (or the plurality of vectors) according to the present invention, the cell according to the present invention or the pharmaceutical composition according to the present invention may be administered systemically, for example by intravenous or subcutaneous administration.

### Combination Treatments

In some embodiments, co-administration or sequential administration of (i) an antibody according to the present invention, or an antigen-binding fragment thereof, a nucleic acid molecule (or the plurality of nucleic acid molecules) according to the present invention, a vector (or the plurality of vectors) according to the present invention, a cell according to the present invention or the pharmaceutical composition according to the present invention and (ii) a co-agent may be desirable. In some instances, the co-agent may be comprised in the pharmaceutical composition.

Preferably, the above-described medical uses comprise (combined) administration of at least two or three distinct antibodies or antigen-binding fragments thereof, which bind to distinct non-overlapping epitopes of Bet v 1, and optionally bind to distinct non-overlapping epitopes of at least one Bet v 1 homologous protein; or nucleic acid(s) or vector(s) encoding the distinct antibodies; or composition(s) comprising the distinct antibodies.

While the following description refers to administration of distinct antibodies, or antigen-binding fragments thereof, it is understood that the administration may likewise refer to (distinct) nucleic acid(s) encoding said antibodies, or antigen-binding fragments; or (distinct) composition(s) comprising said antibodies, or antigen-binding fragments, or said nucleic acid(s).

For combination, the distinct antibodies may be administered in the same or in different compositions. The distinct antibodies may be administered via the same or via distinct routes of administration. As used herein, “combined administration” means that the distinct antibodies are administered in a combined treatment scheme, i.e. a treatment scheme including the administration of the distinct antibodies (in contrast to a treatment scheme relating to a single antibody only, followed by a treatment scheme with another (single) antibody). Thereby, the antibodies may be administered simultaneously (e.g., during the same treatment session) or sequentially, e.g. on different days.

Preferably, the above-described medical uses or treatments comprise (combined) administration of at least two distinct antibodies, or antigen-binding fragments thereof, binding to distinct, non-overlapping epitopes of Bet v 1 and/or a Bet v 1 homologous protein. More preferably, the above-described medical uses comprise (combined) administration of at least three distinct antibodies, or antigen-binding fragments thereof, binding to distinct, non-overlapping epitopes of Bet v 1 and/or a Bet v 1 homologous protein. To this end, the antibodies administered in combination may be selected from the antibodies 4E1, 25-3D3, 17B10, 6C9, 3F1 and 13A2 or variants thereof, as described above, e.g. in Tables 1 and 2. As shown in the appended examples, 4E1, 25-3D3 and 17B10/6C9/3F1/13A2 bind to distinct, non-overlapping epitopes of Bet v 1.

Accordingly, the medical use or treatment may comprise (combined) administration of at least two of the following:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above;
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above; and/or an antibody, or an antigen-binding fragment thereof, selected from any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B10, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 13A2, or sequence variants thereof, as described above.

In some embodiments, the medical use or treatment may comprise (combined) administration of (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above; and
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above.

In some embodiments, the medical use or treatment may comprise (combined) administration of (exactly) two distinct antibodies, namely:
(i) an antibody, or an antigen-binding fragment thereof, comprising (a) the CDRH1, CDRH2, CDRH3, CDRL1, CDRL2 and CDRL3 sequences and/or (b) the VH and VL sequences of 4E1, or sequence variants thereof, as described above; and an antibody, or an antigen-binding fragment thereof, selected from any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B10, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 13A2, or sequence variants thereof, as described above.

In some embodiments, the medical use or treatment may comprise (combined) administration of (exactly) two distinct antibodies, namely:
(ii) an antibody, or an antigen-binding fragment thereof, comprising (a) the CDRH1, CDRH2, CDRH3, CDRL1, CDRL2 and CDRL3 sequences and/or (b) the VH and VL sequences of 25-3D3, or sequence variants thereof, as described above; and an antibody, or an antigen-binding fragment thereof, selected from any one of (iii) to (vi):
(iii) an antibody, or an antigen-binding fragment thereof, comprising (a) the CDRH1, CDRH2, CDRH3, CDRL1, CDRL2 and CDRL3 sequences and/or (b) the VH and VL sequences of 17B110, or sequence variants thereof, as described above;
(iv) an antibody, or an antigen-binding fragment thereof, comprising (a) the CDRH1, CDRH2, CDRH3, CDRL1, CDRL2 and CDRL3 sequences and/or (b) the VH and VL sequences of 6C9, or sequence variants thereof, as described above;
(v) an antibody, or an antigen-binding fragment thereof, comprising (a) the CDRH1, CDRH2, CDRH3, CDRL1, CDRL2 and CDRL3 sequences and/or (b) the VH and VL sequences of 3F1, or sequence variants thereof, as described above;
(vi) an antibody, or an antigen-binding fragment thereof, comprising (a) the CDRH1, CDRH2, CDRH3, CDRL1, CDRL2 and CDRL3 sequences and/or (b) the VH and VL sequences of 13A2, or sequence variants thereof, as described above.

Preferably, the medical use or treatment comprises (combined) administration of at least three antibodies, or antigen-binding fragments, binding to distinct, non-overlapping epitopes of Bet v 1, and optionally binding to at least one Bet v 1 homologous protein, wherein the at least three distinct antibodies, or antigen-binding fragments, are preferably (i), (ii) and any one selected from (iii)-(vi), as defined above.

The present invention also provides at least three distinct antibodies, or antigen-binding fragments thereof, binding to distinct, non-overlapping epitopes of Bet v 1 and optionally binding to at least one Bet v 1 homologous protein for use as a medicament, preferably for prophylaxis or treatment of a birch pollen allergy or related (tree pollen and/or food) allergy or a symptom of a birch pollen allergy or related (tree pollen and/or food) allergy, comprising (combined) administration of at least two of the following:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14; and an antibody, or an antigen-binding fragment thereof, selected from any one of (iii) to (vi):
(iii) an antibody, or an antigen-binding fragment thereof, comprising a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antibody, or an antigen-binding fragment thereof, comprising a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at least 70% identity to SEQ ID NO: 70, and a CDRL3 having at least 70% identity to SEQ ID NO: 71;
(v) an antibody, or an antigen-binding fragment thereof, comprising a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79;
(vi) an antibody, or an antigen-binding fragment thereof, comprising a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87.

Preferably, the medical use/treatment as described above comprises (combined) administration of at least two of the following:
(i) an antibody, or an antigen-binding fragment thereof, comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14; and an antibody, or an antigen-binding fragment thereof, selected from any one of (iii) to (vi):
(iii) an antibody, or an antigen-binding fragment thereof, comprising a CDRH1 according to SEQ ID NO: 17, a CDRH2 according to SEQ ID NO: 18, a CDRH3 according to SEQ ID NO: 19, a CDRL1 according to SEQ ID NO: 20, a CDRL2 according to SEQ ID NO: 21, and a CDRL3 according to SEQ ID NO: 22;
(iv) an antibody, or an antigen-binding fragment thereof, comprising a CDRH1 according to SEQ ID NO: 66, a CDRH2 according to SEQ ID NO: 67, a CDRH3 according to SEQ ID NO: 68, a CDRL1 according to SEQ ID NO: 69, a CDRL2 according to SEQ ID NO: 70, and a CDRL3 according to SEQ ID NO: 71;
(v) an antibody, or an antigen-binding fragment thereof, comprising a CDRH1 according to SEQ ID NO: 74, a CDRH2 according to SEQ ID NO: 75, a CDRH3 according to SEQ ID NO: 76, a CDRL1 according to SEQ ID NO: 77, a CDRL2 according to SEQ ID NO: 78, and a CDRL3 according to SEQ ID NO: 79;
(vi) an antibody, or an antigen-binding fragment thereof, comprising a CDRH1 according to SEQ ID NO: 82, a CDRH2 according to SEQ ID NO: 83, a CDRH3 according to SEQ ID NO: 84, a CDRL1 according to SEQ ID NO: 85, a CDRL2 according to SEQ ID NO:86, and a CDRL3 according to SEQ ID NO: 87.

More preferably, the medical use/treatment as described above comprises (combined) administration of at least two of the following:
(i) an antibody, or an antigen-binding fragment thereof, comprising a VH having at least 70% identity to SEQ ID NO: 7; and a VL having at least 70% identity to SEQ ID NO: 8;
(ii) an antibody, or an antigen-binding fragment thereof, comprising a VH having at least 70% identity to SEQ ID NO: 15; and a VL having at least 70% identity to SEQ ID NO: 16; and an antibody, or an antigen-binding fragment thereof, selected from any one of (iii) to (vi):
(iii) an antibody, or an antigen-binding fragment thereof, comprising a VH having at least 70% identity to SEQ ID NO: 23; and a VL having at least 70% identity to SEQ ID NO: 24;
(iv) an antibody, or an antigen-binding fragment thereof, comprising a VH having at least 70% identity to SEQ ID NO: 72; and a VL having at least 70% identity to SEQ ID NO: 73;
(v) an antibody, or an antigen-binding fragment thereof, comprising a VH having at least 70% identity to SEQ ID NO: 80; and a VL having at least 70% identity to SEQ ID NO: 81;
(vi) an antibody, or an antigen-binding fragment thereof, comprising a VH having at least 70% identity to SEQ ID NO: 88; and a VL having at least 70% identity to SEQ ID NO: 89.

Even more preferably, the medical use/treatment as described above comprises (combined) administration of at least two of the following:
(i) an antibody, or an antigen-binding fragment thereof, comprising a VH according to SEQ ID NO: 7; and a VL according to SEQ ID NO: 8;
(ii) an antibody, or an antigen-binding fragment thereof, comprising a VH according to SEQ ID NO: 15; and a VL according to SEQ ID NO: 16; and an antibody, or an antigen-binding fragment thereof, selected from any one of (iii) to (vi):
(iii) an antibody, or an antigen-binding fragment thereof, comprising a VH according to SEQ ID NO: 23; and a VL according to SEQ ID NO: 24;
(iv) an antibody, or an antigen-binding fragment thereof, comprising a VH according to SEQ ID NO: 72; and a VL according to SEQ ID NO: 73;
(v) an antibody, or an antigen-binding fragment thereof, comprising a VH according to SEQ ID NO: 80; and a VL according to SEQ ID NO: 81;
(vi) an antibody, or an antigen-binding fragment thereof, comprising a VH according to SEQ ID NO: 88; and a VL according to SEQ ID NO: 89.

Thus, in a preferred embodiment, the medical use/treatment as described above comprises (combined) administration of:
(i) an antibody, or an antigen-binding fragment thereof, comprising:
   a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
   in particular a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
   in some embodiments a VH having at least 70% identity to SEQ ID NO: 7; and a VL having at least 70% identity to SEQ ID NO: 8; wherein the CDRH1 according to SEQ ID NO: 1, CDRH2 according to SEQ ID NO: 2, CDRH3 according to SEQ ID NO: 3, CDRL1 according to SEQ ID NO: 4, CDRL2 according to SEQ ID NO: 5, and CDRL3 according to SEQ ID NO: 6 are preferably maintained;
   such as a VH according to SEQ ID NO: 7; and a VL according to SEQ ID NO: 8; and
(ii) an antibody, or an antigen-binding fragment thereof, comprising:
   a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14;
   in particular a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14;
   in some embodiments a VH having at least 70% identity to SEQ ID NO: 15; and a VL having at least 70% identity to SEQ ID NO: 16; wherein the CDRH1 according to SEQ ID NO: 9, CDRH2 according to SEQ ID NO: 10, CDRH3 according to SEQ ID NO: 11, CDRL1 according to SEQ ID NO: 12, CDRL2 according to SEQ ID NO: 13, and CDRL3 according to SEQ ID NO: 14 are preferably maintained;
   such as a VH according to SEQ ID NO: 15; and a VL according to SEQ ID NO: 16; and/or an antibody, or an antigen-binding fragment thereof, selected from any one of (iii) to (vi):
(iii) an antibody, or an antigen-binding fragment thereof, comprising:
   a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22;
   in particular a CDRH1 according to SEQ ID NO: 17, a CDRH2 according to SEQ ID NO: 18, a CDRH3 according to SEQ ID NO: 19, a CDRL1 according to SEQ ID NO: 20, a CDRL2 according to SEQ ID NO: 21, and a CDRL3 according to SEQ ID NO: 22;
   in some embodiments a VH having at least 70% identity to SEQ ID NO: 23; and a VL having at least 70% identity to SEQ ID NO: 24; wherein the CDRH1 according to SEQ ID NO: 17, CDRH2 according to SEQ ID NO: 18, CDRH3 according to SEQ ID NO: 19, CDRL1 according to SEQ ID NO: 20, CDRL2 according to SEQ ID NO: 21, and CDRL3 according to SEQ ID NO: 22 are preferably maintained;
   such as a VH according to SEQ ID NO: 23; and a VL according to SEQ ID NO: 24;
(iv) an antibody, or an antigen-binding fragment thereof, comprising:
   a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at least 70% identity to SEQ ID NO: 70, and a CDRL3 having at least 70% identity to SEQ ID NO: 71;
   in particular a CDRH1 according to SEQ ID NO: 66, a CDRH2 according to SEQ ID NO: 67, a CDRH3 according to SEQ ID NO: 68, a CDRL1 according to SEQ ID NO: 69, a CDRL2 according to SEQ ID NO: 70, and a CDRL3 according to SEQ ID NO: 71;
   in some embodiments a VH having at least 70% identity to SEQ ID NO: 72; and a VL having at least 70% identity to SEQ ID NO: 73; wherein the CDRH1 according SEQ ID NO: 66, CDRH2 according to SEQ ID NO: 67, CDRH3 according to SEQ ID NO: 68, CDRL1 according to SEQ ID NO: 69, CDRL2 according to SEQ ID NO: 70, and CDRL3 according to SEQ ID NO: 71 are preferably maintained;
   such as a VH according to SEQ ID NO: 72; and a VL according to SEQ ID NO: 73;
(v) an antibody, or an antigen-binding fragment thereof, comprising:
   a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79;
   in particular a CDRH1 according to SEQ ID NO: 74, a CDRH2 according to SEQ ID NO: 75, a CDRH3 according to SEQ ID NO: 76, a CDRL1 according to SEQ ID NO: 77, a CDRL2 according to SEQ ID NO: 78, and a CDRL3 according to SEQ ID NO: 79;
   in some embodiments a VH having at least 70% identity to SEQ ID NO: 80; and a VL having at least 70% identity to SEQ ID NO: 81; wherein the CDRH1 according SEQ ID NO: 74, CDRH2 according to SEQ ID NO: 75, CDRH3 according to SEQ ID NO: 76, CDRL1 according to SEQ ID NO: 77, CDRL2 according to SEQ ID NO: 78, and CDRL3 according to SEQ ID NO: 79 are preferably maintained;
   such as a VH according to SEQ ID NO: 80; and a VL according to SEQ ID NO: 81;
(vi) an antibody, or an antigen-binding fragment thereof, comprising:
   a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87;
   in particular a CDRH1 according to SEQ ID NO: 82, a CDRH2 according to SEQ ID NO: 83, a CDRH3 according to SEQ ID NO: 84, a CDRL1 according to SEQ ID NO: 85, a CDRL2 according to SEQ ID NO: 86, and a CDRL3 according to SEQ ID NO: 87;
   in some embodiments a VH having at least 70% identity to SEQ ID NO: 88; and a VL having at least 70% identity to SEQ ID NO: 89; wherein the CDRH1 according SEQ ID NO: 82, CDRH2 according to SEQ ID NO: 83, CDRH3 according to SEQ ID NO: 84, CDRL1 according to SEQ ID NO: 85, CDRL2 according to SEQ ID NO: 86, and CDRL3 according to SEQ ID NO: 87 are preferably maintained;
   such as a VH according to SEQ ID NO: 88; and a VL according to SEQ ID NO: 89.

Preferably, the medical use/treatment as described above comprises (combined) administration of antibodies, or antigen-binding fragments thereof, binding to three distinct, non-overlapping epitopes on Bet v 1 and optionally binding to three distinct, non-overlapping epitopes on at least one Bet v 1 homologous protein. Accordingly, in a preferred embodiment, the medical use/treatment comprises (combined) administration of three distinct antibodies, or antigen-binding fragments, wherein a first antibody, or antigen-binding fragments thereof, is according to (i), a second antibody, or antigen-binding fragments thereof, is according to (ii), and a third antibody, or antigen-binding fragments thereof, is selected from an antibody, or antigen-binding fragments thereof, according to any one of (iii) to (vi) above.

As described above, the medical use/treatment of the invention preferably comprises (combined) administration of antibodies binding to at least two, preferably three, distinct non-overlapping epitopes of Bet v 1, and optionally binding to at least two, preferably three, distinct non-overlapping epitopes of at least one Bet v 1 homologous protein. Antibodies, or antigen-binding fragments thereof, which are administered (in combination), may be selected from the antibodies 4E1, 25-3D3, 17B10, 6C9, 3F1 and 13A2 or variants thereof, as described above. Preferably, two or three monospecific antibodies as described herein, may be administered (in combination). In some embodiments, multispecific antibodies, in particular as described herein, may (also) be administered.

Alternatively, or additionally, at least one additional agent useful for treating a birch pollen allergy and/or related (tree pollen and/or food) allergy may be administered (in combination) with the antibodies, or antigen-binding fragments, as described herein. The additional agent useful for treating a birch pollen allergy or related (tree pollen and/or food) allergy may be selected from the group comprising: a β-adrenergic agonist (e.g. epinephrine), antihistamine, a corticosteroid, an anti-IgE antibody, an anti-IgE antibody binding fragment, a peptide vaccine and further antibodies capable of binding to a birch pollen allergen and/or related (tree pollen and/or food) allergen. In some embodiments, the additional agent is a β-adrenergic agonist, such as epinephrine.

In some embodiments, a birch pollen allergen and/or related (tree pollen and/or food) allergen may be administered (in combination) with the antibodies, or antigen-binding fragments, as described herein. The birch pollen allergen and/or related allergen may be a Bet v 1 birch pollen allergen, and/or a related tree pollen allergen, which is preferably selected from at least one of Cor a 1 hazel pollen allergen, Ain g 1 black alder pollen allergen, Fag s 1 beech pollen allergen, Que a 1 oak tree pollen allergen, Cas s 1 chestnut pollen allergen, Ost c 1 hop-hornbeam pollen allergen, Car b1 hornbeam pollen allergen, and/or a related food allergen, which is preferably selected from at least one of Mal d 1 apple allergen, Cor a 1 hazelnut allergen and Jug r 5 walnut allergen. The birch pollen allergen and/or related (tree pollen and/or food) allergen may be of natural origin, recombinantly expressed or may be a synthetic peptide.

In some embodiments, the anti-bet v 1 allergen antibodies, or antigen-binding fragments thereof, as described herein, may be pre-incubated with the birch pollen allergen or related (tree pollen or food) allergen and may be administered as a mixture to a subject.

Combining a birch pollen allergen or related (tree pollen or food) allergen, for example selected from the group consisting of Bet v 1, Cor a 1, Ain g 1, Fag s 1, Que a 1, Cas s 1, Ost c 1, Car b1, Mal d 1, and Jug r 5 or a mixture thereof, with the antibodies of the invention increases the safety of administering the allergen (e.g. in desensitization). Furthermore, without being bound to any theory, the present inventors assume that the combination of antibodies with the respective allergen (targeted by the antibodies), i.e. the combination of passive (antibodies) and active (allergen) immunization, has a synergistic effect.

### Further Uses

Antibodies and fragments thereof as described herein may also be used for the (in-vitro) diagnosis of a birch pollen allergy, and/or a related tree pollen allergy, which is preferably selected from at least one of hazel pollen allergy, black alder pollen allergy, beech pollen allergy, oak tree pollen allergy, chestnut pollen allergy, hop-hornbeam pollen allergy and hornbeam pollen allergy, and/or a related food allergy, which is preferably selected from at least one of apple allergy, hazelnut allergy and walnut allergy.

Methods of diagnosis may include contacting an antibody with a sample. Such samples may be isolated from a subject, for example an isolated blood sample, such as whole blood, plasma or serum. The methods of diagnosis may also include the detection of an antigen/antibody complex, in particular following the contacting of an antibody with a sample. Furthermore, it may be tested, whether the sample contains antibodies competing with the antibodies as described herein, e.g. for allergen binding. This is typically performed in vitro, i.e. without any contact to the human or animal body. Examples of analytical methods are well-known to the person skilled in the art and include immunoassays such as flow cytometry, dot or slot blots, Western blots, ELISA (enzyme-linked immunosorbent assay), e.g. for cross-competition, immunohistochemistry and immunoprecipitation followed by SDS-PAGE immunocytochemistry. Accordingly, the diagnosis may be performed in vitro, for example by using an isolated sample as described above (and an in vitro analysis step as described above).

The present invention also provides a method of detecting whether a sample comprises a birch pollen allergen, in particular Bet v 1, or a Bet v 1 homologous allergen, such as Cor a 1 hazel pollen allergen, Aln g 1 black alder pollen allergen, Fag s 1 beech pollen allergen, Que a 1 oak tree pollen allergen, Cas s 1 chestnut pollen allergen, Ost c 1 hop-hornbeam pollen allergen, Car b1 hornbeam pollen allergen, Mal d 1 apple allergen, and Jug r 5 walnut allergen. Such a method may comprise the following steps:
a. contacting the sample with the antibody described herein under conditions permissive to produce an antibody/antigen complex; and
b. detecting the presence of the antibody/antigen complex.

Thereby, the presence of detectable antibody/antigen complex may be indicative that the sample may contain a birch pollen allergen, in particular Bet v 1, and/or a Bet v 1 homologous allergen, such as Cor a 1 hazel pollen allergen, Ain g 1 black alder pollen allergen, Fag s 1 beech pollen allergen, Que a 1 oak tree pollen allergen, Cas s 1 chestnut pollen allergen, Ost c 1 hop-hornbeam pollen allergen, Car b1 hornbeam pollen allergen, Mal d 1 apple allergen, and Jug r 5 walnut allergen.

In some embodiments, in step (b) the amount of the antigen/antibody complex may be determined (e.g., measured) in the test sample. Thereafter, said amount may be compared to a control.

Accordingly, the antibody of the present invention, or an antigen-binding fragment thereof, may be used in an (in vitro) method for detecting a birch pollen allergen, in particular Bet v 1, and/or a Bet v 1 homologous allergen, as described above. Likewise, the antibody of the present invention, or an antigen-binding fragment thereof, may be used in an (in vitro) method for binding a birch pollen allergen, in particular Bet v 1, and/or a Bet v 1 homologous allergen, as described above. For detecting a birch pollen allergen, in particular Bet v 1, and/or a Bet v 1 homologous allergen, the antibody may be brought in contact with a (isolated) sample (i.e., a sample to be tested for the presence of the antigen). By the specific binding of the antibody to its antigen (a birch pollen allergen, in particular Bet v 1, and/or a Bet v 1 homologous allergen), an antibody/antigen complex is formed, which can be easily detected by methods known in the art.

Such a detection method may be used for testing samples (e.g., production/manufacture), such as food, cosmetics or medication samples. Accordingly, antibodies, antibody fragment, or variants thereof, as described in the present invention may also be used in a non-therapeutic/non-diagnostic context, e.g. in development or manufacture of various products. Furthermore, the detection method may be used for testing vaccine samples (e.g. for use in desensitization), whether they contain the major birch pollen allergen Bet v 1, and/or a Bet v 1 homologous allergen. This may be useful in the development and/or manufacture of such vaccines. Accordingly, the present invention therefore also provides the use of the antibodies as described herein, or an antigen-binding fragment thereof, for testing immunogenic compositions/vaccines, in particular of an immunogenic composition/vaccine comprising a birch pollen allergen, in particular Bet v 1, and/or a Bet v 1 homologous allergen. To this end, the antibody may be brought in contact with the immunogenic composition/vaccine, e.g. to test for antibody/antigen complexes. Accordingly, the present invention also provides a method for testing immunogenic compositions (vaccines) based on a birch pollen allergen, in particular Bet v 1, and/or a Bet v 1 homologous allergen, wherein the immunogenic composition/vaccine is contacted with the antibody, or an antigen-binding fragment thereof, and, optionally, the presence of antibody/antigen complexes is determined. Furthermore, the present invention also encompasses the use of the antibody of the present invention, or an antigen-binding fragment thereof, for monitoring the quality of immunogenic compositions/vaccines based on a birch pollen allergen, in particular Bet v 1, and/or a Bet v 1 homologous allergen, by checking whether the immunogenic composition/vaccine contains the desired antigen, e.g. Bet v 1, Cor a 1, Aln g 1, Fag s 1, Que a 1, Cas s 1, Ost c 1, Car b1, Mal d1, jug d 5 etc.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows for Example 1 the BLI sensogram for MY010 antibodies 4E1, 25-3D3 and 17B10 binding to Bet v 1. Capture mAb1, as indicated, was immobilized and Bet v 1, mAb2 and mAb3, as indicated, were injected sequentially as indicated in the Figure.

FIGS. 2A-2G show for Example 2 the affinity of the inventive MY010 antibodies 4E1 (FIG. 2A), 25-3D3 (FIG. 2B), and 17B10 (FIG. 2C) on birch extracts and birch related extracts (hazel pollen extract, black alder extract, European beech extract, oak extract) compared to the affinity of comparative prior art antibodies REGN-5713 (FIG. 2D), REGN-5714 (FIG. 2E) and REGN-5715 (FIG. 2F), as measured by competition ELISA. FIG. 2G summarizes the affinity of the inventive and prior art antibodies in a heat map.

FIGS. 3A-3C show for Example 3 the affinity of the inventive MY010 antibodies 25-3D3, 17B110 and 4E1 to Bet v 1 and Bet v 1 homologous allergens (hazel Cor a 1, alder Aln g 1, beech Fag s 1, oak Que a 1, chestnut Cas s 1, hop hornbeam Ost c 1, and hornbeam Car b 1 (FIG. 3A), in comparison to the affinity of the prior art antibodies REGN-5713, REGN-5714 and REGN-5715 (FIG. 3B), as measured by binding ELISA. FIG. 3C summarizes the affinity of the inventive and prior art antibodies in a heat map.

FIGS. 4A-4G show for Example 4 the affinity of the inventive MY010 antibodies 4E1 (FIG. 4A), 25-3D3 (FIG. 4B), 17B10 (FIG. 4C) to food-related Bet v 1 homologous allergens (apple Mal d 1, hazelnut Cor a 1 and walnut Jug r 5) in comparison to the affinity of the prior art antibodies REGN-5713 (FIG. 4D), REGN-5714 (FIG. 4E) and REGN-5715 (FIG. 4F), as measured by soluble competition ELISA. FIG. 4G summarizes the affinity of the inventive and comparative antibodies in a heat map.

FIGS. 5A-5B show for Example 5 the suppression of mast cell responsiveness by “cocktails” containing one (4E1), two (4E1+25-3D3; 4E1+17B10) or three (4E1+17B10+25-3D3) of the inventive MY010 antibodies, compared to a “cocktail” containing the prior art antibodies REGN-5713, REGN-5714 and REGN-5715 (REGN-3). Bone marrow derived mast cells isolated from transgenic mice expressing the human FcERIa are sensitized with plasma pool from birch pollen allergic patients (n=6). Activation of mast cells is quantified in terms of % CD107a positive cells. Titrated antibody-Cocktails (80 nM-0 nM) are pre-incubated with fixed concentrations of extracts. Cells are sensitized with plasma pool overnight at 37° C. and then stimulated for 35 minutes at 37° C. with the pre-incubated extracts.

FIGS. 6A-6B show for Example 6 the suppression of mast cell responsiveness by the inventive “MY010-cocktail” containing three (4E1+17B10+25-3D3) of the inventive antibodies, compared to a “REGN-cocktail” containing the prior art antibodies REGN-5713, REGN-5714 and REGN-5715. Titrated allergen/extract is pre-incubated with fixed concentration of antibodies (80 nM).

FIG. 7 shows for Example 7 the prevention of birch extract induced (10 ng/ml Bet v 1) degranulation of basophils in patient whole blood samples by the inventive “MY010-Cocktail” (4E1+17B10+25-3D3) compared to the prior art REGN-Cocktail (REGN-5713+REGN-5714+REGN-5715), quantified by % CD63 expression. Bars indicate activation of basophils expressed as % CD63+ cells (mean±s.d, n=2). White bars: no addition of antibody. Data from 1 representative patient. Statistics done by One way anova followed by Dunnett's test comparing each column to 10 ng/ml birch extract.

FIG. 8 shows for Example 8 the efficacy of the “MY010-Cocktail” on birch related food allergens (apple Mal d 1, hazelnut Cor a 1, walnut Jug r 5, peach extract), as measured by mast cell activation test.

FIG. 9 shows for Example 9 competition of MY010 antibody complementation groups measured by cross-competition ELISA on Bet v 1.

FIG. 10 shows for Example 10 the BLI sensogram for MY010 antibodies 4E1, 25-3D3 and 3F1 binding to Bet v 1. Capture mAb1, as indicated, was immobilized, and Bet v 1, mAb2 and mAb3, as indicated, were injected sequentially as indicated in the Figure.

FIGS. 11A-11D show for Example 11 the affinity of the inventive MY010 antibodies 6C9 (FIG. 11A), 3F1 (FIG. 11B), and 13A2 (FIG. 11C) on birch extracts and birch related extracts (hazel pollen extract, black alder extract, American beech extract, European beech extract, oak extract), as measured by soluble competition ELISA. FIG. 11D summarizes the affinity of the inventive antibodies of the MY010-17B10 complementation group in a heat map.

FIGS. 12A-12B show for Example 12 the binding affinity of the inventive MY010 antibodies 6C9, 3F1 and 13A2 to Bet v 1 and Bet v 1 homologous allergens (alder Aln g 1, hazel Cor a 1.0101 and Cor a 1.0201, hornbeam rec Car b 11A and rec Carb b 1 isoform 2, hop hornbeam Ost c 1, oak Que a 1.0201 and Que a 1.0301, beech Fag s 1, and chestnut Cas s 1 (FIG. 12A), as measured by binding ELISA. FIG. 12B summarizes the affinity of the inventive and prior art antibodies in a heat map.

FIGS. 13A-13C show for Example 13 the affinity of the inventive MY010 antibodies 6C9 (FIG. 13A), 3F1 (FIG. 13B), and 13A2 (FIG. 13C) to food-related Bet v 1 homologous allergens (apple Mal d 1.0108, hazelnut Cor a 1.0401 and walnut Jug r 5.0101), as measured by soluble competition ELISA.

FIGS. 14A-14B show for Example 14 the suppression of mast cell responsiveness by different antibody “cocktails” as indicated, measured by a mast cell activation Test (MAT). FIG. 14A: plasma pool from 6 US patients stimulated with birch extract, black alder extract, hazel pollen extract, beech extract, white oak extract; FIG. 14B: plasma pool from US, UK and CH patients stimulated with white oak extracts.

FIGS. 15A-15D show for Example 15 efficacy of MY010-3 cocktails comprising inventive antibodies as indicated compared with prior art REGN-3 cocktail in a passive cutaneous anaphylaxis (PCA) model. FIG. 15A: experimental set-up; FIG. 15B: mice i.v. challenged with birch pollen extract (0.1 ug); FIG. 15C: mice i.v. challenged with hazel pollen extract (1 ug); FIG. 15D: mice i.v. challenged with oak pollen extract (1 ug).

FIGS. 16A-16B show for Example 16 identification of the binding sites of MY010 and prior art REGN antibodies to Bet v 1 by epitope mapping. FIG. 16A: Scanning mutagenesis of surface-exposed residues of Bet v 1. FIG. 16B: Schematically illustration of Bet v 1 epitopes targeted by MY010 and REGN antibodies.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Antibodies 4E1, 25-3D3 and 17B10 Bind to Distinct Non-Overlapping Epitopes on Bet v 1

### 1. Antibody Cloning

Human lymphocytes from peripheral blood of voluntary allergic patients were obtained and used as starting material for the cloning of fully human antibodies. Antibodies specific to major birch pollen-related allergens were isolated by molecular cloning of immunoglobulin genes obtained from single-cell sorted cells derived from short term oligoclonal cultures of activated memory B cells producing the antibodies of interest. Molecular cloning of human antibodies specific to birch pollen-related allergens was carried out according to Huangi, Doria-Rose N A, Longo N S, Laub L, Lin C L, Turk E, Kang B H, Migueles S A, Bailer R T, Mascola I R, Connors M. Isolation of human monoclonal antibodies from peripheral blood B cells. Nat Protoc. 2013 October; 8(10): 1907-15. doi: 10.1038/nprot.2013.n117, as described in detail in WO 2018/234383 A1.

CDR and VH/VL sequences (SEQ ID NOs) of fully human antibodies 4E1, 25-3D3 and 17B110 are shown in Table 4 below:

TABLE 4
SEQ ID NOs for CDR and VH/VL sequences
of antibodies 4E1, 25-3D3 and 17B10

| Antibody | CDRH1 | CDRH2 | CDRH3 | CDRL1 | CDRL2 | CDRL3 | VH | VL |
|---|---|---|---|---|---|---|---|---|
| 4E1 | 1 | 2 | 3 | 4 | 5 | 6 | 7 | 8 |
| 25-3D3 | 9 | 10 | 11 | 12 | 13 | 14 | 15 | 16 |
| 17B10 | 17 | 18 | 19 | 20 | 21 | 22 | 23 | 24 |

### 2. Antibody Expression and Purification

Efficient and scalable antibody expression was achieved through suspension HEK293 cells, employing a method adaptable to expression volumes ranging from 20 to 1000 mL. HEK293 cells were transfected with DNA encoding both human light and heavy chain IgG4 and polyethylenimine (PEI Max, Polysciences, USA). To attain optimal IgG production, the transfected cells were supplemented with Tryptone N1 (OrganoTechni, France) after 24 hours. Following a 5-7 day expression period, the supernatant, containing the recombinant antibodies, was harvested.

The purification process involved the utilization of either a HiTrap Protein A HP column (Cytiva, US). Protein A from Staphylococcus exhibits a strong affinity for the Fc region of IgG. The binding of protein A to IgG is achieved under neutral pH conditions, the antibody is then eluted using an acidic pH solution. The purified antibodies were subsequently dialyzed into PBS, concentrated, and stored at −80° C. for long-term storage.

### 3. Biolayer Interferometry

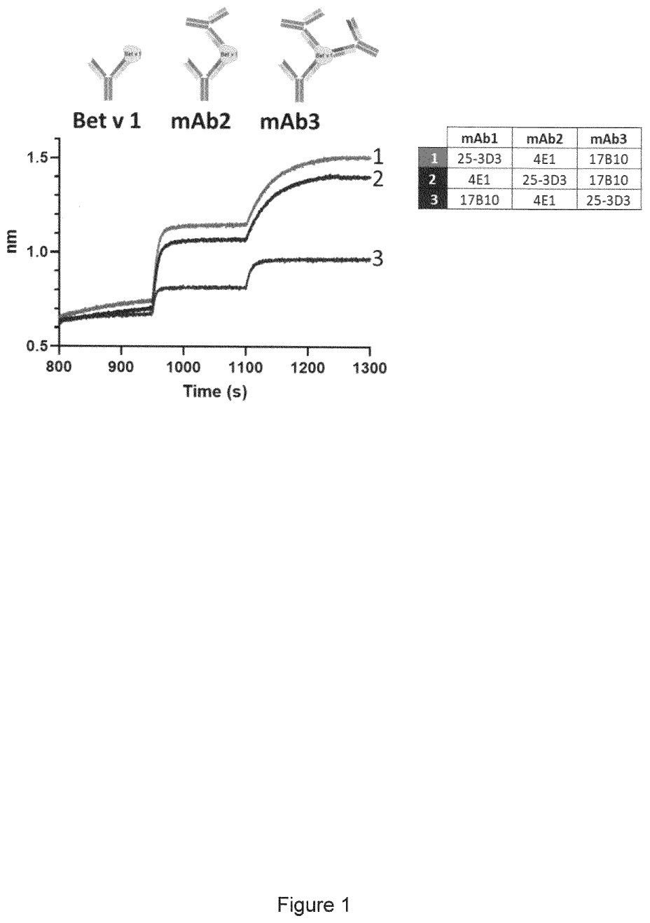

To determine whether antibodies 4E1, 25-3D3 and 17B10 can bind simultaneously to the epitopes on Bet v 1, biolayer interferometry (BLI; Forte Bio Octet RED96e; Running buffer: 1×PBS) was performed. IgG4 of the cocktail mAbs were used. First, mAbs1 were immobilized (5 ug/ml) on a human Fc binding BLI sensor surface, which were subsequently blocked with human Fc fragment (50 ug/ml). Then natural Bet v 1 (1.8 ug/ml) was applied followed by sequential injection of mAbs2 and mAbs3 (each 15 ug/ml), as indicated in the Table of FIG. 1.

The sensogram shown in FIG. 1 strongly suggests that all the IgGs can bind at the same time to one Bet v 1 molecule. Thus, the BLI data confirmed that all tested IgG antibodies 4E1, 25-3D3 and 17B10 can bind simultaneously and non-competitively to a single Bet v 1 molecule. Accordingly, these data demonstrate that 4E1, 25-3D3 and 17B10 antibodies bind to distinct, non-overlapping epitopes on Bet v 1.

### Example 2: Antibodies 4E1, 25-3D3 and 17B10 Bind to PR-10 Allergen Extracts with High Affinity and Show Broader Cross-Reactivity Compared to Prior Art Antibodies REGN-5713. REGN-5714, and REGN-5715

### 1. Preparation of Comparative Antibodies REGN-5713, REGN-5714 and REGN-5715

Amino acid and DNA sequences of REGN-5713, REGN-5714 and REGN-5715 were derived from patent U.S. Ser. No. 10/793,624B2. Respective V regions were cloned into expressions vectors containing either coding sequences for human IgG4 (S228P), for VH, or human kappa constant region, for VL sequences. Recombinant antibodies were transiently produced in HEK293 suspension cells and purified via Protein A affinity columns as described in Example 1, 2. Antibody Expression and Purification. Using Alanine scanning mutagenesis on Bet v 1, epitope specificity of REGN antibodies was confirmed (data not shown).

### 2. Soluble Competition ELISA

Cross reactivity of MY010 antibodies 4E1, 25-3D3 and 17B10, as well as of prior art antibodies REGN-5713, REGN-5714 and REGN-5715, to PR-10 related extracts (birch, hazel, black alder, European beech, oak) was checked by soluble competition ELISA.

Therefore plates were coated with streptavidin (1 ug/ml) overnight at 4° C. or 1 h at 37° C. followed by capturing of biotinylated natBet v 1 (1.4 nM) overnight at 4° C. Titrated human MY010-mAbs (4E1, 25-3D3, 17B10) (80 nM-0 nM) and REGN antibodies (REGN-5713, REGN-5714 and REGN-5715), respectively, were preincubated with dilution buffer (no extract condition=0 nM) or 14 nM of respective extract overnight at 4° C. or 1 h at RT. The pre-mixtures of MY010-mAbs/extract and REGN-mAbs/extract, respectively, were incubated on the ELISA plate for 15 minutes at room temperature. Binding of MY010 antibodies (4E1, 25-3D3, 17B10) and REGN antibodies (REGN-5713, REGN-5714, REGN-5715), respectively, to immobilized natBet v 1 was quantified by anti-hu-IgG-HRP.

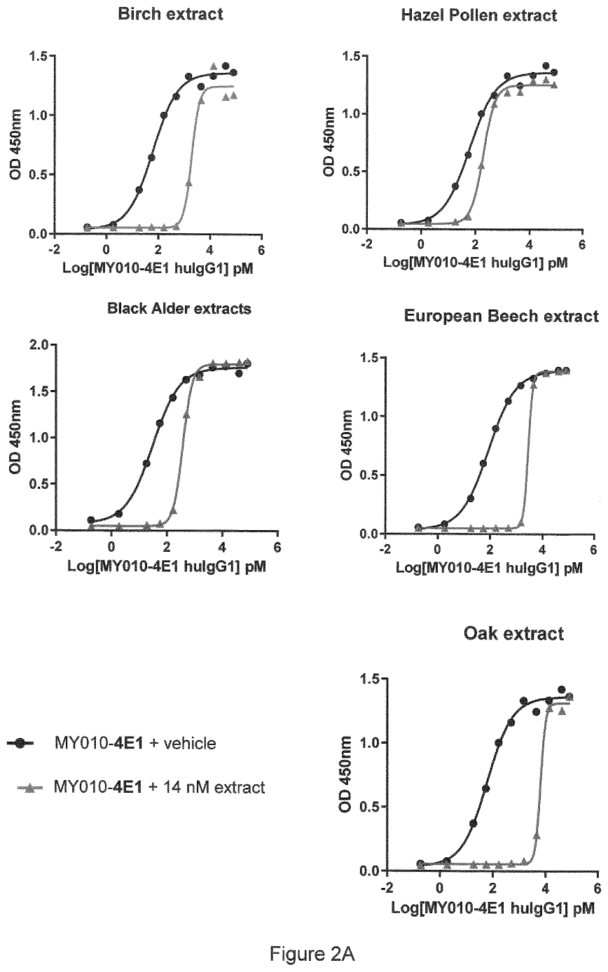

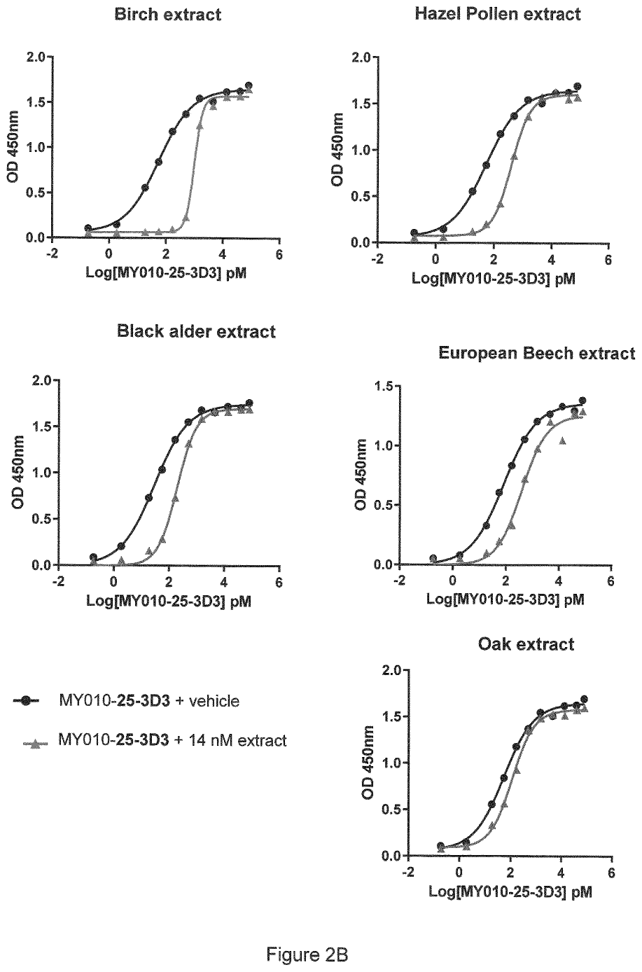

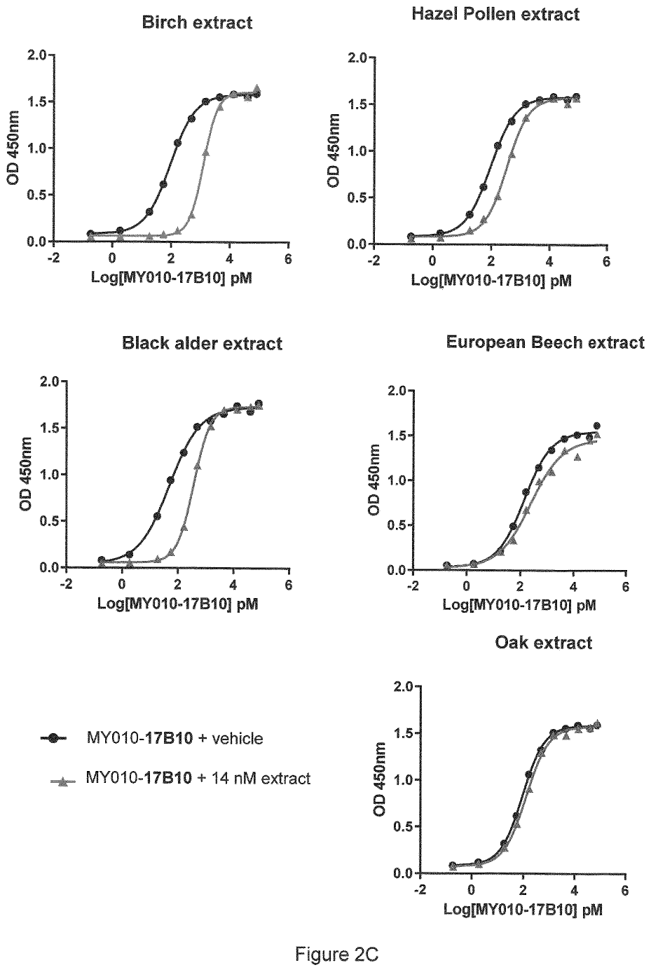

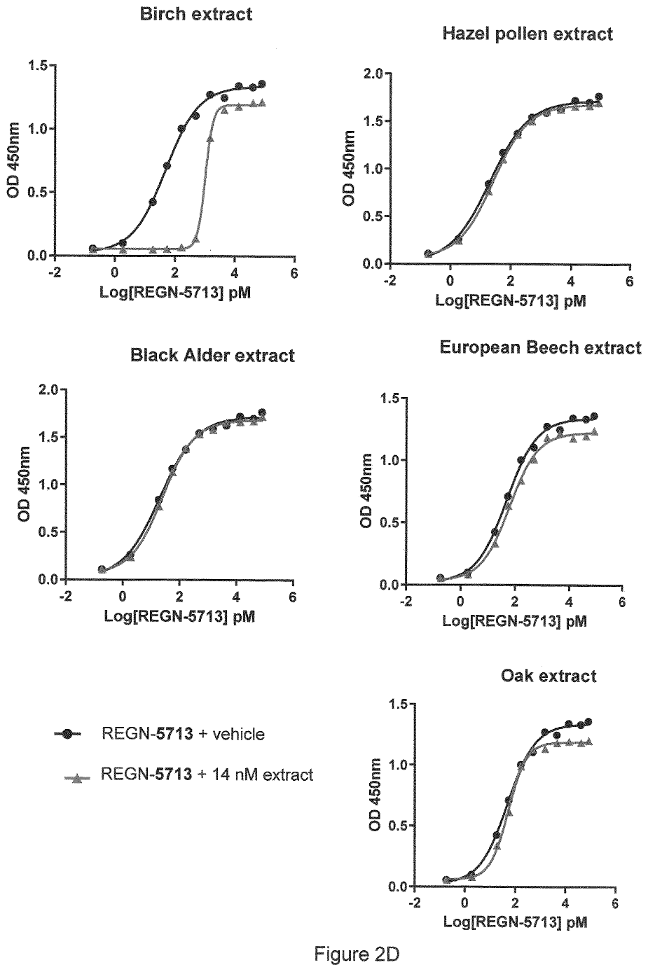

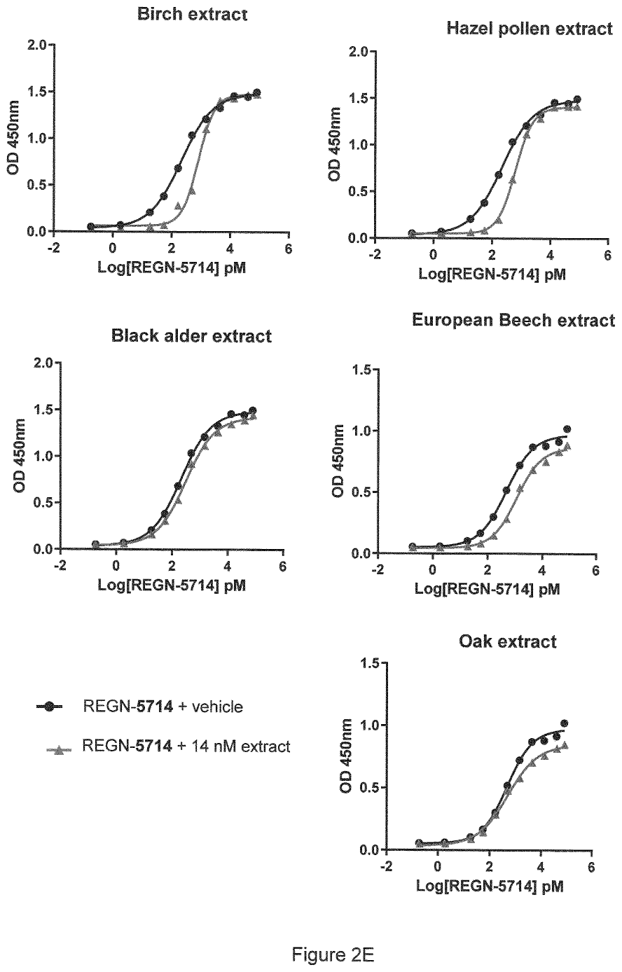

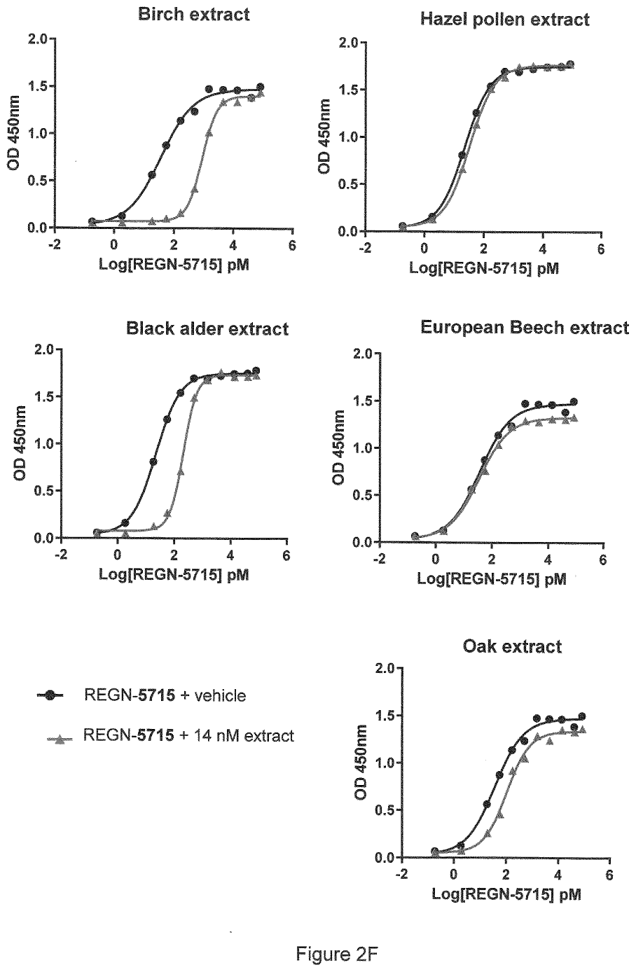

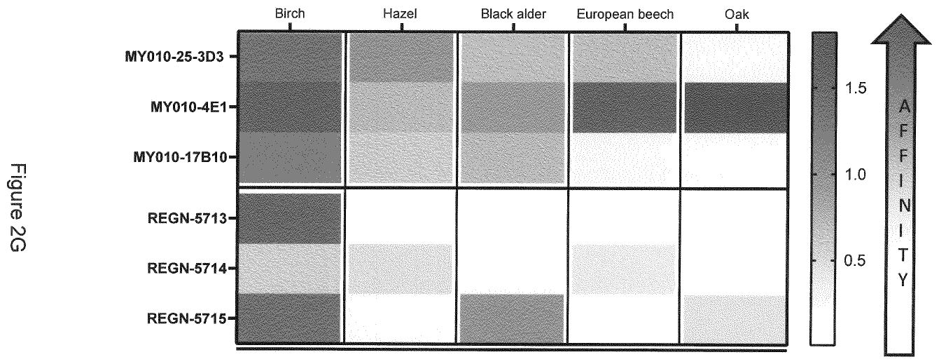

The results shown in FIG. 2A (mAb 4E1), FIG. 2B (mAb 25-3D3) and FIG. 2C (mAb 17B10) illustrate that MY010 antibodies 4E1, 25-3D3, 17B10 bind to PR-10 allergen extracts of birch, hazel, black alder, European beech and oak with high affinity and therefore show a broad cross-reactivity with respect to a wide spectrum of tree pollen allergens.

On the other hand, as illustrated in FIGS. 2D to 2F, the prior art mAbs REGN-5713 (FIG. 2D), REGN-5714 (FIG. 2E) and REGN-5715 (FIG. 2F) show a lower level of cross-reactivity with respect to birch-related PR-10 allergen extracts, in particular hazel, black alder, European beech and oak extracts. Thus, FIGS. 2A to 2F demonstrate the superior cross-reactivity of MY010 antibodies 4E1, 25-3D3 and 17B10 compared to prior art antibodies REGN-5713, REGN-5714, REGN-5715 with respect to a number of birch related tree pollen extracts.

The results are summarized in the heat map of FIG. 2G, where Δ Log EC50 is plotted: Δ Log EC₅₀=Log EC₅₀(14 nM)−Log EC₅₀(0 nM).

### Example 3: Antibodies 4E1, 25-3D3 and 17B110 are Highly Cross-Reactive to Recombinant PR-10 Proteins from Different Tree Pollen

In this experiment, a number of Bet v 1 related (PR-10) allergens were synthesized and used to define affinities of MY010 antibodies (4E1, 25-3D3 and 17B10) and REGN antibodies (REGN-5713, REGN-5714, REGN-5715) on birch related recombinant proteins:

Bet v 1.0101 (birch), Cor a 1.0101 and Cor a 1.0201 (hazel), Aln g 1.0101 (black alder), Fag s 1.0101 (beech), Que a 1.0201 (oak), Cas s 1.0101 (chestnut), Ost c 1.0101 (hop-hornbeam), Car b1.1A and Car b1.2 (hornbeam).

### 1. Expression of Recombinant PR-10 Proteins

PR-10 allergens were synthesized and cloned in the pET21 vector (Twist Biosciences), which were used to transform the BL21 derivative Rosetta (DE3) strain (Novagen). Stationary stage cultures were prepared by inoculating transformed Rosetta colonies in LB media and were then incubated overnight at 37° C. The following morning, stationary cultures were used to prepare large LB expression cultures, which were incubated at 37° C. while shaking until the expression cultures reached an OD600 nm of 0.7. Subsequently, they were induced with 0.5 mM IPTG and incubated at 37° C. for 4 hours. The cultures were then centrifuged and pellets were frozen at −80° C.

For the purification of the PR-10 allergens, bacterial pellets were lysed in BugBuster® HT Protein Extraction Reagent (Sigma) supplemented with EDTA-free protease inhibitor cocktail (Roche) for 1 hour at room temperature. Lysates were spun to remove debris, supernatants were harvested and filtered. His-tagged PR-10 proteins were purified with TALON® Superflow™ resins (Cytiva) in binding buffer (50 mM sodium phosphate, 300 mM NaCl, pH 7.4), following manufacturer's recommendations. Proteins were then dialyzed in 1×PBS overnight at 4° C.

### 2. Cross Reactivity to PR-10 Related Proteins by Direct Binding ELISA

ELISA plates were coated with anti-human-Fc-IgG overnight at 4° C. followed by capturing of MY010-mAbs (4E1, 25-3D3, 17B10) and REGN-mAbs (REGN-5713, REGN-5714, REGN-5715), respectively, (2 ug/ml) for 1 h at room temperature. Titrated his-tagged recombinant proteins (3 ug/mL-0) were incubated for 1 h at RT and binding was assessed by read-out with anti-His-tag-HRP.

Binding affinities (EC50) to the tested PR-10 allergens are shown in Table 5 below:

TABLE 5
Binding affinity (EC50) of MY010 antibodies and REGN antibodies to various PR-10 allergens.

| | | MY010 | | | REGN | | |
|---|---|---|---|---|---|---|---|
| | EC50 (ng/ml) | MY10-25-3D3 | MY010-17B10 | MY010-4E1 | REGN-5713 | REGN-5714 | REGN-5715 |
| PR-10 | rBet v 1.0101 | 3.051 | 2.877 | 3.253 | 3.44 | 4.237 | 4.055 |
| allergens | rCor a 1.0101 | 59.79 | 245.2 | 899.3 | n.b. | 616 | 73.86 |
| | rCor a 1.0201 | 4.545 | 9.354 | 5.567 | n.b. | 7.957 | 9.957 |
| | rAln g 1.0101 | 80.6 | 72.37 | 71.83 | n.b. | 532.4 | 144.4 |
| | rFag s 1.0101 | 81.49 | n.b. | 17.22 | n.b. | 127.5 | n.b. |
| | rQue a 1.0201 | n.b. | n.b. | 98.45 | n.b. | n.b. | n.b. |
| | rCas s 1.0101 | 1031 | n.b. | 6.938 | n.b. | n.b. | 79.2 |
| | rOst c 1.0101 | 34.47 | 107.8 | n.b. | 105.6 | 170.3 | n.b. |
| | rCar b 1.1A | 287 | 513.1 | n.b. | 757.5 | 1169 | n.b. |
| | rCar b 1.2 | 110 | 195.4 | 126.6 | 253.8 | n.b. | n.b. |

N.b.: not binding.

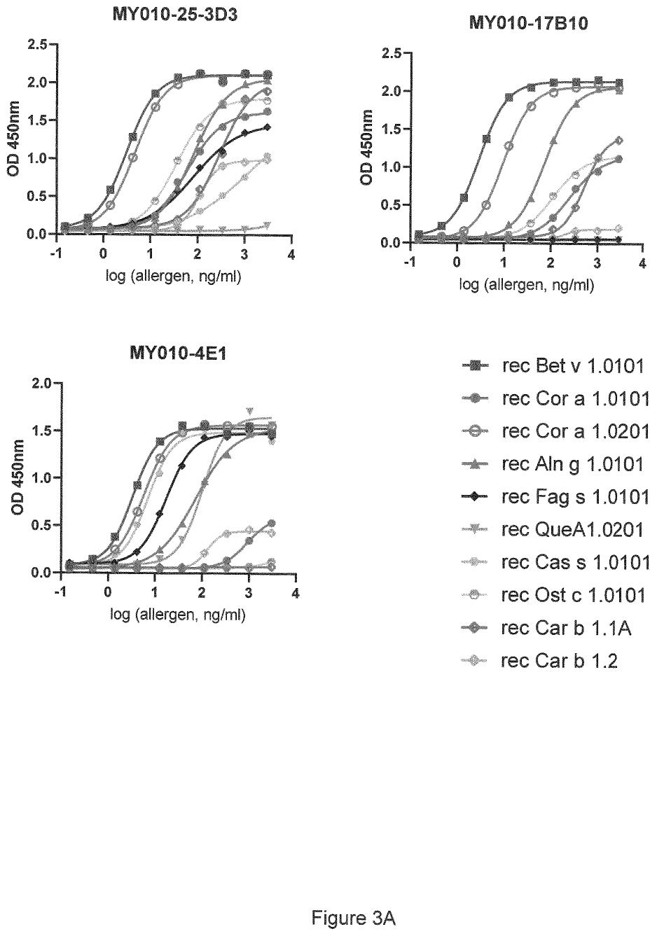

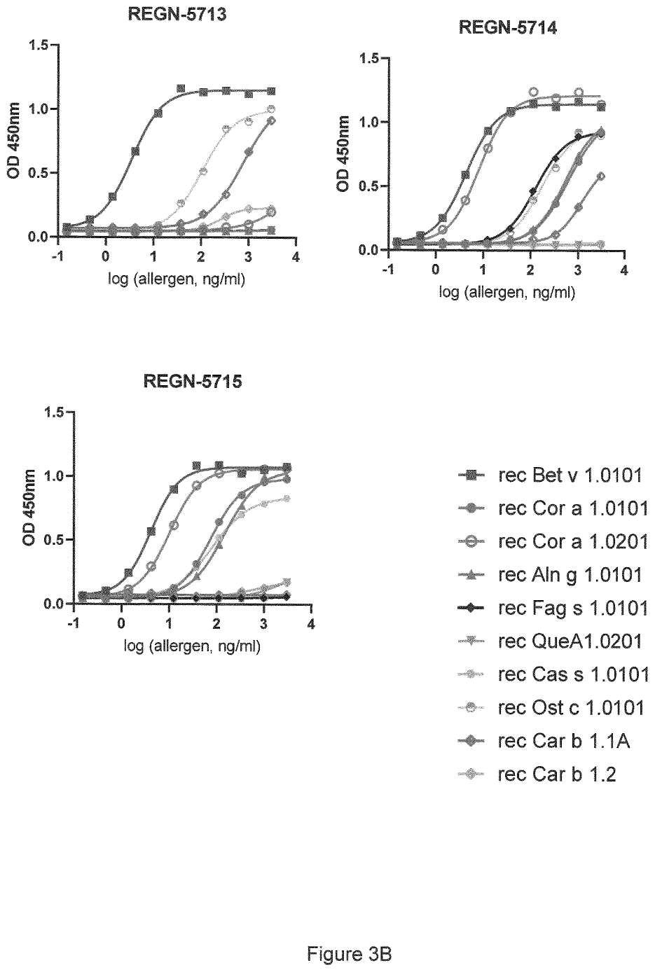

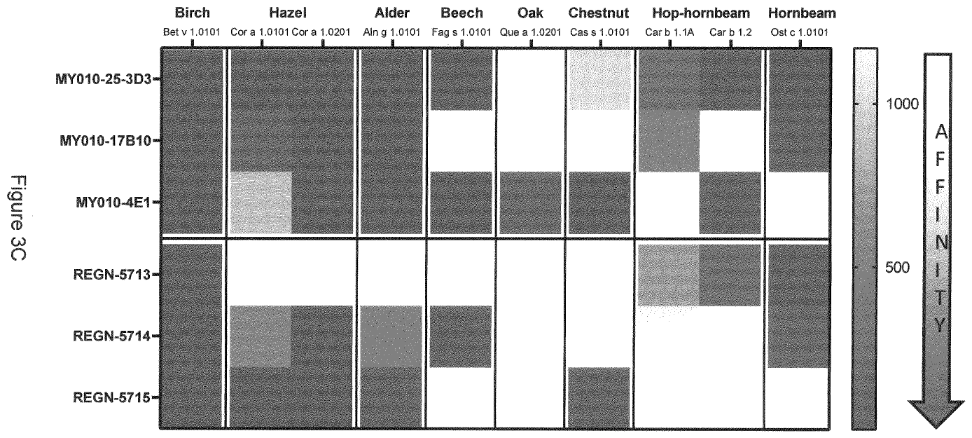

The binding affinities of 4E1, 25-3D3, and 17B10 to the various allergens are represented in FIG. 3A, while the binding affinities of REGN-5713, REGN-5714 and REGN-5715 are represented in FIG. 3B. The results are summarized in a heat map in FIG. 3C, where EC50 (ng/ml) is plotted.

Thus, FIGS. 3A to 3C as well as Table 5 demonstrate that MY010 antibodies 4E1, 25-3D3 and 17B10 are highly cross reactive to a number of recombinant PR-10 proteins from different tree pollen and show superior cross reactivity compared to prior art antibodies REGN-5713, REGN-5714 and REGN-5715.

### Example 4: Antibodies 4E1, 25-3D3 and 17B10 are Highly Cross-Reactive to Food Related PR-10 Allergens

### 1. Cross Reactivity to Food Related PR-10 Allergens by Direct Binding ELISA

Cross reactivity of MY010-antibodies 4E1, 25-3D3 and 17B10 as well as of prior art antibodies REGN-5713, REGN-5714 and REGN-5715 to PR-10 related food allergens (apple Mal d 1.0108, hazelnut Cor a 1.0401, and walnut Jug r 5.0101) was checked by soluble competition ELISA.

Therefore, plates were coated with streptavidin (1 ug/ml) overnight at 4° C. or 1 h at 37° C. followed by capturing of biotinylated na tBet v 1 (1.1 nM) overnight at 4° C. Titrated human MY010-mAbs (0-80 nM) and REGN-mABs, respectively, were preincubated with dilution buffer or respective PR-10 related food protein (Mal d 1, Cor a 1, Jug r 5; 14 nm and 140 nM) overnight at 4° C. or 1 h at RT. The pre-mixtures of mAbs/rec. proteins were incubated on the ELISA plate for 15 minutes at room temperature. Binding of MY010 antibodies to immobilized nat Bet v 1 was quantified by anti-hu-IgG-HRP. Source of PR-10 proteins: nat Bet v 1 (Indoor Biotechnologies), rec Mal d 1.0108 (Biomay), rec Cor a 1.0401 (Indoor Biotechnologies), rec Jug r 5.0101 (Indoor Biotechnologies).

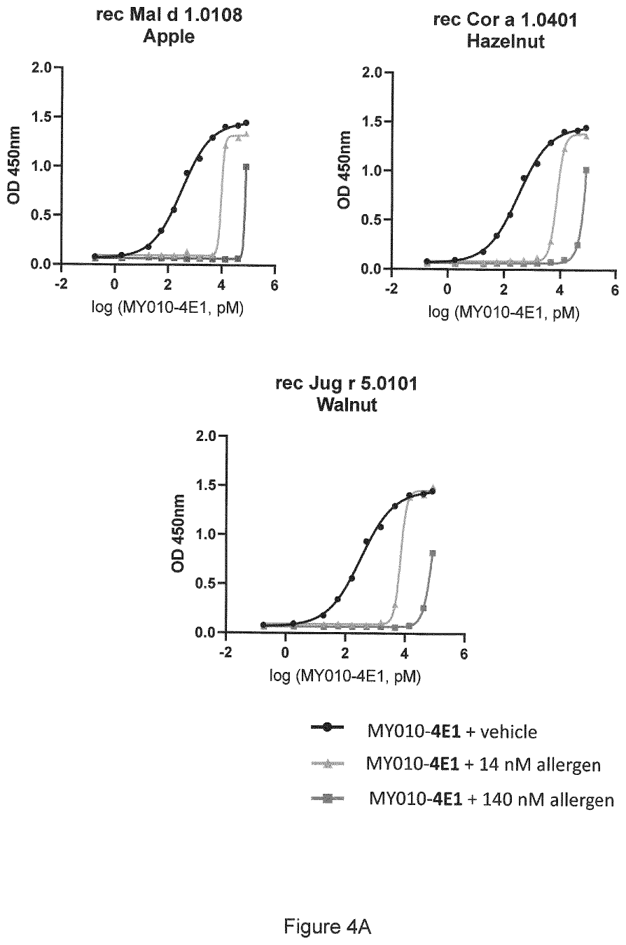

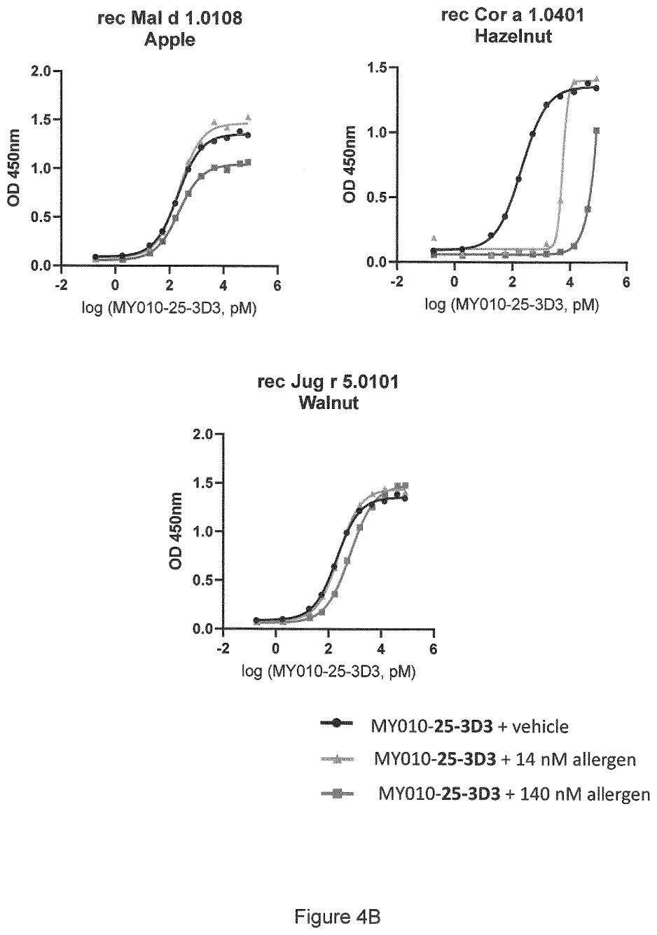

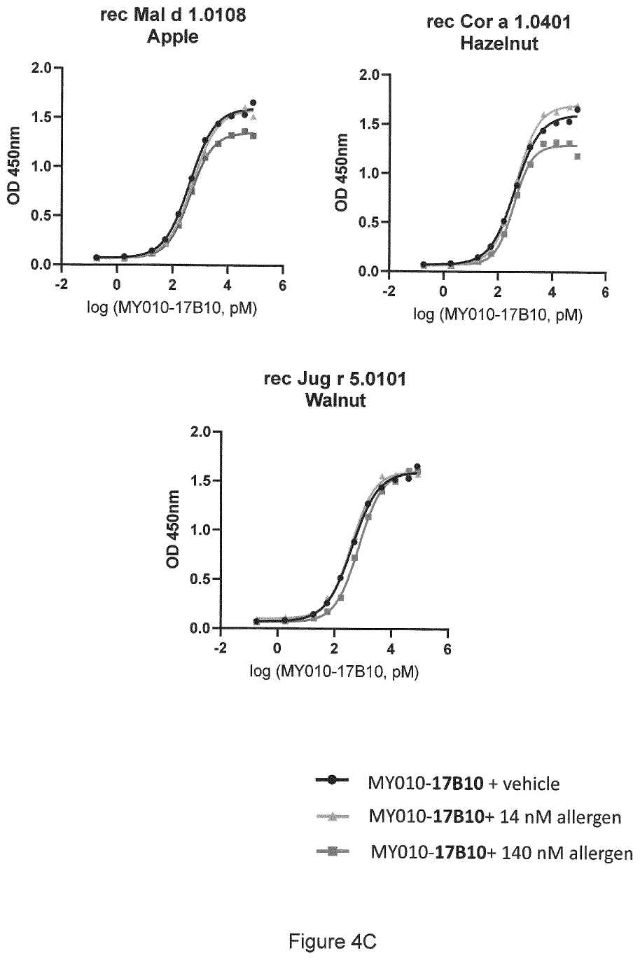

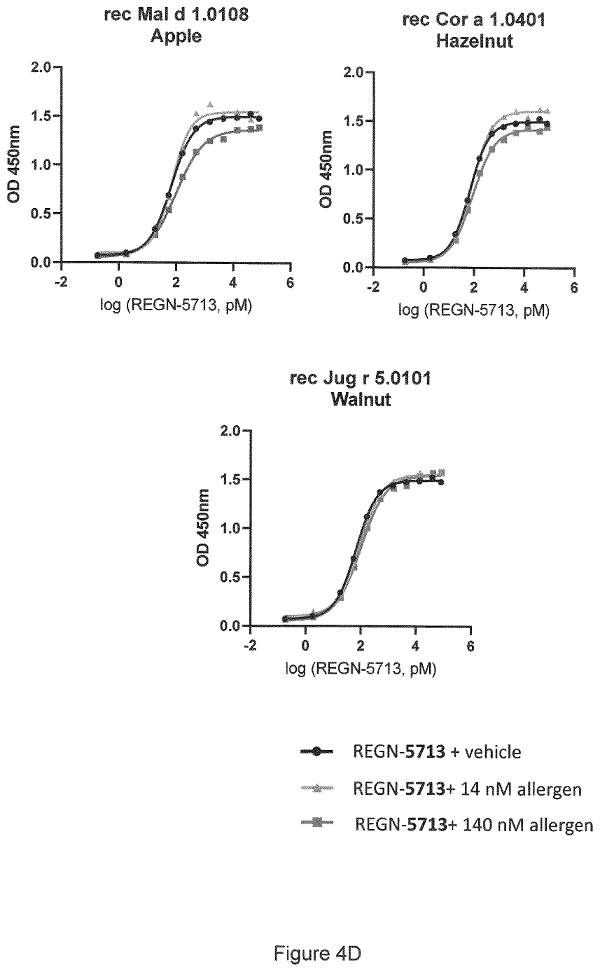

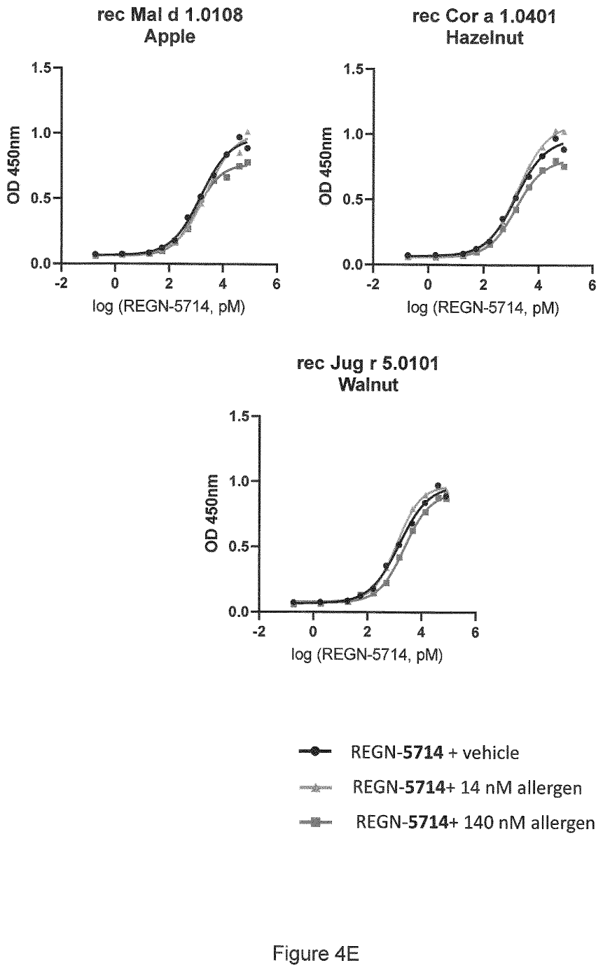

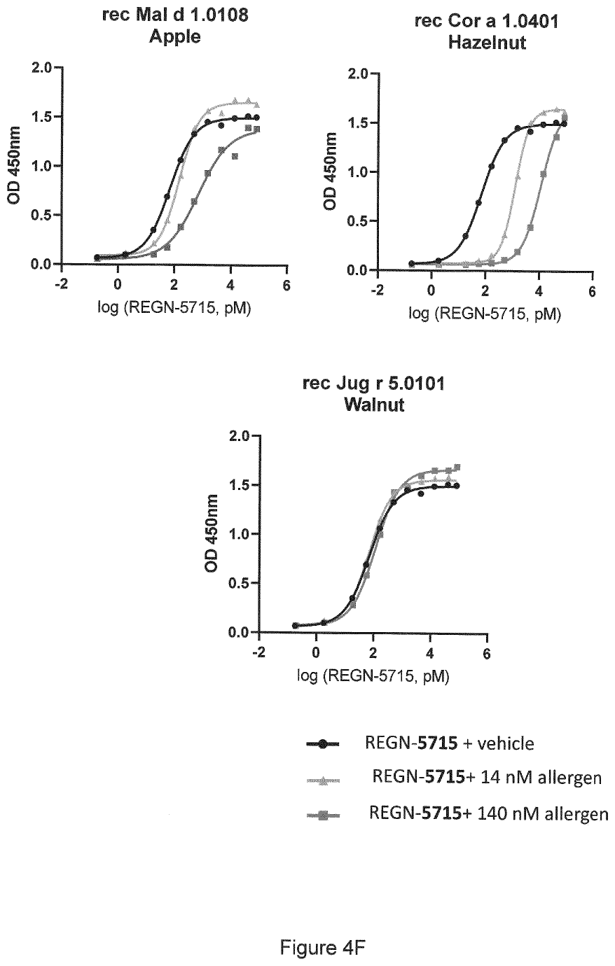

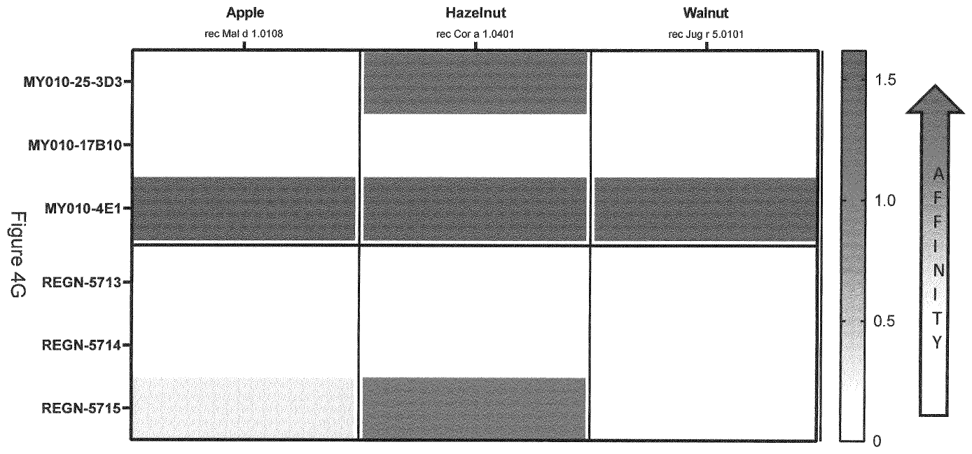

The results shown in FIG. 4A illustrate that MY010 antibody 4E1 binds to food related PR-allergens of apple (Mal d 1), hazelnut (Cor a 1) and walnut (Jur r 5) and therefore shows high cross-reactivity with respect to the major food related PR10 allergens. Also, MY010 antibody 25-3D3 has high affinity to hazelnut Cor a 1 (FIG. 4B).

On the other hand, as illustrated in FIGS. 4D to 4F, out of the prior art antibodies REGN-5713, REGN-5714 and REGN-5715, only REGN-5715 shows cross-reactivity with a food related allergen (hazelnut Cor a 1), and therefore has a lower level of cross-reactivity with respect to food related PR-10 allergens, in particular apple Mal d 1, hazelnut Cor a 1 and walnut Jug r 5, compared to the MY010 antibodies (4E1 and 25-3D3) of the present invention.

The results are summarized in the heat map of FIG. 4G where Δ Log EC50 is plotted: Δ Log EC₅₀=Log EC₅₀ (14 nM)−Log EC₅₀ (0 nM).

Thus, FIGS. 4A to 4G demonstrate the superior cross-reactivity of MY010 antibodies 4E1 and 25-3D3 compared to prior art antibodies REGN-5713, REGN-5714, REGN-5715 with respect to a number of food related PR-10 allergens.

### Example 5: Suppression of Mast Cell Responsiveness by Different MY010 Antibody Cocktails

Next, it was investigated whether different “cocktails” of MY010 antibodies, including one (4E1), two (4E1+25-3D3; 4E1+17B10) or three (4E1+17B10+25-3D3) MY010 antibodies, can inhibit allergen-mediated activation of bone marrow derived mast cells isolated from transgenic mice.

### 1. Comparison of Antibody Efficacy by Mast Cell Activation Test

Bone marrow derived mast cells isolated from transgenic mice expressing the human FcERIa are sensitized with plasma pool from birch pollen allergic patients. Activation of mast cells is quantified in terms of % CD107a positive cells. Cells are sensitized with plasma pool overnight at 37° C. and then stimulated for 35 minutes, at 37° C., with suitable allergen alone or previously pre-incubated with antibodies.

Titrated antibodies (starting at 320 nM) are pre-incubated with fixed concentration of extract. Fixed concentrations of allergens were chosen as follows: Birch extract: 7.41 ng/mL, Hazel pollen extract: 22 ng/mL, Black alder extract: 66.67 ng/mL, English oak extract 13.3 ng/mL, Beach extract: 13.3 ng/mL, Chestnut: 10.2 ng/mL, Hop-hornbeam: 200 ng/mL, Hornbeam: 67 ng/mL.

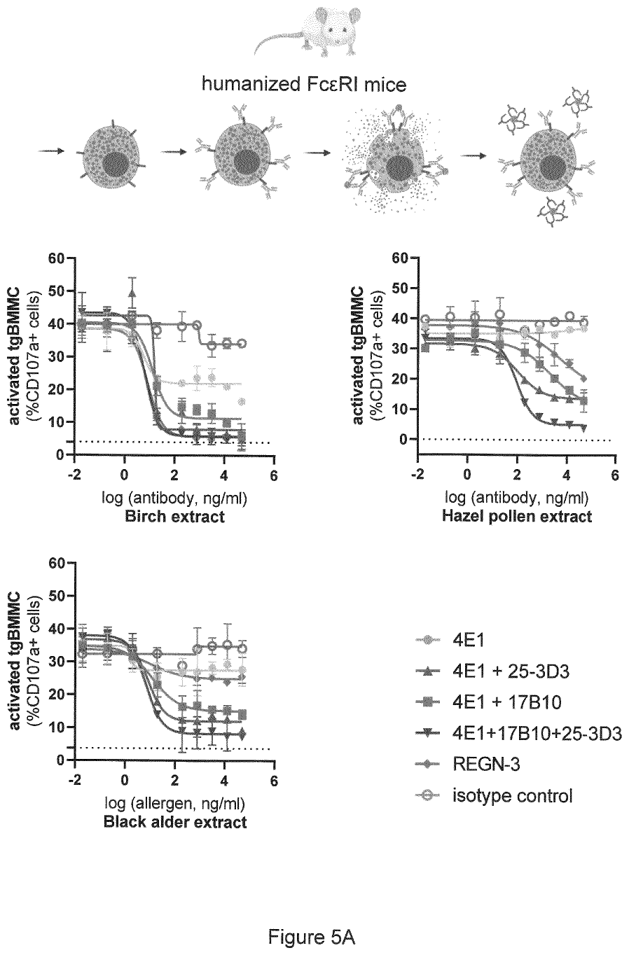

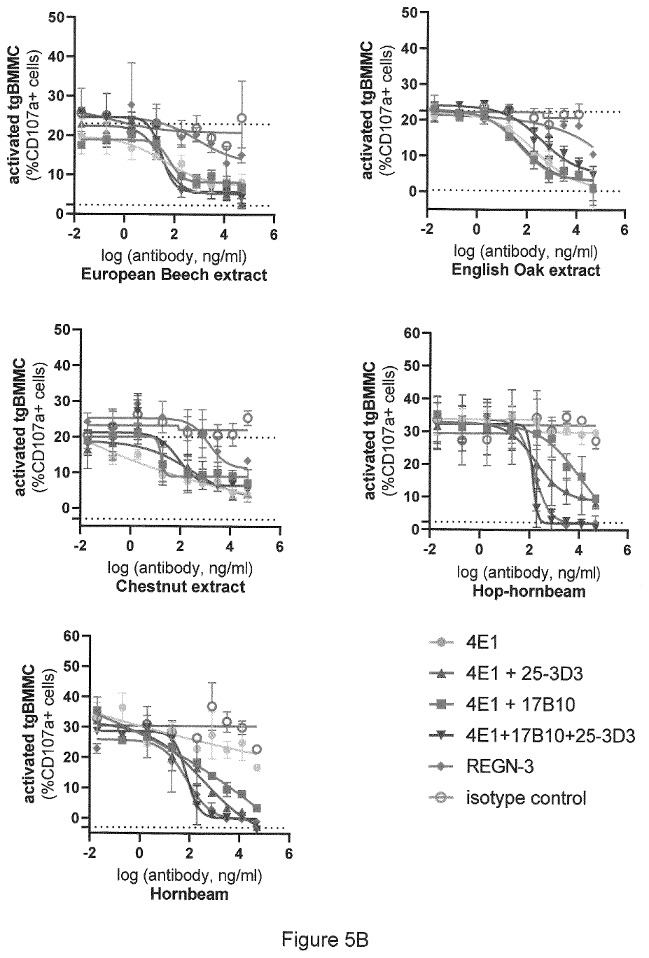

The results shown in FIGS. 5A and 5B illustrate that “cocktails” including one, two or three MY010 antibodies effectively suppress mast cell responsiveness in all birch-related extracts tested. In general, cocktails including two MY010 antibodies (4E1+25-3D3 or 4E1+17B10) are superior to cocktails including only one MY010 antibody (4E1), while cocktails including three antibodies (4E1+17B10+25-3D3) achieve the best cross-reactivity.

The results are summarized in Table 6 below:

TABLE 6
Inhibition of mass cell responsiveness

| | | | | | 4E1 + | |
|---|---|---|---|---|---|---|
| | | | 4E1 + | 4E1 + | 25-3D3 + | |
| | | 4E1 | 17-B10 | 25-3D3 | 17B10 | REGN-3 |
| Birch | IC50 (ng/mL) | 5.49 | 12.64 | 15.11 | 6.26 | 8.15 |
| | % inhibition | 56.6 | 83.8 | 86.3 | 85.8 | 84.6 |
| Hazel | IC50 (ng/mL) | 35259.00 | 2029.00 | 98.14 | 89.73 | 5008.00 |
| | % inhibition | −9.6 | 61.3 | 59.1 | 88.6 | 39.7 |
| Alder | IC50 (ng/mL) | 1.72 | 11.82 | 7.91 | 6.60 | 19.76 |
| | % inhibition | 23.3 | 61.0 | 74.8 | 79.7 | 29.4 |
| Oak | IC50 (ng/mL) | 208.10 | 47.73 | 65.45 | 373.00 | 2.732E+16 |
| | % inhibition | 80.6 | 80.5 | 78.9 | 79.6 | 54.4 |
| Beech | IC50 (ng/mL) | 39.06 | 64.14 | 31.09 | 29.64 | 882.40 |
| | % inhibition | 65.8 | 71.1 | 87.9 | 82.3 | 37.1 |
| Chestnut | IC50 (ng/mL) | 40.08 | 16.18 | 254.90 | 81.23 | 1344.00 |
| | % inhibition | 83.3 | 68.5 | 81.9 | 77.3 | 41.3 |
| Hop- | IC50 (ng/mL) | 219.30 | 9301.00 | 171.90 | 127.50 | 149.50 |
| hornbeam | % inhibition | 8.4 | 70.1 | 74.4 | 98.0 | 93.1 |
| Hornbeam | IC50 (ng/mL) | n.c. | 626.00 | 341.20 | 71.04 | 75.09 |
| | % inhibition | 39.38 | 95.2 | 107.1 | 104.8 | 101.3 |

% inhibition calculated at 48 ug/mL of antibody: 100 − (% CD107a)_{48 ug/mL Ab}/(% CD107a)_{0 ug/mL Ab} * 100.
N.c.: not calculable.

### Example 6: MY010 Cocktail Shows Efficacy in all Birch-Related Extracts Tested

Next, the ability of a MY010 cocktail including three antibodies (4E1+1 7B110±25-3D3) to inhibit allergen-mediated activation of bone marrow derived mast cells isolated from transgenic mice was compared to a prior art cocktail containing three prior art antibodies (REGN13-15).

### 1. Comparison of Antibody Efficacy by Mast Cell Activation Test

Bone marrow derived mast cells isolated from transgenic mice expressing the human FcERIa are sensitized with plasma pool from birch pollen allergic patients. Activation of mast cells is quantified in terms of % CD107a positive cells. Cells are sensitized with plasma pool overnight at 37° C. and then stimulated for 35 minutes, at 37° C., with suitable allergen alone or previously pre-incubated with antibodies.

Titrated allergens/extracts are pre-incubated with fixed concentration of antibodies (80 nM).

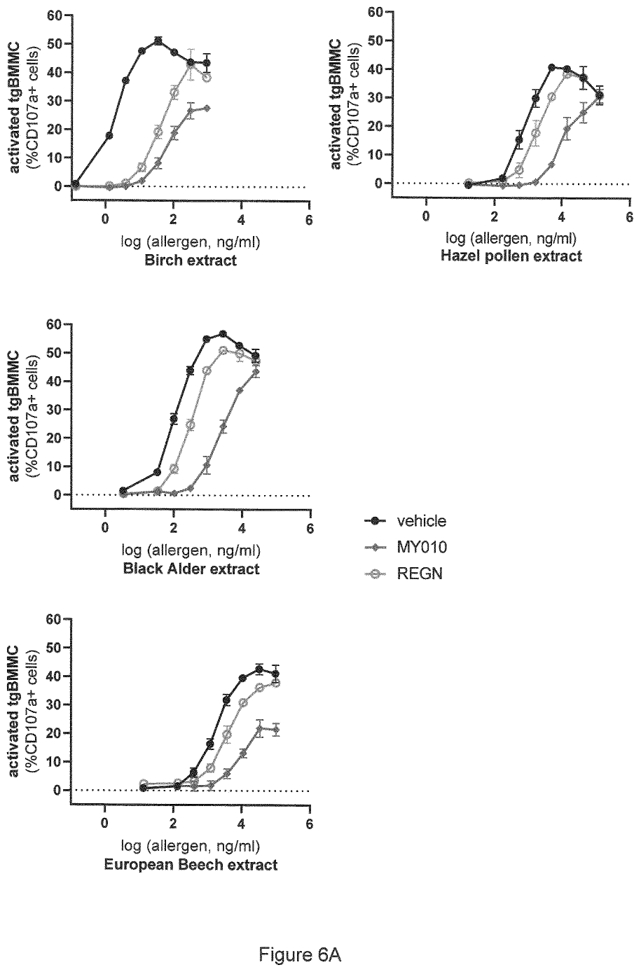

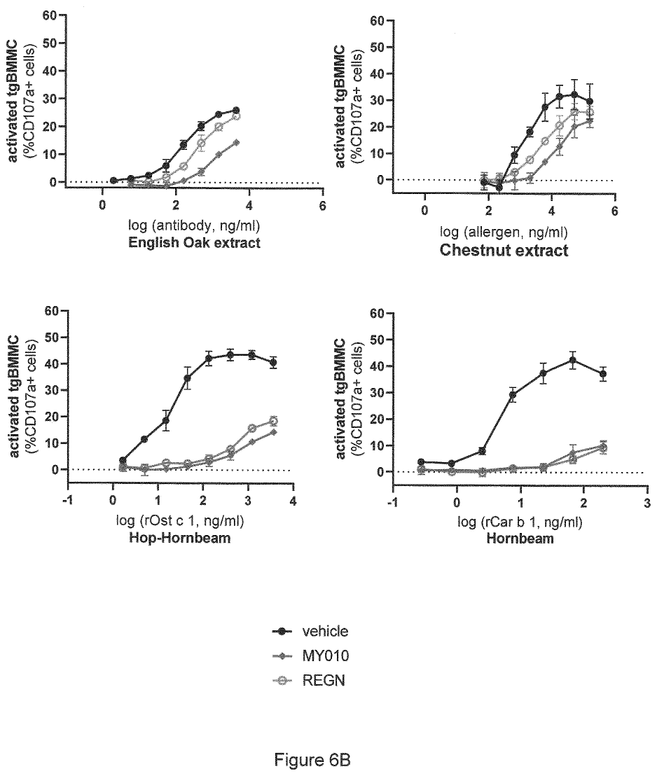

The results shown in FIGS. 6A and 6B illustrate that the MY010 cocktail including the inventive antibodies 4E1+17B10+25-3D3 shows efficacy in all birch-related extracts/allergens tested, and is superior to the prior art REGN-cocktail.

### Example 7: Cocktail of Antibodies 4E1, 17B10 and 25-3D3 Inhibits Allergen-Mediated Activation of Basophils Derived from Allergic Patients

Next, it was investigated whether the cocktail of antibodies 4E1, 17B10 and 25-3D3 can inhibit allergen-mediated activation of basophils derived from allergic patients.

To this end, a whole blood basophil activation test was performed. Briefly, heparinized whole blood from birch pollen allergic donors was either left untreated or stimulated for 30 minutes at 37° C. with birch extract in absence or in the presence of the cocktail of antibodies (80 nM) 4E1, 17B10 and 25-3D3 (pre-incubation birch extract/antibodies performed at room temperature for at least 1 h). Before erythrocyte lysis, cells were stained with CCR3-PE and CD63-FITC. Basophils were gated as SSClow, CCR3high lymphocytes and activation was quantified using % of CD63+, CCR3high cells using FACS Calibur (Becton Dickinson AG, Allschwil, Switzerland). The flow cytometry data was analyzed using Flowjo software (TreeStar, Ashland, Ore).

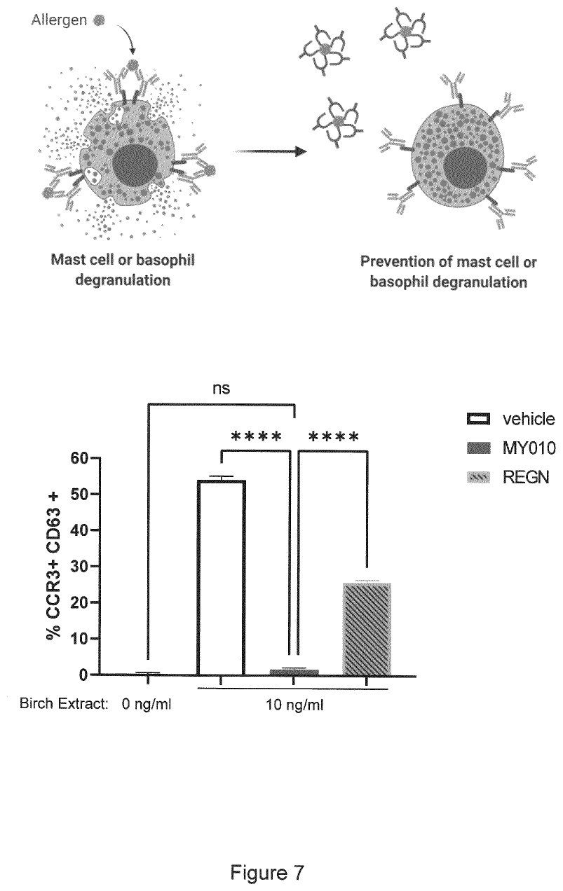

The results are presented in FIG. 7. Bars indicate activation of basophils expressed as % CD63+ cells (mean±s.d, n-2). White bars: no addition of antibody. Data from 1 representative patient. Statistics done by One way anova followed by Dunnett's test comparing each column to 10 ng/ml birch extract.

In summary, these data show that pre-incubation of birch extract with the MY010 antibody cocktail decreased basophil degranulation, and that the MY010 cocktail (4E1, 17B10 and 25-3D3) is superior in inhibiting allergen-mediated activation of basophils derived from allergic patients than the prior art REGN cocktail (REGN-5713, REGN-5714, REGN-5715).

### Example 8: Cocktail of Antibodies 4E1, 17B10 and 25-3D3 Shows Efficacy on Birch Related Food Allergens

The efficacy of MY010 antibody cocktail (4E1, 17B10 and 25-3D3) on birch related food allergens was tested by means of a mast cell activation test.

Bone marrow derived mast cells isolated from transgenic mice expressing the human FcERIa are sensitized with plasma pool from birch pollen allergic patients. Activation of mast cells is quantified in terms of % CD107a positive cells or in terms of mediator release (sLts, Bühlmann CAST ELISA). Cells are sensitized with plasma pool overnight at 37° C. and then stimulated for 35 minutes, at 37° C., with suitable allergen alone or previously pre-incubated with antibodies.

Titrated allergen/extracts (apple, hazelnut, walnut, peach) are pre-incubated with fixed concentration of antibodies (240 nM).

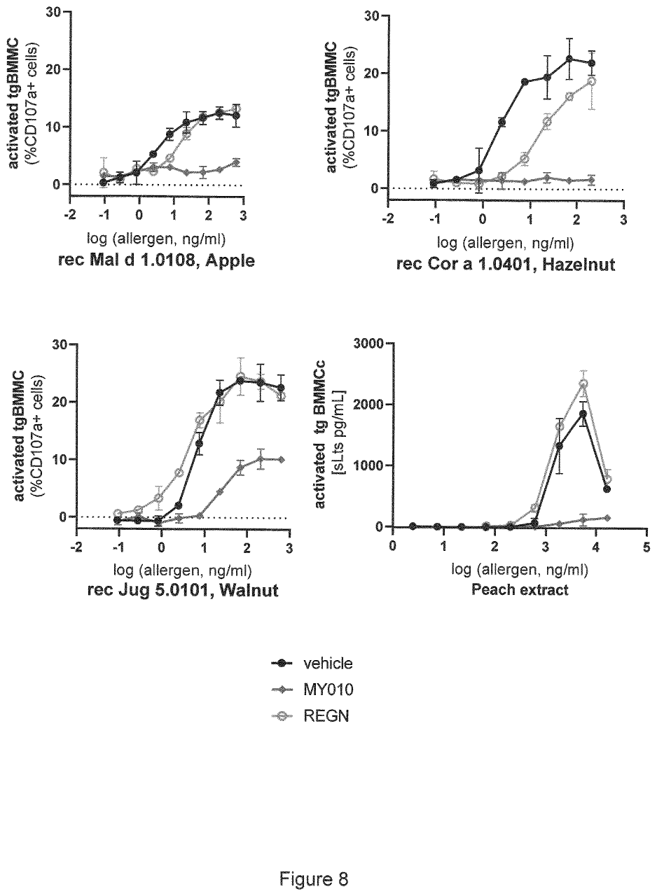

The results shown in FIG. 8 illustrate that cocktails including the MY010 antibodies 4E1, 17B110 and 25-3D3 effectively suppress mast cell responsiveness in all birch-related food allergens/extracts tested. In each case, cocktails including the MY010 antibodies (4E1, 25-3D3 and 17B10) are superior to cocktails including the REGN antibodies (REGN-5713, REGN-5714 and REGN-5715).

### Example 9: Antibodies 6C9, 3F1 and 13A2 Belong to the Same Clonotype and Compete with Each Other and with Antibody 17B10

Antibodies 6C9, 3F1 and 13A2 were cloned and expressed as described in Example 1 (cf. 1. Antibody cloning; and 2. Antibody Expression and Purification).

CDR and VH/VL sequences (SEQ ID NOs) of fully human antibodies 6C9, 3F1 and 13A2 are shown in Table 7 below:

TABLE 7
SEQ ID NOs for CDR and VH/VL sequences of antibodies 6C9, 3F1 and 13A2

| Antibody | CDRH1 | CDRH2 | CDRH3 | CDRL1 | CDRL2 | CDRL3 | VH | VL |
|---|---|---|---|---|---|---|---|---|
| 6C9 | 66 | 67 | 68 | 69 | 70 | 71 | 72 | 73 |
| 3F1 | 74 | 75 | 76 | 77 | 78 | 79 | 80 | 81 |
| 13A2 | 82 | 83 | 84 | 85 | 86 | 87 | 88 | 89 |

To determine whether antibodies 6C9, 3F1 and 13A2 compete with antibodies 4E1, 25-3D3 and 17B10 (MY010 antibodies), a cross-competion ELISA assay was performed on Bet v 1. To this end, plates were coated with MY010-IgG antibodies (all at 0.75 ug/mL) overnight at 4° C. MY010 as Fabs (160 nM) were pre-incubated with recBet v 1-his-tag (0.05 ug/ml=2.38 nM) for at least 1 h at RT. Then, the antibody-allergen pre-mixtures were incubated on ELISA plates (2 h, RT). Read-out was performed with anti-His-HRP. On the table shown in FIG. 9, the relative % of inhibition at the highest antibody-concentration is plotted. % of inhibition=100−(OD450Ab/OD450w/o Ab*100) where OD450Ab is the OD450 at each point of the titration curve and OD450w/o Ab is the mean of the OD450s reached without competing antibody. The relative percentage of inhibition is then calculated by normalizing to the maximum inhibition reached with the competition of the coated antibody with itself. The results are summarized in FIG. 9.

FIG. 9 shows that antibodies MY010-3F1, MY010-6C9 and MY010-13A2 compete with each other and also compete with MY010-17B10. Based on this and the sequence similarities in the VH and VL regions, it can be concluded that they belong to the same clonotype. Additionally, this clonotype belongs to the same complementation group of MY010-17B10. On the other hand, they do not compete with the antibodies from the other complementation groups (25-3D3 and 4E1).

### Example 10: Antibodies 4E1, 25-3D3 and 3F1 Bind to Distinct Non-Overlapping Epitopes on Bet v 1

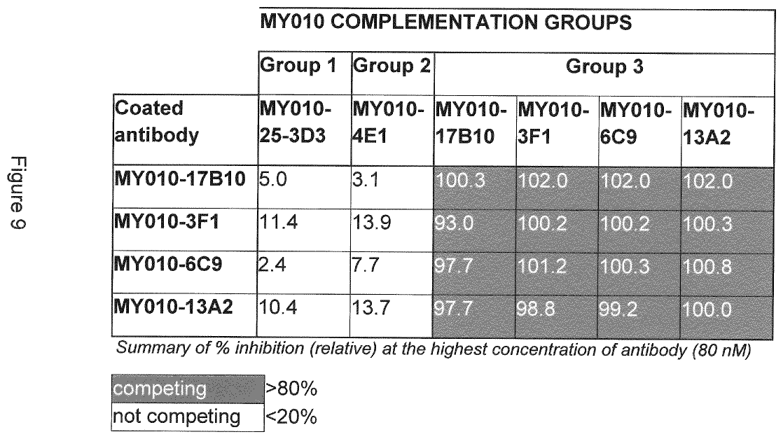

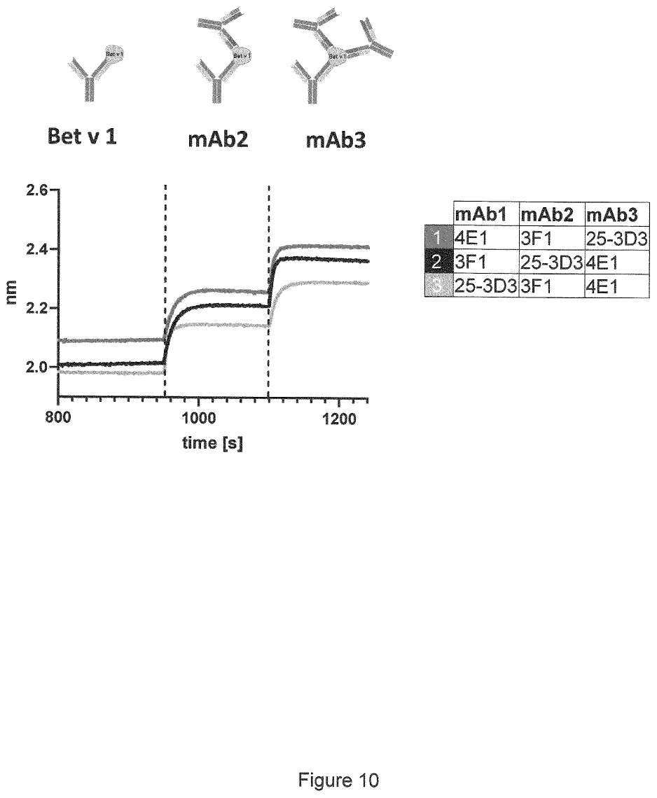

To determine whether antibodies 4E1, 25-3D3 and 3F1, as representor of the 6C9 clonotype group, can bind simultaneously to the epitopes on Bet v 1, biolayer interferometry (BLI; Forte Bio Octet RED96e; Running buffer: 1×PBS) was performed. IgG4 of the cocktail mAbs were used. First, mAbs1 were immobilized (5 ug/ml) on a human Fc binding BLI sensor surface, which were subsequently blocked with human Fc fragment (50 ug/ml). Then natural Bet v 1 (1.8 ug/ml) was applied followed by sequential injection of mAbs2 and mAbs3 (each 15 ug/ml), as indicated in the Table of FIG. 10.

The sensogram shown in FIG. 10 strongly suggests that all the IgGs can bind at the same time to one Bet v 1 molecule. Thus, the BLI data confirmed that all tested IgG antibodies 4E1, 25-3D3 and 3F1, a representor of the 6C9 clonotype group, bind simultaneously and non-competitively to a single Bet v 1 molecule. Accordingly, these data demonstrate that 4E1, 25-3D3 and 3F1 antibodies bind to distinct, non-overlapping epitopes on Bet v 1.

### Example 11: Antibodies 6C9, 3F1 and 13A2 Bind to PR-10 Allergen Extracts with High Affinity

Cross reactivity of MY010 antibodies 6C9, 3F1, and 13A2, as well as of MY010-17B10, belonging to the same complementation group, to PR-10 related extracts (birch, hazel, black alder, American beech, oak) was checked by soluble competition ELISA.

Therefore plates were coated with streptavidin (1 ug/ml) overnight at 4° C. or 1 h at 37° C. followed by capturing of biotinylated natBet v 1 (1.4 nM) overnight at 4° C. Titrated human MY010-mAbs (6C9, 3F1, 13A2, 17B10) (80 nM-0 nM) were preincubated with dilution buffer (no extract condition=0 nM) or 14 nM of extract overnight at 4° C. or 1 h at RT. The pre-mixtures of MY010-mAbs/extract were incubated on the ELISA plate for 15 minutes at room temperature. Read-out was accomplished by anti-hu-IgG-HRP.

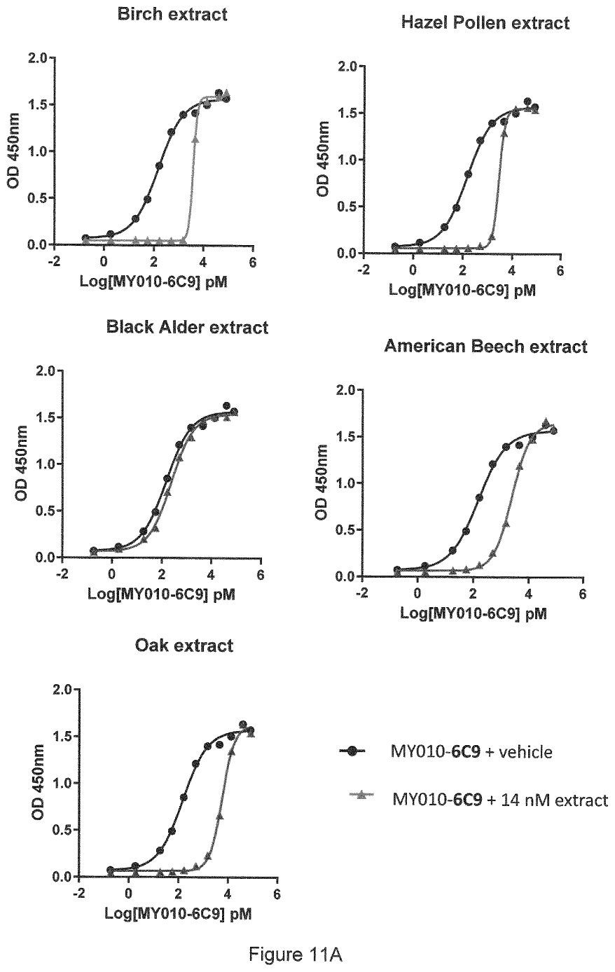

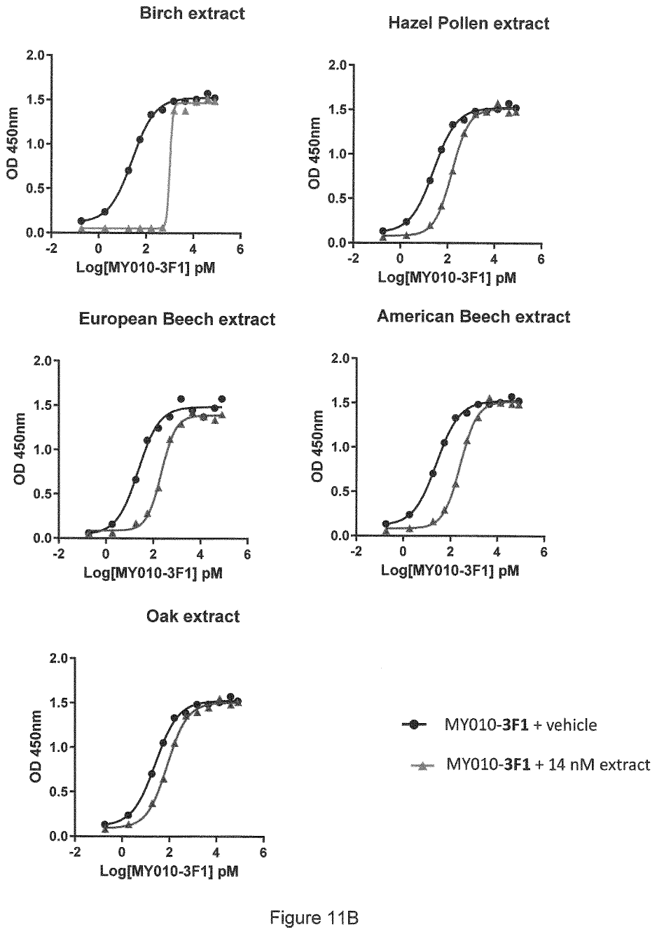

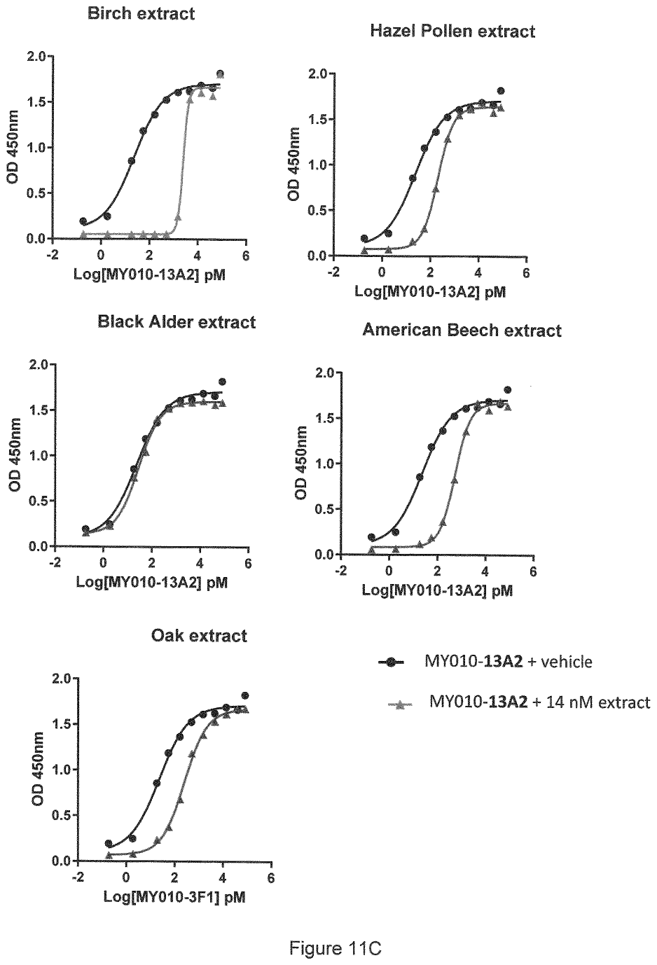

The results shown in FIG. 11A (mAb 6C9), FIG. 11B (mAb 3F1) and FIG. 11C (mAb 13A2) illustrate that MY010 antibodies 6C9, 3F1 and 13A2 bind to PR-10 allergen extracts of birch, hazel, beech and oak with high affinity and therefore show a broad cross-reactivity with respect to a wide spectrum of tree pollen allergens.

On the other hand, as illustrated in FIGS. 2D to 2F, the prior art mAbs REGN-5713 (FIG. 2D), REGN-5714 (FIG. 2E) and REGN-5715 (FIG. 2F) show a lower level of cross-reactivity with respect to birch-related PR-10 allergen extracts, in particular hazel, beech and oak extracts. Thus, FIGS. 11A to 11C demonstrate the superior cross-reactivity of MY010 antibodies 6C9, 3F1, and 13A2 compared to prior art antibodies REGN-5713, REGN-5714, REGN-5715 with respect to a number of birch related tree pollen extracts.

Moreover, comparison with FIG. 2C shows that 6C9, 3F1 and 13A2 antibodies possess better cross-reactivity to oak compared to antibody 17B110 of the same complementation group.

The results are summarized in the heat map of FIG. 11D, where Δ Log EC50 is plotted: Δ Log EC₅₀=Log EC₅₀(14 nM)−Log EC₅₀(0 nM).

### Example 12: Antibodies 6C9, 3F1 and 13A2 are Highly Cross-Reactive to Birch Related Recombinant PR-10 Proteins

In this experiment, a number of Bet v 1 related (PR-10) allergens synthesized according to Example 3 were used to define affinities of MY010 antibodies of the 6C9 clonotype (6C9, 3F1 and 13A2) on birch related recombinant proteins Bet v 1.0101 (birch); Cor a 1.0101 and Cor a 1.0201 (hazel); Ain g 1.0101 (black alder); Fag s 1.0101 (beech); Que a 1.0201, Que a 1.0301 (oak); Cas s 1.0101 (chestnut); Car b 1.1A, Car b1.2 (hornbeam); and Ost c 1.0101 (hop-hornbeam) by direct binding ELISA.

ELISA plates were coated with anti-human-Fc-IgG overnight at 4° C. followed by capturing of MY010-mAbs (6C9, 3F1 and 13A2) (2 ug/ml) for 1 h at room temperature. Titrated his-tagged recombinant proteins (3 ug/mL-0) were incubated for 1 h at RT and binding was assessed by read-out with anti-H is-tag-HRP.

Binding affinities (EC50) to the tested PR-10 allergens are shown in Table 8 below:

TABLE 8
EC50s (ng/mL) for 6C9-clonotype determined by ELISA;

| | | 3F1 | 6C9 | 13A2 |
|---|---|---|---|---|
| PR-10 allergens | Bet v 1 | 4.496 | 4.264 | 4.321 |
| | Cor a 1.0101 | n.b | 168.6 | 116.9 |
| | Cor a 1.0201 | 9.648 | 7.517 | 10.74 |
| | Aln g 1 | n.b | 1623 | 669.2 |
| | Fag s 1.0101 | 97.69 | 35.96 | 32.6 |
| | Que a 1.0201 | n.b | 2366 | 1421 |
| | Que a 1.0301 | 2121 | 19.02 | 64.14 |
| | Cas s 1.0101 | 31.71 | 8.75 | 17.66 |
| | Car b 1.1° | n.b | 1516 | 3208 |
| | Car b 1.2 | n.b | 138.9 | 194.6 |
| | Ost c 1.0101 | n.b | 93.47 | n.b |

n.b.: not binding

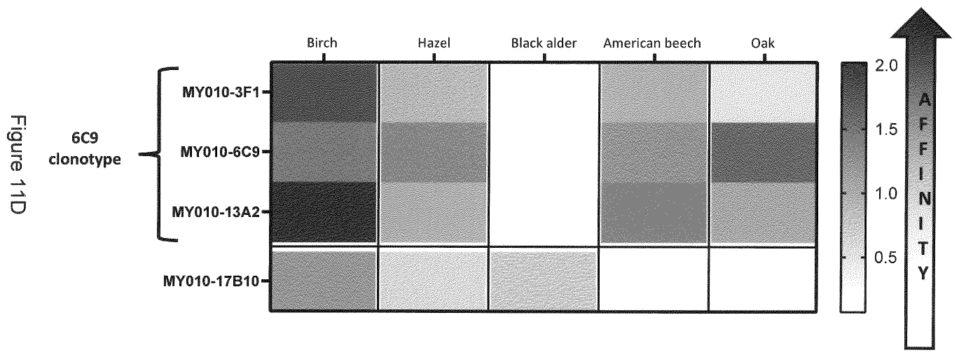

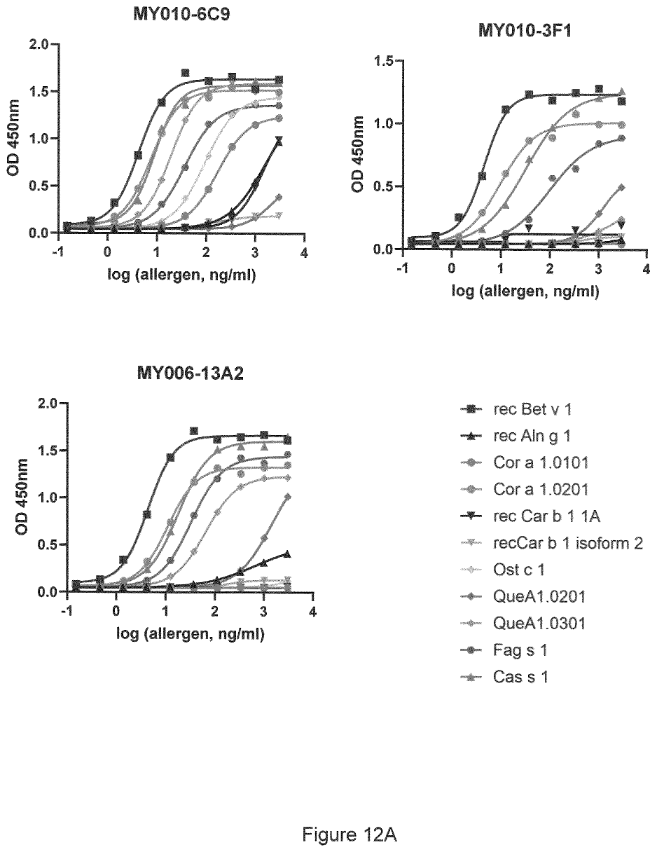

The binding affinities of 6C9, 3F1 and 13A2 to the various allergens are represented in FIG. 12A. The results are summarized in a heat map in FIG. 12B, where EC50 (ng/ml) is plotted.

Thus, FIGS. 12A and 12B as well as Table 8 demonstrate that MY010 antibodies 6C9, 3F1 and 13A2 are highly cross reactive to a number of recombinant PR-10 proteins from different tree pollen and show superior cross reactivity compared to prior art antibodies REGN-5713, REGN-5714 and REGN-5715 (cf. FIGS. 3B and 3C). Moreover, MY010 antibodies 6C9, 3F1 and 13A2 have a higher binding affinity in comparison to antibody MY010-17B10, belonging to the same complementation group, (cf. FIGS. 3A and 12B). Out of MY010 antibodies 6C9, 3F1 and 13A2, MY010-6C9 is the most cross reactive antibody.

### Example 13: Antibodies 6C9, 3F1 and 13A2 are Cross-Reactive to Food Related Walnut allergens

### 1. Cross Reactivity to Food Related PR-10 Allergens by Direct Binding ELISA

Cross reactivity of MY010-antibodies 6C9, 3F1 and 13A2 to several PR-10 related food allergens (e.g. apple Mal d 1.0108, hazelnut Cor a 1.0401, and walnut Jug r 5.0101) was checked by soluble competition ELISA.

Therefore, plates were coated with streptavidin (1 ug/ml) overnight at 4° C. or 1 h at 37° C. followed by capturing of biotinylated na tBet v 1 (1.4 nM) overnight at 4° C. Titrated human MY010-mAbs (0-80 nM) were preincubated with dilution buffer or respective recombinant PR-10 related food protein (14 nm and 140 nM) overnight at 4° C. or 1 h at RT. The pre-mixtures of mAbs/rec. proteins were incubated on the ELISA plate for 15 minutes at room temperature. Binding of MY010 antibodies to immobilized nat Bet v 1 was quantified by anti-hu-IgG-HRP.

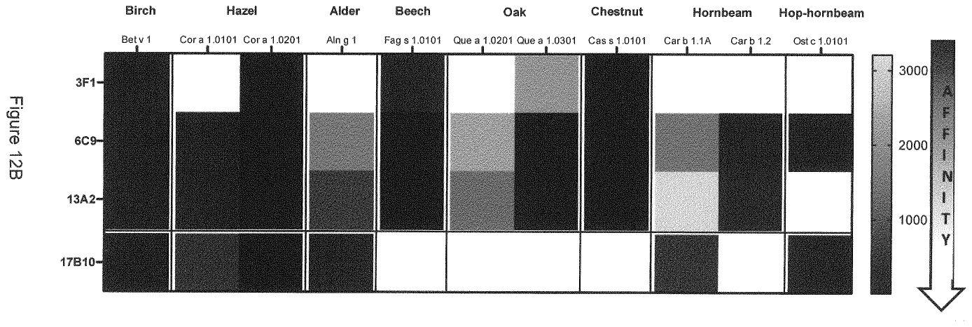

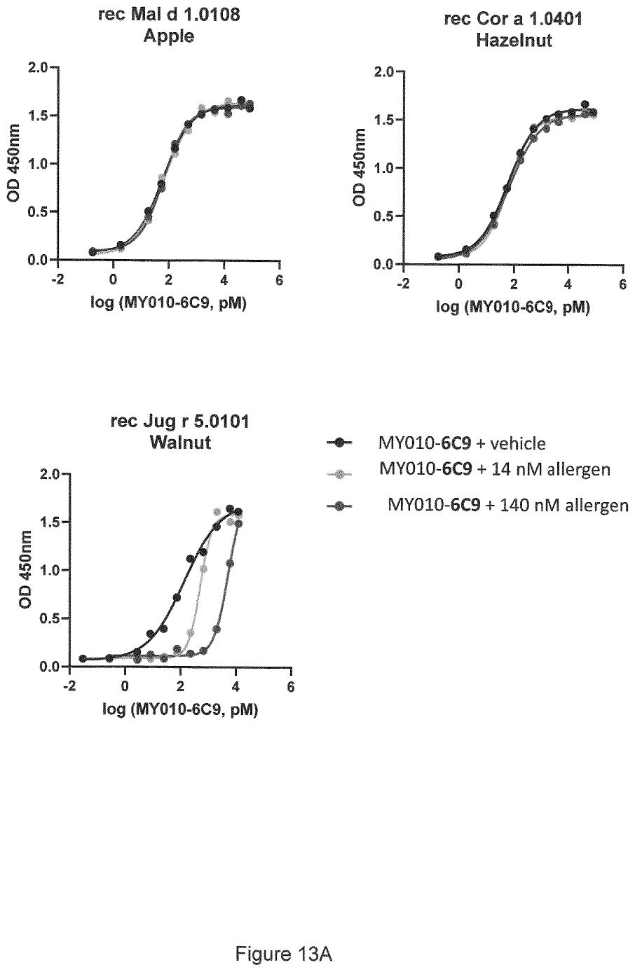

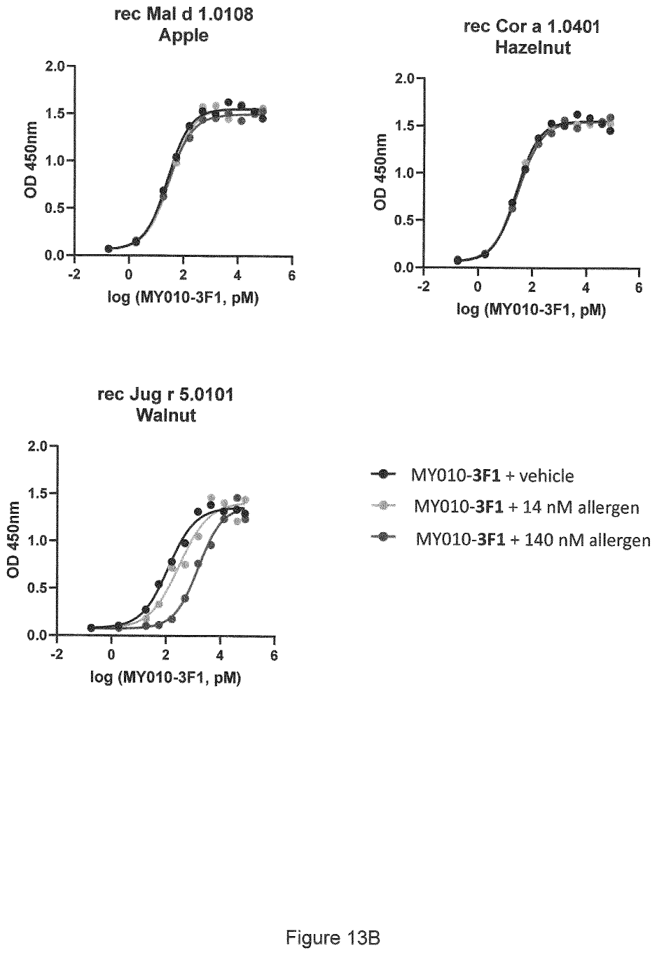

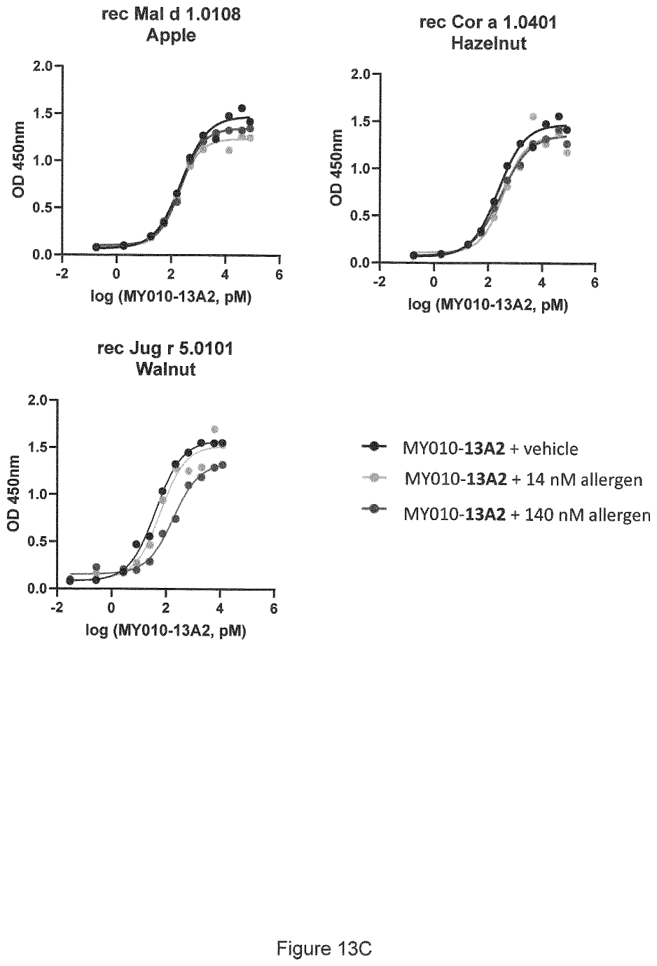

As shown in FIGS. 13A to 13C, MY010 antibodies 6C9, 3F1 and 13A2 have affinity to food related walnut allergen Jur r 5, and thus show superior cross-reactivity with respect to the walnut allergen compared to antibody 17B10 belonging to the same complementation group (cf. FIG. 4C), and also in comparison to the prior art antibodies REGN-5713, REGN-5714 and REGN-5715 (cf. FIGS. 4D to 4F).

### Example 14: Suppression of Mast Cell Responsiveness by Different MY010 Antibody Cocktails

Next, it was investigated whether different “cocktails” of MY010 antibodies, each including three different MY010 antibodies (17B10+4E1+25-3D3; 6C9+4E1+25-3D3; 3F1+4E1+25-3D3), can inhibit allergen-mediated activation of bone marrow derived mast cells isolated from transgenic mice.

### 1. Comparison of Antibody Efficacy by Mast Cell Activation Test

Bone marrow derived mast cells isolated from transgenic mice expressing the human FcERIa are sensitized with plasma pools from birch pollen allergic patients from USn (n=6 patients, from UK (n=5 patients) and from Switzerland (n=10 patients). Activation of mast cells is quantified in terms of % CD107a positive cells or in terms of mediator release (sLts, Bühlmann CAST ELISA). Cells are sensitized with plasma pool overnight at 37° C. and then stimulated for 35 minutes, at 37° C., with suitable allergen alone or previously pre-incubated with antibodies. Titrated allergen/extracts of birch, black alder, hazel pollen, white oak, beech, hop-hornbeam and hornbeam, respectively, are pre-incubated with fixed concentration of antibodies.

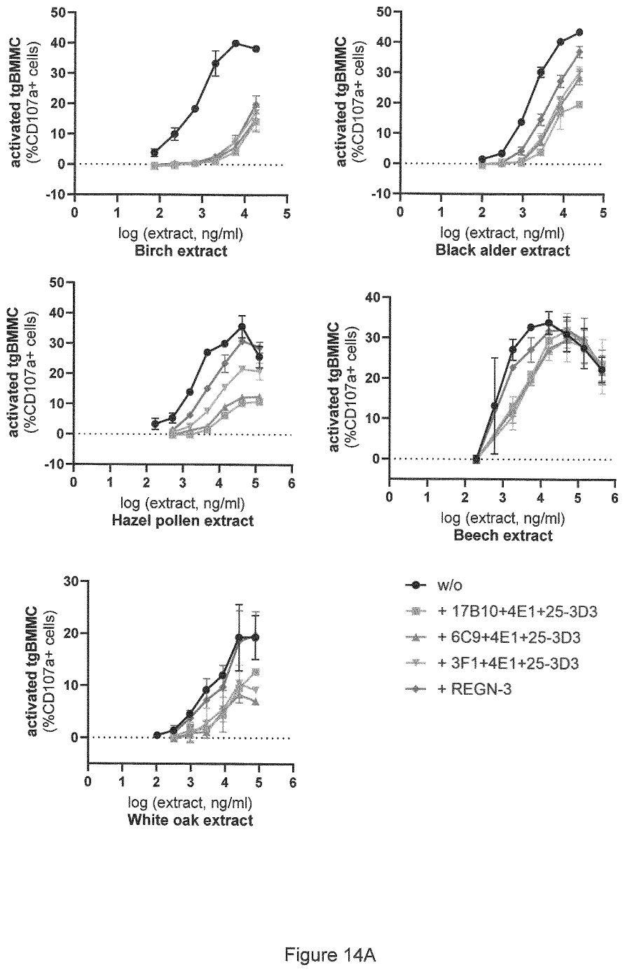

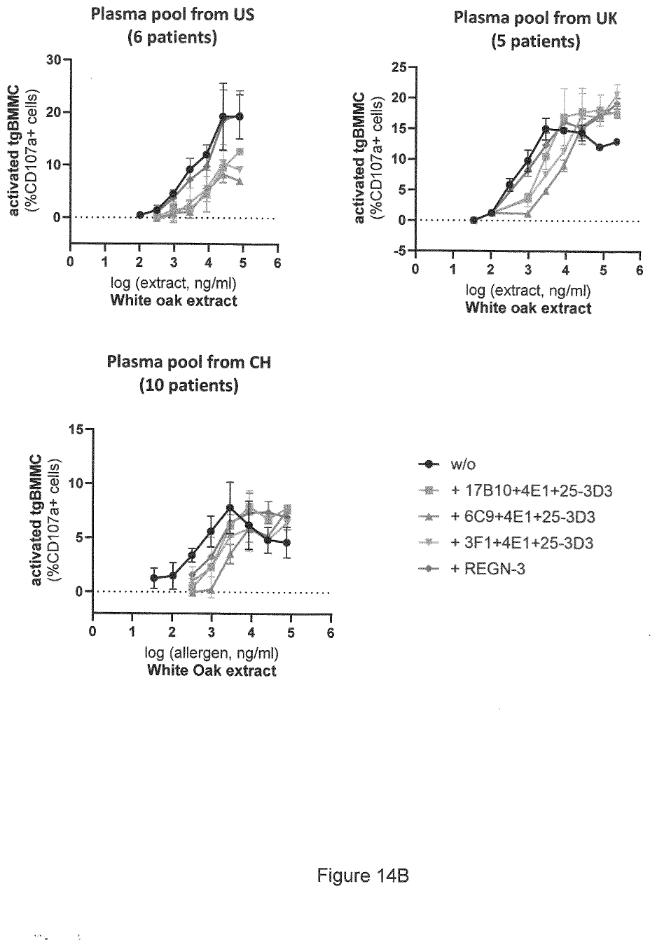

The results shown in FIG. 14A illustrate that “cocktails” including three MY010 antibodies effectively suppress mast cell responsiveness in all birch-related extracts tested. In particular, cocktails including (in addition to MY010 antibodies 4E1 and 25-3D3) MY010 antibodies 6C9 or 3F1, show similar efficacy compared to the cocktail including (in addition to MY010 antibodies 4E1 and 25-3D3) the MY010 antibody 17B10 belonging to the same complementation group as 6C9 and 3F1. In comparison to the prior art REGN antibody cocktail, the MY010 cocktails of the present invention are more effective in terms of suppression of all birch related allergens tested.

Further, the results shown in FIG. 14B illustrate that the MY010 antibody cocktails of the present invention show superior efficacy on oak compared to the prior art REGN cocktails. In particular, antibody cocktails containing antibodies 6C9 or 3F1, respectively, show even more efficient suppression of mast cell responsiveness in oak extracts than cocktails comprising 17B10 belonging to the same complementation group. These experiments confirm the results of Example 11 represented in FIGS. 11A and 11B.

### Example 15: Efficacy of MY010-3 Cocktail on Birch, Hazel and Oak Pollen Extracts in a Passive Cutaneous Anaphylaxis (PCA) Mouse Model

To study the effects of the MY010-cocktail, an in vivo model of passive cutaneous anaphylaxis was established. Firstly, MY010-cocktail comprising the antibodies 17B10, 25-3D3 and 4E1 was titrated of birch extract. Therefore, huFceRI transgenic mice (IgE/FceRI humanized mouse line, Genoway) were administered subcutaneously with the MY010 antibody cocktail at different doses: 1, 0.1 and 0.01 mg/kg or with 1 mg/Kg of isotype control. Three days later, ears of transgenic mice were intradermally (i.d.) sensitized by injection of pool of human plasma. In the right ear, 20 μL of plasma pool from birch allergic individuals was injected, while the left ear was treated with a plasma pool from healthy individuals (internal experimental control). 24 hours later, animals were challenged by injection with birch extract i.v (0.1 ug) along with Evans blue dye (0.5%). Thirty minutes after challenge, mice were sacrificed and ears were collected to measure ear thickness.

Afterwards, ears were placed in a formamide solution to extract and quantify the dye. Local allergic reaction in the sensitized ears was monitored by the amount of extravasation of the Evans blue staining. The amount of extravasated Evans blue was measured by optical absorption at 620 nm and normalized to the weight of the ear tissue collected (Evans blue ng/mg ear tissue).

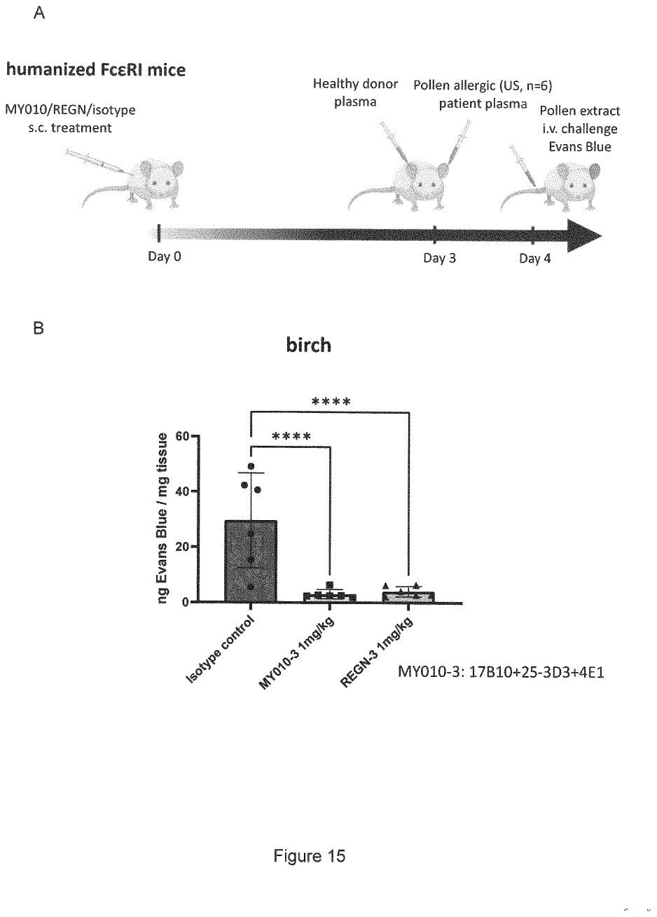

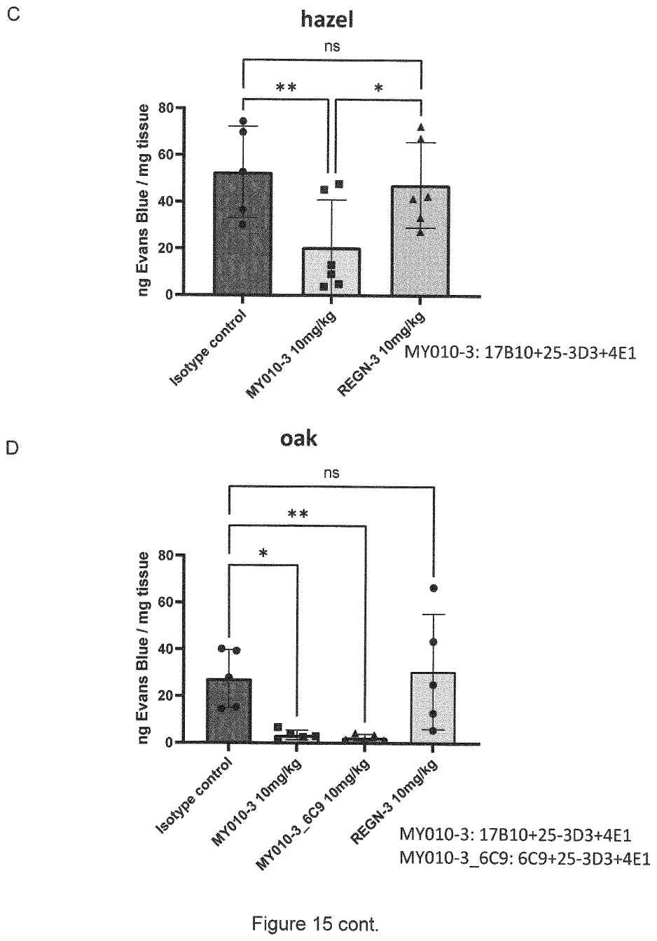

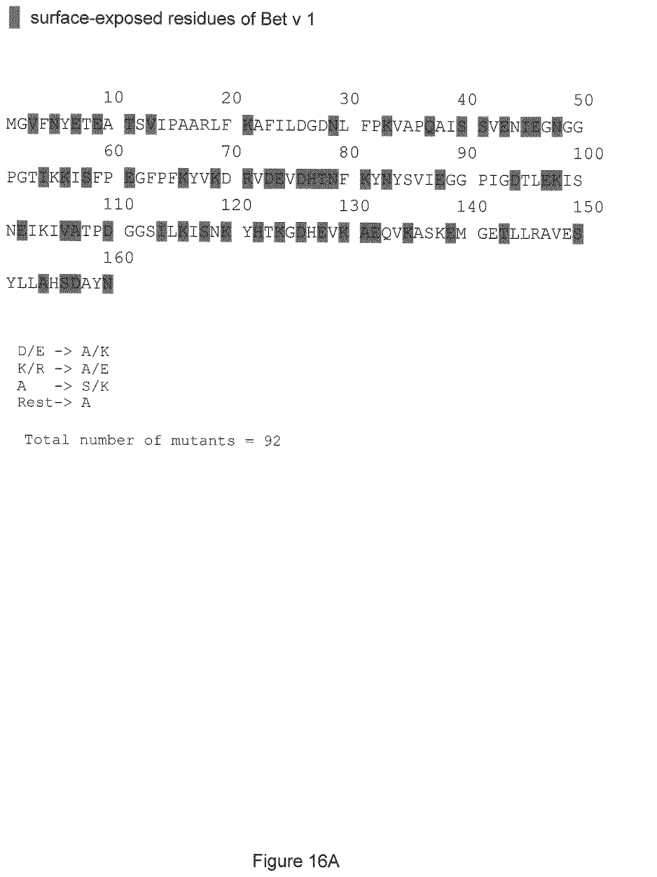

In a second set of experiments, efficacy of MY010 cocktail was compared to REGN cocktail on different PR-10 related extracts. As described above, huFcERI transgenic mice were administered subcutaneously with 0.1 or 1 mg/kg of MY010-cocktail, Regeneron cocktail or isotype control. 3 days later, mice were sensitized intra dermally (i.d.) with plasma derived from healthy donors (left ear) or birch allergic patients (right ear). 24 hours after i.d. sensitization, mice are challenged i.v. with birch pollen related extracts (0.1 ug birch extract, 1 ug alder extract, 1 ug hazel pollen extract and 1 ug oak extract)+evans blue (0.5%). 30 minutes after challenge, mice are sacrificed, and ears are collected to measure ear thickness. Quantification of dye was performed as previously described. The PCA model is schematically shown in FIG. 15A.

The results presented in FIG. 15B show that the MY010-3 cocktail comprising antibodies 17B10, 25-3D3 and 4E1 according to the present invention, and the REGN-3 cocktail comprising prior art antibodies REGN5713, REGN 4715 and REGN 5716 have similar efficacy on birch pollen extract.

However, as shown in FIG. 15C, a MY010-3 cocktail comprising antibodies 17B10, 25-3D3 and 4E1 according to the present invention possesses superior efficacy on hazel pollen extract compared to prior art REGN cocktail.

Moreover, as shown in FIG. 15D, a MY010-3 cocktail comprising antibodies 17B10, 25-3D3 and 4E1, as well as a MY010-3_6C9 cocktail comprising antibodies 6C9, 25-3D3 and 4E1 according to the present invention possess superior efficacy on oak pollen extract compared to REGN cocktail compared to prior art REGN cocktail.

Thus, the superiority of the inventive antibody compositions on birch-related pollen extracts is clearly confirmed also by the in vivo PCA mouse model.

### Example 16: Epitope Mapping

### 1. Production and Purification of Bet v 1 Mutant Library

All surfaced-exposed Bet v 1 residues were mutated according to the following scheme: D/E to A and K; K/R to A and E; A to S and K; Q/N/H/I/L/M/F/S/T/W/YN to A.

The His- and Avi-tagged Bet v 1 mutants were in vivo biotinylated by co-expression of the mutants and the biotinylation enzyme BirA in double-transformed BL21 DE3. The expression was performed in a 96-well plate format. The expression was initiated by inoculating 600 μl of preculture medium (LB with 25 μg/ml Kanamycin, 25 μg/ml Carbenicillin, and 0.5% Glucose) from a glycerol stock or colony into each well of a 96-deepwell plate, which was then incubated overnight at 37° C. On the following day, 1 ml of expression medium (LB with 25 μg/ml Kanamycin, 25 μg/ml Carbenicillin, 1 mM IPTG, and 100 μg/ml Biotin) was added to each well, and the cultures were incubated for 24 hours at room temperature. After expression, the cells were harvested by centrifugation and the supernatant discarded. The recombinant Bet v 1 mutants were then purified via the His-tag. To lyse the cells, 100 μl of BugBuster reagent (Novagen®) was added to each well and incubated for 20 minutes at room temperature. Subsequently, 50 μl of Binding Buffer (50 mM phosphate, 300 mM NaCl, pH 7.4) was added to each well, and the plate was centrifuged for 20 minutes at maximum speed. The supernatant was transferred into a 96 well plate containing magnetic AmMag™ Ni beads (GenScript), which had been prepared according to the manufacturer's instructions. After 30 minutes at room temperature, the magnetic separation rack was used to collect the beads, and the supernatant was discarded. The beads were washed three times by adding 250 μl of wash buffer (50 mM phosphate, 10 mM imidazole, 300 mM NaCl, pH 7.4) to each well. Following thorough mixing, the beads were collected with the magnetic rack, while the supernatant was discarded. Finally, 65 μl of elution buffer (50 mM phosphate, 150 mM imidazole, 300 mM NaCl, pH 7.4) was added and incubated for 5 minutes at room temperature with occasional mixing. The beads were collected using the magnetic rack, and the eluted protein was transferred into a clean 96-well plate.

### 2. Testing Bet v 1 Mutant Library for Epitope Mapping

To test the impact of the individual residues included in the Bet v 1 mutant library on the binding of the MY010 antibodies (4E1, 25-3D3, 17B10, ‘6C9 clonotype’), an ELISA was conducted. Each library variant was tested against all MY010 cocktail antibodies, with two out of the three serving as controls for binding and mutant integrity. High binding, 96-well plates (Greiner 675061) were coated with streptavidin (Sigma, S4762-5MG, 1 μg/ml in PBS, 30 μl/well) for one hour at 37° C. The plates were then washed once with wash buffer (0.05% Tween 20 in PBS, 150 μl/well). The plates were incubated with blocking buffer (2% BSA in PBS, with 100 μl/well) for one hour at room temperature and the blocking solution discarded. The biotinylated Bet v 1 variants were captured for one hour at 37° C. by adding 30 μl/well of the 1:150 diluted mutants in 0.5% BSA in PBS. The plates were then washed four times with wash buffer (150 μl/well). The recombinant Fabs of the MY010 antibodies were added at a concentration of 100 ng/ml, 30 μl/well, and incubated for 2 hours at room temperature. The plates were then washed four times with wash buffer (150 μl/well). Subsequently, 30 μl/well of either mouse anti-human lambda light chain-HRP (Abcam JDC-12, 1:1000 diluted) or goat anti-human kappa light chain-HRP (Invitrogen, 1:5000 diluted) was added and incubated for one hour at room temperature. The plates were then washed four times with wash buffer (150 μl/well). To develop the assay, 30 μl/well of TMB substrate (Sigma T2885, diluted 1:20 in 30 mM citric acid, pH 4.1) was added and incubated for precisely five minutes. The colour development was stopped by the addition of 15 μl/well of H₂SO₄ (AppliChem, A2699, 1 M). The plates were subsequently read at an absorbance of 450 nM without a reference wavelength using a Multiskan FC ELISA reader (Thermo Scientific). The binding of all three MY010 Fabs was analysed for each Bet v 1 mutant. If reduced binding was observed for one of the three Fabs of the MY010 antibodies, compared to the others, the residue mutated of this Bet v 1 variant was designated as part of the binding epitope.

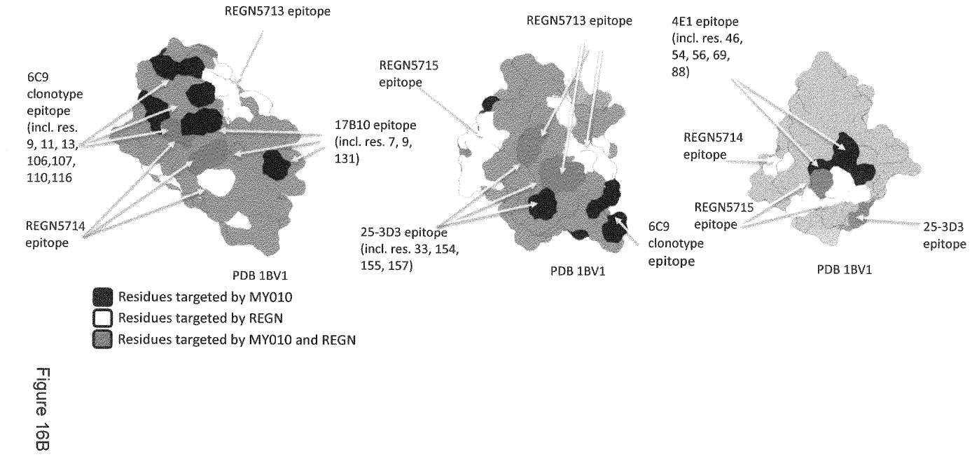

FIG. 16A illustrates the surface-exposed residues of Bet v 1 identified by scanning mutagenesis. The total number of mutants was 92.

The results of epitope mapping by scanning mutagenesis are summarized in Table 9 below.

TABLE 9
Bet v 1 epitope mapping. Epitope residue from Atanasio et al., 2021
(https://www.sciencedirect.com/science/article/pii/S0091674921009039)

| | Bet v 1 | | | | | | | |
|---|---|---|---|---|---|---|---|---|
| # | residue | 6C9 | 17B10 | 4E1 | 25-3D3 | REGN-5713 | REGN-5714 | REGN-5715 |
| 2 | G | | | | | | X | |
| 5 | N | | | | | | X | |
| 7 | E | | X | | | | X | |
| 9 | E | X | X | | | | | |
| 11 | T | X | | | | | | |
| 13 | V | X | | | | | | |
| 33 | K | | | | X | X | | |
| 36 | P | | | | | X | | |
| 37 | Q | | | | | X | | |
| 43 | E | | | | | | | X |
| 44 | N | | | | | | | X |
| 45 | I | | | | | | | X |
| 46 | E | | | X | | | | X |
| 47 | G | | | | | | | X |
| 48 | N | | | | | | | X |
| 49 | G | | | | | | | X |
| 50 | G | | | | | | | X |
| 54 | I | | | X | | | | |
| 56 | K | | | X | | | | |
| 69 | K | | | X | | | | |
| 73 | D | | | | | | X | |
| 74 | E | | | | | | X | |
| 84 | Y | | | | | | X | |
| 88 | E | | | X | | | | |
| 100 | S | | | | | | X | |
| 102 | E | | | | | | X | |
| 106 | V | X | | | | | | |
| 107 | A | X | | | | | | |
| 110 | D | X | | | | | | |
| 116 | K | X | | | | | X | |
| 121 | Y | | | | | | X | |
| 131 | A | | X | | | | | |
| 142 | E | | | | | X | | |
| 146 | R | | | | | X | | |
| 147 | A | | | | | X | | |
| 149 | E | | | | | X | | |
| 150 | S | | | | | X | | |
| 154 | A | | | | X | X | | |
| 155 | H | | | | X | X | | |
| 157 | D | | | | X | | | |

As shown in Table 9 above, MY010 antibodies 6C9, 17B10, 4E1, 25-3D3 are binding to non-overlapping epitopes on Bet v 1. Moreover, it is noted that the epitopes bound by the inventive MY010 antibodies are distinct from the epitopes bound by prior art REGN antibodies.

The above results are schematically illustrated in FIG. 16B.

TABLE OF
SEQUENCES AND SEQ ID NUMBERS (SEQUENCE LISTING):

| | | |
|---|---|---|
| SEQ ID NO | Sequence | Remarks |

Antibody amino acid sequences
4E

| | | |
|---|---|---|
| SEQ ID NO: 1 | TPGVAVG | CDRH1 |
| SEQ ID NO: 2 | LIYWYDEERYSPSLKS | CDRH2 |
| SEQ ID NO: 3 | RPEYNWFDP | CDRH3 |
| SEQ ID NO: 4 | SGSRSNLGENTVT | CDRL1 |
| SEQ ID NO: 5 | RNSQRPS | CDRL2 |
| SEQ ID NO: 6 | AAWDDSLKDWV | CDRL3 |
| SEQ ID NO: 7 | QITLRESGPTLVKPTQTLTLTCNFSGFSLTTPGVA | VH |
| | VGWFRQSPGKAPEWLSLIYWYDEERYSPSLKSRL | |
| | SITKDTSKNQVVLTMTNMDPVDTGTYYCARRPE | |
| | YNWFDPWGQGILVTVSS | |
| SEQ ID NO: 8 | QSVLAQPPSVSETPGQRVTISCSGSRSNLGENTV | VL |
| | TWYQQFPGTAPKLLIYRNSQRPSGVPDRFSASKS | |
| | GTSASLAISGLQSEDEADYYCAAWDDSLKDWVF | |
| | GGGTKLTVL | |

25-3D3

| | | |
|---|---|---|
| SEQ ID NO: 9 | RYWIA | CDRH1 |
| SEQ ID NO: 10 | IMYPGDSDRRYNSSFQG | CDRH2 |
| SEQ ID NO: 11 | MQGYYGPGSYFDY | CDRH3 |
| SEQ ID NO: 12 | QASQDISKHVN | CDRL1 |
| SEQ ID NO: 13 | DASNLKT | CDRL2 |
| SEQ ID NO: 14 | QHYNGLLGWT | CDRL3 |
| SEQ ID NO: 15 | QVQLVQSGAEVKKPGESLRISCKGSGYIFTRYWI | VH |
| | AWVRQLPGKGLEWMGIMYPGDSDRRYNSSFQ | |
| | GHVTFSVDMSINTAYLQWDSLKASDTATYFCAR | |
| | MQGYYGPGSYFDYWGQGTLVTVSS | |
| SEQ ID NO: 16 | AIQLTQSPSSLSASVGDRVAITCQASQDISKHVN | VL |
| | WYQQKPGKAPKLLIYDASNLKTGVPSQFSGSGS | |
| | GTNFTFITNFLQPEDVATYYCQHYNGLLGWTFG | |
| | QGTKVEIK | |

17B10

| | | |
|---|---|---|
| SEQ ID NO: 17 | SYAMS | CDRH1 |
| SEQ ID NO: 18 | TISGSGETTYYADPVKG | CDRH2 |
| SEQ ID NO: 19 | ASSHSSSWFWYFDY | CDRH3 |
| SEQ ID NO: 20 | RASQSVSLNYLA | CDRL1 |
| SEQ ID NO: 21 | GASSRAT | CDRL2 |
| SEQ ID NO: 22 | QQYGNSPPYT | CDRL3 |
| SEQ ID NO: 23 | EVQILESGGGLVQPGGSLRFSCAASGFPFRSYAM | VH |
| | SWVRQAPGKGLEWVSTISGSGETTYYADPVKGR | |
| | FTISRDNSKNTVFLQMNSLGAEDTAIYFCAKASS | |
| | HSSSWFWYFDYWGQGTVVTVSS | |
| SEQ ID NO: 24 | EIVLTQSPGTLSLSLGERVSLSCRASQSVSLNYLA | VL |
| | WYQQKPGQAPRLLIYGASSRATGIPDRFSGSGS | |
| | GTDFTLTISRLEPEDFAVYYCQQYGNSPPYTFGQ | |
| | GTKLEIK | |

Antibody nucleic acid sequences
4E1

| | | |
|---|---|---|
| SEQ ID NO: 25 | ACTCCTGGAGTGGCTGTGGGC | CDRH1 |
| SEQ ID NO: 26 | CTCATTTACTGGTATGATGAAGAGCGCTACA | CDRH2 |
| | GCCCTTCTCTGAAGAGT | |
| SEQ ID NO: 27 | CGACCAGAATACAACTGGTTCGACCCC | CDRH3 |
| SEQ ID NO: 28 | TCAGGAAGCCGGTCCAACCTCGGAGAAAAT | CDRL1 |
| | ACTGTTACC | |
| SEQ ID NO: 29 | CGGAATAGTCAACGGCCCTCA | CDRL2 |
| SEQ ID NO: 30 | GCAGCCTGGGATGACAGCCTGAAGGATTGG | CDRL3 |
| | GTG | |
| SEQ ID NO: 31 | CAGATCACCTTGAGGGAGTCTGGTCCTACGC | VH |
| | TGGTGAAACCCACACAGACCCTCACCCTGAC | |
| | CTGCAACTTCTCTGGGTTCTCACTCACAACTC | |
| | CTGGAGTGGCTGTGGGCTGGTTCCGTCAGT | |
| | CCCCAGGAAAGGCCCCGGAGTGGCTTTCAC | |
| | TCATTTACTGGTATGATGAAGAGCGCTACAG | |
| | CCCTTCTCTGAAGAGTAGACTCTCCATAACCA | |
| | AGGACACCTCCAAAAATCAGGTGGTCCTTAC | |
| | CATGACCAACATGGACCCTGTGGACACAGG | |
| | CACATATTACTGTGCACGCCGACCAGAATAC | |
| | AACTGGTTCGACCCCTGGGGCCAGGGGATC | |
| | CTGGTCACCGTCTCCTCA | |
| SEQ ID NO: 32 | CAGTCTGTGTTGGCTCAGCCACCCTCAGTGT | VL |
| | CTGAGACCCCCGGACAGAGGGTCACCATCT | |
| | CTTGTTCAGGAAGCCGGTCCAACCTCGGAGA | |
| | AAATACTGTTACCTGGTATCAGCAATTCCCAG | |
| | GAACGGCCCCCAAACTCCTCATCTACCGGAA | |
| | TAGTCAACGGCCCTCAGGGGTCCCTGACCG | |
| | ATTTCTGCCTCCAAGTCTGGCACCTCAGCCT | |
| | CCCTGGCCATCAGTGGACTCCAGTCTGAGGA | |
| | TGAGGCTGATTATTATTGTGCAGCCTGGGAT | |
| | GACGCCTGAAGGATTGGGTGTTCGGCGGA | |
| | GGGACCAAGCTGACCGTCCTA | |

25-3D3

| | | |
|---|---|---|
| SEQ ID NO: 33 | AGATACTGGATCGCC | CDRH1 |
| SEQ ID NO: 34 | ATCATGTATCCTGGTGACTCTGATAGAAGAT | CDRH2 |
| | ACAATTCGTCCTTCCAAGGC | |
| SEQ ID NO: 35 | ATGCAGGGTTATTATGGTCCGGGGAGCTACT | CDRH3 |
| | TTGACTAC | |
| SEQ ID NO: 36 | CAGGCGAGTCAGGACATTAGTAAACATGTG | CDRL1 |
| | AAT | |
| SEQ ID NO: 37 | GATGCGTCCAATTTGAAAACA | CDRL2 |
| SEQ ID NO: 38 | CAACACTATAATGGTCTCCTCGGGTGGACG | CDRL3 |
| SEQ ID NO: 39 | CAGGTTCAGCTGGTGCAGTCTGGAGCAGAG | VH |
| | GTGAAAAAGCCCGGGGAGTCTCTTAGGATC | |
| | TCCTGTAAGGGTTCTGGATACATTTTTACCAG | |
| | ATACTGGATCGCCTGGGTGCGCCAGCTGCC | |
| | CGGGAAAGGCCTGGAGTGGATGGGAATCAT | |
| | GTATCCTGGTGACTCTGATAGAAGATACAAT | |
| | TCGTCCTTCCAAGGCCATGTCACCTTCTCAGT | |
| | CGACATGTCCATCAATACCGCCTACCTGCAG | |
| | TGGGACAGCCTGAAGGCCTCGGACACCGCC | |
| | ACTTATTTCTGTGCGAGAATGCAGGGTTATTA | |
| | TGGTCCGGGGAGCTACTTTGACTACTGGGG | |
| | CCAGGGAACCCTGGTCACCGTCTCCTCA | |
| SEQ ID NO: 40 | GCCATCCAGTTGACCCAGTCTCCATCCTCCCT | VL |
| | GTCTGCATCTGTAGGAGACAGAGTCGCCATC | |
| | ACTTGCCAGGCGAGTCAGGACATTAGTAAAC | |
| | ATGTGAATTGGTATCAGCAGAAACCAGGGA | |
| | AAGCCCCAAAACTCCTAATCTACGATGCGTC | |
| | CAATTTGAAAACAGGAGTCCCATCACAGTTC | |
| | AGTGGAAGTGGATCTGGGACAAATTTTACTT | |
| | TCATTACCAACTTCCTGCAGCCTGAAGATGTT | |
| | GCAACATATTACTGTCAACACTATAATGGTCT | |
| | CCTCGGGTGGACGTTCGGCCAAGGGACCAA | |
| | GGTGGAAATCAAA | |

17B10

| | | |
|---|---|---|
| SEQ ID NO: 41 | AGTTATGCCATGAGT | CDRH1 |
| SEQ ID NO: 42 | ACTATTAGTGGTAGTGGTGAAACCACATACT | CDRH2 |
| | ACGCAGACCCCGTGAAGGGC | |
| SEQ ID NO: 43 | GCAAGCTCGCACAGCAGCAGTTGGTTCTGG | CDRH3 |
| | TACTTTGACTAC | |
| SEQ ID NO: 44 | AGGGCCAGTCAGAGTGTTAGCCTCAACTATT | CDRL1 |
| | TAGCC | |
| SEQ ID NO: 45 | GGTGCATCCAGCAGGGCCACT | CDRL2 |
| SEQ ID NO: 46 | CAGCAGTATGGTAACTCACCTCCGTACACT | CDRL3 |
| SEQ ID NO: 47 | GAGGTGCAGATATTGGAGTCTGGGGGAGG | VH |
| | CTTGGTACAGCCTGGGGGGTCCCTGAGATT | |
| | CTCCTGTGCAGCCTCTGGATTCCCCTTTAGG | |
| | AGTTATGCCATGAGTTGGGTCCGCCAGGCTC | |
| | CAGGGAAGGGGCTGGAGTGGGTCTCAACTA | |
| | TTAGTGGTAGTGGTGAAACCACATACTACGC | |
| | AGACCCCGTGAAGGGCCGCTTCACCATCTCA | |
| | AGAGACAATTCCAAGAACACGGTGTTTCTGC | |
| | AAATGAACAGCCTGGGAGCCGAAGACACGG | |
| | CCATATATTTCTGTGCGAAGGCAAGCTCGCA | |
| | CAGCAGCAGTTGGTTCTGGTACTTTGACTAC | |
| | TGGGGCCAGGGAACCGTGGTCACCGTCTCC | |
| | TCA | |
| SEQ ID NO: 48 | GAAATTGTGTTGACGCAGTCTCCAGGCACCC | VL |
| | TGTCTTTGTCTCTAGGGGAAAGAGTTTCCCTC | |
| | TCCTGCAGGGCCAGTCAGAGTGTTAGCCTCA | |
| | ACTATTTAGCCTGGTACCAGCAAAAACCTGG | |
| | CCAGGCTCCCAGGCTCCTCATCTATGGTGCA | |
| | TCCAGCAGGGCCACTGGCATCCCAGACAGA | |
| | TTCAGTGGCAGTGGGTCTGGGACAGACTTC | |
| | ACTCTCACCATCAGCAGACTGGAGCCTGAAG | |
| | ATTTTGCAGTGTATTACTGTCAGCAGTATGGT | |
| | AACTCACCTCCGTACACTTTTGGCCAGGGGA | |
| | CCAAGCTGGAGATCAAA | |

Antibody constant regions

| | | |
|---|---|---|
| SEQ ID NO: 49 | ASTKGPSVFPLAPCSRSTSESTAALGCLVKDYFPEP | human IgG4 (stable |
| | VTVSWNSGALTSGVHTFPAVLQSSGLYSLSSVVT | hinge S228P) |
| | VPSSSLGTKTYTCNVDHKPSNTKVDKRVESKYGP | |
| | PCPPCPAPEFLGGPSVFLFPPKPKDTLMISRTPEVT | |
| | CVVVDVSQEDPEVQFNWYVDGVEVHNAKTKP | |
| | REEQFNSTYRVVSVLTVLHQDWLNGKEYKCKVS | |
| | NKGLPSSIEKTISKAKGQPREPQVYTLPPSQEEMT | |
| | KNQVSLTCLVKGFYPSDIAVEWESNGQPENNYK | |
| | TTPPVLDSDGSFFLYSRLTVDKSRWQEGNVFSCS | |
| | VMHEALHNHYTQKSLSLSLGK* | |
| SEQ ID NO: 50 | RTVAAPSVFIFPPSDEQLKSGTASVVCLLNNFYPR | human kappa LC |
| | EAKVQWKVDNALQSGNSQESVTEQDSKDSTYS | |
| | LSSTLTLSKADYEKHKVYACEVTHQGLSSPVTKSF | |
| | NRGEC* | |
| SEQ ID NO: 51 | GQPKAAPSVTLFPPSSEELQANKATLVCLISDFYP | human lambda LC |
| | GAVTVAWKADSSPVKAGVETTTPSKQSNNKYA | |
| | ASSYLSLTPEQWKSHRSYSCQVTHEGSTVEKTVA | |
| | PTECS* | |

Allergens

| | | |
|---|---|---|
| SEQ ID NO: 52 | MGVFNYETETTSVIPAARLFKAFILDGDNLFPKVA | Bet v 1.0101 |
| | PQAISSVENIEGNGGPGTIKKISFPEGFPFKYVKDR | |
| | VDEVDHTNFKYNYSVIEGGPIGDTLEKISNEIKIV | |
| | ATPDGGSILKISNKYHTKGDHEVKAEQVKASKE | |
| | MGETLLRAVESYLLAHSDAYNASTKGGGGSGLN | |
| | DIFEAQKIEWHEGGGGSHHHHHHHH* | |
| SEQ ID NO: 53 | MGVFNYEVETPSVIPAARLFKSYVLDGDKLIPKVA | Cor a 1.0101 |
| | PQAITSVENVEGNGGPGTIKNITFGEGSRYKYVK | |
| | ERVDEVDNTNFTYSYTVIEGDVLGDKLEKVCHEL | |
| | KIVAAPGGGSILKISSKFHAKGDHEINAEEMKGA | |
| | KEMAEKLLRAVETYLLAHSAEYNASTKGGGGSGL | |
| | NDIFEAQKIEWHEGGGGSHHHHHHH | |
| SEQ ID NO: 54 | MGVFNYETESTSVIPAARLFKAFILDGNNLIPKVA | Cor a 1.0201 |
| | PQAVSSVENVEGNGGPGTIKKITFSEGSPFKYVKE | |
| | RVEEVDHTNFKYSYTVIEGGPVGDKVEKICNEIKI | |
| | VAAPDGGSILKISNKYHTKGDHEVDAEHIKGGK | |
| | EKVEGLFRAVEAYLLAHSDAYNASTKGGGGSGL | |
| | NDIFEAQKIEWHEGGGGSHHHHHHHH* | |
| SEQ ID NO: 55 | MGVFNYEAETPSVIPAARLFKAFILDGDKLLPKVA | Aln g 1.0101 |
| | PEAVSSVENIEGNGGPGTIKKITFPEGSPFKYVKER | |
| | VDEVDRVNFKYSFSVIEGGAVGDALEKVCNEIKI | |
| | VAAPDGGSILKISNKFHTKGDHEINAEQIKIEKEK | |
| | AVGLLKAVESYLLAHSDAYNASTKGGGGSGLN | |
| | DIFEAQKIEWHEGGGGSHHHHHHHH* | |
| SEQ ID NO: 56 | MGVFTYESETTTVITPARLFKAFVLDADNLIPKVA | Fag s 1.0101 |
| | PQAIKSSEIIEGSGGPGTIKKITFGEGSQFNYVKHR | |
| | IDEIDNANFTYACTLIEGDAISETLEKIAYEIKLVAS | |
| | PDGGSILKSTSKYHTKGDHEIKEDQIKAGKEEAS | |
| | GIFKAVEAYLLANPAAYHASTKGGGGSGLNDIFE | |
| | AQKIEWHEGGGGSHHHHHHHH* | |
| SEQ ID NO: 57 | MGVFTHESQETSVIAPARLFKALFLDSDNLIQKVL | Que a 1.0201 |
| | PQAIKSTEIIEGNGGPGTIKKITFGEASKFKYAKHRI | |
| | DALDPENCTYSFSVIEGDALTVMESVSTEIKCVAS | |
| | PDGGSIMKSTTKYQTKGDFQLKEEQIQAAIEKAA | |
| | GLLKAVEAYLVAHPDLYKASTKGGGGSGLNDIF | |
| | EAQKIEWHEGGGGSHHHHHHHH* | |
| SEQ ID NO: 58 | MGVFTHESQETSVIPPARLFKAFVLDSDNLIPKVL | Cas s 1.0101 |
| | PQAIKSTEIIEGNGGPGTIKKITFGEASKYKYSKHRI | |
| | DALDPENCTYSFSVIEGDVLTDIENVSTETKFVAS | |
| | PDGGTIMKSTTKYQTKGDFQLKEEQVQAAIEKA | |
| | TGLFKAVEAYLLANPDLYKASTKGGGGSGLNDIF | |
| | EAQKIEWHEGGGGSHHHHHHHH* | |
| SEQ ID NO: 59 | MGVFNYEAETPSVIPAARLFKSYVLDGDKLIPKVA | Car b 1.1A |
| | PQVISSVENVGGNGGPGTIKNITFAEGIPFKFVKE | |
| | RVDEVDNANFKYNYTVIEGDVLGDKLEKVSHEL | |
| | KIVAAPGGGSIVKISSKFHAKGYHEVNAEKMKGA | |
| | KEMAEKLLRAVESYLLAHTAEYNASTKGGGGSGL | |
| | NDIFEAQKIEWHEGGGGSHHHHHHHH* | |
| SEQ ID NO: 60 | MGVFNYEAETTSVIPAARLFKAFILDGNKLIPKVS | Purchased from |
| | PQAVSSVENVEGNGGPGTIKKITFSEGSPVKYVK | Car b 1.2 |
| | ERVEEIDHTNFKYNYTVIEGDVLGDKLEKVSHEL | Cusabio |
| | KIVAAPGGGSIVKISSKFHAKGYHEVNAEEMKG | |
| | AKEMAEKLLRAVESYLLAHTAEYNASTKGGGGS | |
| | GLNDIFEAQKIEWHEGGGGSHHHHHHHH* | |
| SEQ ID NO: 61 | MGVFNYEAETPSVIPAARLFKSYVLDGDKLIPKVA | Ost c 1.0101 |
| | PQAISKVENVGGNGGPGTIKNITFAEGSPFKFVK | |
| | ERVDEVDNANFKYNYTVIEGDVLGDKLEKVSHE | |
| | LKIVAAPGGGSILKISSKFHAKGDHEVNAEEMKG | |
| | AKEMAEKLLRAVESYLLAHTAEYNASTKGGGGS | |
| | GLNDIFEAQKIEWHEGGGGSHHHHHHHH* | |
| SEQ ID NO: 62 | GVYTFENEFTSEIPPSRLFKAFVLDADNLIPKIAPQ | Mal d 1.0108 |
| | AIKQAEILEGNGGPGTIKKITFGEGSQYGYVKHRI | (purchased from |
| | DSIDEASYSYSYTLIEGDALTDTIEKISYETKLVACG | biomay AG) |
| | SGSTIKSISHYHTKGNIEIKEEHVKAGKEKAHGLFK | |
| | LIESYLKDHPDAYN | |
| SEQ ID NO: 63 | MGVFCYEDEATSVIPPARLFKSFVLDADNLIPKVA | Cor a 1.0401 |
| | PQHFTSAENLEGNGGPGTIKKITFAEGNEFKYMK | (purchased from |
| | HKVEEIDHANFKYCYSIIEGGPLGHTLEKISYEIKM | Indoor |
| | AAAPHGGGSILKITSKYHTKGQASINEEEIKAGKE | Biotechnologies, Inc.) |
| | KAAGLFKAVEAYLLAHPDAYCHHHHHH | |
| SEQ ID NO: 64 | MGVFTYETESTSVIPPARLFKAFVLDADNLIPKVV | Jug r 5.0101 |
| | PQAVKSSEIIEGNGGPGTIKKINFGEGSQYKYVK | (purchased from |
| | HRTDAIDEANFTYAYSVIEGDALADKIEKISYETKI | Indoor |
| | VASHEGGSILKSISHYHSKGDHEIKEEDVKDGKE | Biotechnologies, Inc.) |
| | KASGLFKAVEGYLLAHPDAYN | |
| SEQ ID NO:65 | MGVFNYETETTSVIPAARLFKAFILDGDNLFPKVA | Bet v 1 N160A |
| | PQAISSVENIEGNGGPGTIKKISFPEGFPFKYVKDR | |
| | VDEVDHTNFKYNYSVIEGGPIGDTLEKISNEIKIV | |
| | ATPDGGSILKISNKYHTKGDHEVKAEQVKASKE | |
| | MGETLLRAVESYLLAHSDAYAASTKGGGGSGLN | |
| | DIFEAQKIEWHEGGGGSHHHHHHHH | |

Antibody amino acid sequences
6C9

| | | |
|---|---|---|
| SEQ ID NO: 66 | ADHIH | CDRH1 |
| SEQ ID NO: 67 | WINPYLDGTHYVEKFQD | CDRH2 |
| SEQ ID NO: 68 | KGSDSGWVPYY | CDRH3 |
| SEQ ID NO: 69 | SGITSNIGSNSVN | CDRL1 |
| SEQ ID NO: 70 | SNNHRPS | CDRL2 |
| SEQ ID NO: 71 | TAWDDSLNGPV | CDRL3 |
| SEQ ID NO: 72 | QVQLVESGAEVKRPGASVKVSCKASGYTFIADHI | VH |
| | HWVRQAPGQGLEWMGWINPYLDGTHYVEKF | |
| | QDRVTMTRNTSISTAYMELTRLTFDDTAVYYCA | |
| | KKGSDSGWVPYYWGQGTTVTVSS | |
| SEQ ID NO: 73 | QSVLTQPPSASGTPGQRVTMSCSGITSNIGSNSV | VL |
| | NWYQQLPGTAPKLLIYSNNHRPSGVPDRFSGSK | |
| | SGTSASLAISGLQSEDEADYYCTAWDDSLNGPV | |
| | FGGGTKLTVL | |

3F1

| | | |
|---|---|---|
| SEQ ID NO: 74 | GNHIH | CDRH1 |
| SEQ ID NO: 75 | YINPYTGGTHYAQDFQG | CDRH2 |
| SEQ ID NO: 76 | KASNSGWVPYY | CDRH3 |
| SEQ ID NO: 77 | SGSSSNVGSNSVN | CDRL1 |
| SEQ ID NO: 78 | SNNQRPS | CDRL2 |
| SEQ ID NO: 79 | AAWDDSLIGPV | CDRL3 |
| SEQ ID NO: 80 | QVQLVESGAEVKRPGASVKVSCKASGYTFIADHI | VH |
| | HWVRQAPGQGLEWMGWINPYLDGTHYVEKF | |
| | QDRVTMTRNTSISTAYMELTRLTFDDTAVYYCA | |
| | KKGSDSGWVPYYWGQGTTVTVSS | |
| SEQ ID NO: 81 | QSVLTQPPSASGTPGQRVTMSCSGITSNIGSNSV | VL |
| | NWYQQLPGTAPKLLIYSNNHRPSGVPDRFSGSK | |
| | SGTSASLAISGLQSEDEADYYCTAWDDSLNGPV | |
| | FGGGTKLTVL | |

13A2

| | | |
|---|---|---|
| SEQ ID NO: 82 | ANHIH | CDRH1 |
| SEQ ID NO: 83 | FLNPYTKGTHYPQEFQG | CDRH2 |
| SEQ ID NO: 84 | KDSSSGWVPYY | CDRH3 |
| SEQ ID NO: 85 | SGSGSNVGSNSVN | CDRL1 |
| SEQ ID NO: 86 | SNHQRPS | CDRL2 |
| SEQ ID NO: 87 | AVWDDSLNGPV | CDRL3 |
| SEQ ID NO: 88 | QVQLVESGAEGKKPGASVRVSCTASGYNFIANH | VH |
| | IHWVRQAPGQGLQWLGFLNPYTKGTHYPQEF | |
| | QGRVTMTCDTSISTAYMELTRLRPDDTAVYYCA | |
| | TKDSSSGWVPYYWGQGSTVTVSS | |
| SEQ ID NO: 89 | QSVLTQPPSASGTPGQRVTISCSGSGSNVGSNS | VL |
| | VNWYQQLTGASPKVLIYSNHQRPSGVPDRFSG | |
| | SKSGTSASLAISGLQSEDEAEYYCAVWDDSLNGP | |
| | VFGGGTKLTVL | |

Antibody nucleic acid sequences
6C9

| | | |
|---|---|---|
| SEQ ID NO: 90 | GCCGACCATATCCAC | CDRH1 |
| SEQ ID NO: 91 | TGGATCAACCCTTATCTAGACGGCACACACT | CDRH2 |
| | ATGTAGAGAAGTTTCAGGAC | |
| SEQ ID NO: 92 | AAGGGGTCTGACAGTGGCTGGGTCCCTTAC | CDRH3 |
| | TAC | |
| SEQ ID NO: 93 | TCTGGAATTACCTCCAACATCGGAAGTAACA | CDRL1 |
| | GTGTAAAC | |
| SEQ ID NO: 94 | AGTAACAATCACCGGCCCTCA | CDRL2 |
| SEQ ID NO: 95 | ACAGCATGGGATGACAGCCTGAATGGTCCG | CDRL3 |
| | GTC | |
| SEQ ID NO: 96 | CAGGTGCAGCTGGTGGAGTCCGGGGCTGA | VH |
| | GGTAAAGAGGCCTGGGGCCTCAGTGAAGGT | |
| | GTCCTGCAAGGCTTCTGGATACACCTTCATC | |
| | GCCGACCATATCCACTGGGTGCGACAGGCC | |
| | CCTGGACAAGGGCTTGAGTGGATGGGTTGG | |
| | ATCAACCCTTATCTAGACGGCACACACTATGT | |
| | AGAGAAGTTTCAGGACAGGGTCACCATGAC | |
| | CAGGAACACGTCCATCAGCACAGCCTACATG | |
| | GAGCTGACCAGACTGACCTTTGACGACACG | |
| | GCCGTCTATTACTGTGCGAAAAAGGGGTCTG | |
| | ACAGTGGCTGGGTCCCTTACTACTGGGGCC | |
| | AGGGAACCACGGTCACCGTCTCCTCA | |
| SEQ ID NO: 97 | CAGTCTGTGCTGACGCAGCCACCCTCAGCGT | VL |
| | CTGGGACCCCCGGGCAGAGGGTCACCATGT | |
| | CGTGTTCTGGAATTACCTCCAACATCGGAAG | |
| | TAACAGTGTAAACTGGTACCAGCAACTCCCA | |
| | GGAACGGCCCCCAAACTCCTCATCTATAGTA | |
| | ACAATCACCGGCCCTCAGGGGTCCCTGACC | |
| | GATTCTCTGGCTCCAAGTCTGGCACCTCAGC | |
| | CTCCCTGGCCATCAGTGGACTCCAGTCTGAG | |
| | GATGAGGCTGATTATTACTGTACAGCATGGG | |
| | ATGACAGCCTGAATGGTCCGGTCTTCGGCG | |
| | GAGGGACCAAGCTGACCGTCCTA | |

3F1

| | | |
|---|---|---|
| SEQ ID NO: 98 | GGCAACCACATCCAC | CDRH1 |
| SEQ ID NO: 99 | TACATCAACCCATATACTGGAGGCACACACT | CDRH2 |
| | ATGCACAGGACTTTCAGGGC | |
| SEQ ID NO: 100 | AAGGCCTCTAACAGTGGCTGGGTCCCTTATT | CDRH3 |
| | AC | |
| SEQ ID NO: 101 | TCTGGAAGCAGCTCCAACGTCGGAAGTAATT | CDRL1 |
| | CTGTAAAC | |
| SEQ ID NO: 102 | AGTAATAATCAACGGCCCTCA | CDRL2 |
| SEQ ID NO: 103 | GCAGCCTGGGATGACAGTCTGATTGGTCCG | CDRL3 |
| | GTA | |
| SEQ ID NO: 104 | CAGGTGCAGCTGGTGCAGTCTGGGGCTGAG | VH |
| | GGGAAGAAGCCTGGGGCCTCAGTGAAGGT | |
| | CTCCTGCAAGGCTTCTGGATACACCTTCATTG | |
| | GCAACCACATCCACTGGGTGCGACAGGCCC | |
| | CTGGACAAGGACTTGACTGGATGGGATACA | |
| | TCAACCCATATACTGGAGGCACACACTATGC | |
| | ACAGGACTTTCAGGGCAGGGTCACCATGAC | |
| | CAGCGACACGTCCATCAACACAGCCTACATG | |
| | GAACTGAGCAGGCTGAGACCTGACGACACG | |
| | GCCGTGTATTACTGTGCGACAAAGGCCTCTA | |
| | ACAGTGGCTGGGTCCCTTATTACTGGGGCCA | |
| | GGGATCCCTAGTCACCGTCTCCTCA | |
| SEQ ID NO: 105 | CAGTCTGTGCTGACTCAGCCACCCTCAGTGT | VL |
| | CTGGGACCCCCGGGCAGAGGGTCACCATCT | |
| | CTTGTTCTGGAAGCAGCTCCAACGTCGGAAG | |
| | TAATTCTGTAAACTGGTACCAGCAGCTCACA | |
| | GGAGCGGCCCCCAAAGTATTAATCTACAGTA | |
| | ATAATCAACGGCCCTCAGGGGTCCCTGACC | |
| | GATTCTCTGGCTCCAAGTCTGGCACCTCAGC | |
| | CTCCCTGGCCATCAGTGGGCTCCAGTCTGAG | |
| | GATGAAGCTGACTATTACTGTGCAGCCTGGG | |
| | ATGACAGTCTGATTGGTCCGGTATTCGGCGG | |
| | AGGGACCAAACTGACCGTCCTA | |

13A2

| | | |
|---|---|---|
| SEQ ID NO: 106 | GCCAACCACATACAC | CDRH1 |
| SEQ ID NO: 107 | TTTCTCAACCCATATACTAAAGGCACACACTA | CDRH2 |
| | TCCGCAGGAGTTTCAGGGC | |
| SEQ ID NO: 108 | AAGGACTCTAGCAGTGGCTGGGTCCCTTATT | CDRH3 |
| | AC | |
| SEQ ID NO: 109 | TCTGGAAGCGGCTCCAACGTCGGAAGTAATT | CDRL1 |
| | CTGTTAAC | |
| SEQ ID NO: 110 | AGTAATCATCAGCGGCCCTCA | CDRL2 |
| SEQ ID NO: 111 | GCAGTCTGGGATGACAGTCTCAATGGTCCG | CDRL3 |
| | GTA | |
| SEQ ID NO: 112 | CAGGTGCAGCTGGTGGAGTCTGGGGCTGA | VH |
| | GGGGAAGAAGCCTGGGGCCTCAGTGAGGG | |
| | TCTCCTGCACGGCTTCTGGATATAATTTCATT | |
| | GCCAACCACATACACTGGGTGCGACAGGCC | |
| | CCTGGACAAGGACTTCAGTGGCTGGGATTTC | |
| | TCAACCCATATACTAAAGGCACACACTATCC | |
| | GCAGGAGTTTCAGGGCAGGGTCACCATGAC | |
| | CTGCGACACGTCCATCAGCACAGCCTACATG | |
| | GAACTGACCAGACTGAGACCTGACGACACG | |
| | GCCGTTTATTACTGTGCGACAAAGGACTCTA | |
| | GCAGTGGCTGGGTCCCTTATTACTGGGGCC | |
| | AGGGATCCACGGTCACCGTCTCCTCA | |
| SEQ ID NO: 113 | CAGTCTGTGCTGACGCAGCCACCCTCAGCGT | VL |
| | CTGGGACCCCCGGGCAGAGGGTCACCATCT | |
| | CCTGTTCTGGAAGCGGCTCCAACGTCGGAA | |
| | GTAATTCTGTTAACTGGTACCAACAACTAACA | |
| | GGAGCGTCCCCCAAAGTACTAATCTACAGTA | |
| | ATCATCAGCGGCCCTCAGGGGTCCCTGACC | |
| | GATTCTCTGGTTCCAAGTCTGGCACCTCCGC | |
| | CTCCCTGGCCATCAGTGGCCTCCAGTCTGAG | |
| | GATGAAGCTGAATATTACTGTGCAGTCTGGG | |
| | ATGACAGTCTCAATGGTCCGGTATTCGGCGG | |
| | AGGGACCAAGCTGACCGTCCTA | |

1. An antibody, or an antigen-binding fragment thereof, which specifically binds to Bet v 1 (Betula verrucosa allergen 1).

2. The antibody, or an antigen-binding fragment thereof, according to claim 1, wherein the antibody, or the antigen-binding fragment thereof, further binds specifically to a Bet v 1 homologous allergen.

3. The antibody, or an antigen-binding fragment thereof, according to claim 2, wherein the Bet v 1 homologous allergen is a tree pollen allergen.

4. The antibody, or an antigen-binding fragment thereof, according to claim 3, wherein the Bet v 1 homologous tree pollen allergen is selected from the group consisting of Cor a 1, Aln g 1, Fag s 1, Que a 1, Cas s 1, Ost c 1 and Car b1.

5. The antibody, or an antigen-binding fragment thereof, according to any one of the previous claims, wherein the antibody or the antigen-binding fragment thereof, further binds specifically to a Bet v 1 homologous food allergen.

6. The antibody, or an antigen-binding fragment thereof, according to claim 5, wherein the Bet v 1 homologous food allergen is selected from the group consisting of Mal d 1, Cor a 1 and Jug r 5.

7. The antibody, or an antigen-binding fragment thereof, according to any one of the previous claims, wherein the antibody, or the antigen-binding fragment thereof, comprises a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6.

8. The antibody, or an antigen-binding fragment thereof, according to any one of the previous claims, wherein the antibody, or the antigen-binding fragment thereof, comprises a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6.

9. The antibody, or an antigen-binding fragment thereof, according to any one of the previous claims, wherein the antibody, or the antigen-binding fragment thereof, comprises a VH having at least 70% identity to SEQ ID NO: 7, and a VL having at least 70% identity to SEQ ID NO: 8.

10. The antibody, or an antigen-binding fragment thereof, according to any one of the previous claims, wherein the antibody, or the antigen-binding fragment thereof, comprises a VH according to SEQ ID NO: 7, and a VL according to SEQ ID NO: 8.

11. The antibody, or an antigen-binding fragment thereof, according to any one of claims 1 to 6, wherein the antibody, or the antigen-binding fragment thereof, comprises a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14.

12. The antibody, or an antigen-binding fragment thereof, according to any one of claims 1 to 6 and 11, wherein the antibody, or the antigen-binding fragment thereof, comprises a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14.

13. The antibody, or an antigen-binding fragment thereof, according to any one of claims 1 to 6, 11 and 12, wherein the antibody, or the antigen-binding fragment thereof, comprises a VH having at least 70% identity to SEQ ID NO: 15, and a VL having at least 70% identity to SEQ ID NO: 16.

14. The antibody, or an antigen-binding fragment thereof, according to any one of claims 1 to 6 and 11 to 13, wherein the antibody, or the antigen-binding fragment thereof, comprises a VH according to SEQ ID NO: 15; and a VL according to SEQ ID NO: 16.

15. The antibody, or an antigen-binding fragment thereof, according to any one of claims 1 to 6, wherein the antibody, or the antigen-binding fragment thereof, comprises a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22.

16. The antibody, or an antigen-binding fragment thereof, according to any one of claims 1 to 6 and 15, wherein the antibody, or the antigen-binding fragment thereof, comprises a CDRH1 according to SEQ ID NO: 17, a CDRH2 according to SEQ ID NO: 18, a CDRH3 according to SEQ ID NO: 19, a CDRL1 according to SEQ ID NO: 20, a CDRL2 according to SEQ ID NO: 21, and a CDRL3 according to SEQ ID NO: 22.

17. The antibody, or an antigen-binding fragment thereof, according to any one of claims 1 to 6, 15 and 16, wherein the antibody, or the antigen-binding fragment thereof, comprises a VH having at least 70% identity to SEQ ID NO: 23; and a VL having at least 70% identity to SEQ ID NO: 24.

18. The antibody, or an antigen-binding fragment thereof, according to any one of claims 1 to 6 and 15 to 17, wherein the antibody, or the antigen-binding fragment thereof, comprises a VH according to SEQ ID NO: 23; and a VL according to SEQ ID NO: 24.

19. The antibody, or an antigen-binding fragment thereof, according to any one of claims 1 to 6, wherein the antibody, or the antigen-binding fragment thereof, comprises a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at least 70% identity to SEQ ID NO: 70, and a CDRL3 having at least 70% identity to SEQ ID NO: 71.

20. The antibody, or an antigen-binding fragment thereof, according to any one of claims 1 to 6 and 19, wherein the antibody, or the antigen-binding fragment thereof, comprises a CDRH1 according to SEQ ID NO: 66, a CDRH2 according to SEQ ID NO: 67, a CDRH3 according to SEQ ID NO: 68, a CDRL1 according to SEQ ID NO: 69, a CDRL2 according to SEQ ID NO: 70, and a CDRL3 according to SEQ ID NO: 71.

21. The antibody, or an antigen-binding fragment thereof, according to any one of claims 1 to 6, 19 and 20, wherein the antibody, or the antigen-binding fragment thereof, comprises a VH having at least 70% identity to SEQ ID NO: 72; and a VL having at least 70% identity to SEQ ID NO: 73.

22. The antibody, or an antigen-binding fragment thereof, according to any one of claims 1 to 6 and 19 to 21, wherein the antibody, or the antigen-binding fragment thereof, comprises a VH according to SEQ ID NO: 72; and a VL according to SEQ ID NO: 73.

23. The antibody, or an antigen-binding fragment thereof, according to any one of claims 1 to 6, wherein the antibody, or the antigen-binding fragment thereof, comprises a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79.

24. The antibody, or an antigen-binding fragment thereof, according to any one of claims 1 to 6 and 23, wherein the antibody, or the antigen-binding fragment thereof, comprises a CDRH1 according to SEQ ID NO: 74, a CDRH2 according to SEQ ID NO: 75, a CDRH3 according to SEQ ID NO: 76, a CDRL1 according to SEQ ID NO: 77, a CDRL2 according to SEQ ID NO: 78, and a CDRL3 according to SEQ ID NO: 79.

25. The antibody, or an antigen-binding fragment thereof, according to any one of claims 1 to 6, 23 and 24, wherein the antibody, or the antigen-binding fragment thereof, comprises a VH having at least 70% identity to SEQ ID NO: 80; and a VL having at least 70% identity to SEQ ID NO: 81.

26. The antibody, or an antigen-binding fragment thereof, according to any one of claims 1 to 6 and 23 to 25, wherein the antibody, or the antigen-binding fragment thereof, comprises a VH according to SEQ ID NO: 80; and a VL according to SEQ ID NO: 81.

27. The antibody, or an antigen-binding fragment thereof, according to any one of claims 1 to 6, wherein the antibody, or the antigen-binding fragment thereof, comprises a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87.

28. The antibody, or an antigen-binding fragment thereof, according to any one of claims 1 to 6 and 27, wherein the antibody, or the antigen-binding fragment thereof, comprises a CDRH1 according to SEQ ID NO: 82, a CDRH2 according to SEQ ID NO: 83, a CDRH3 according to SEQ ID NO: 84, a CDRL1 according to SEQ ID NO: 85, a CDRL2 according to SEQ ID NO: 86, and a CDRL3 according to SEQ ID NO: 87.

29. The antibody, or an antigen-binding fragment thereof, according to any one of claims 1 to 6, 27 and 28, wherein the antibody, or the antigen-binding fragment thereof, comprises a VH having at least 70% identity to SEQ ID NO: 88; and a VL having at least 70% identity to SEQ ID NO: 89.

30. The antibody, or an antigen-binding fragment thereof, according to any one of claims 1 to 6 and 27 to 29, wherein the antibody, or the antigen-binding fragment thereof, comprises a VH according to SEQ ID NO: 88; and a VL according to SEQ ID NO: 89.

31. The antibody, or an antigen-binding fragment thereof, according to any one of the previous claims, wherein the CDRs or the variable regions of the antibody, or the antigen-binding fragment thereof, are human or are derived from human CDR or variable region sequences.

32. The antibody, or an antigen-binding fragment thereof, according to any one of the previous claims, wherein the antibody, or the antigen-binding fragment thereof, is a human antibody.

33. The antibody, or an antigen-binding fragment thereof, according to any one of the previous claims, wherein the antibody, or the antigen-binding fragment thereof, is a monoclonal antibody.

34. The antibody according to any one of the previous claims, wherein the antibody comprises an Fc moiety.

35. The antibody according to any one of the previous claims, wherein the antibody is of the IgG or IgA type.

36. The antibody according to claim 23, wherein the antibody is of the IgG1 or IgG4 type, preferably of the IgG4 type.

37. The antibody according to any one according to the previous claims, wherein the variable regions or the CDRs are derived from an IgE antibody and grafted in a scaffold of an IgG or IgA antibody.

38. The antibody, or an antigen-binding fragment thereof, according to any one of the previous claims, wherein the antibody, or the antigen-binding fragment thereof, is purified.

39. The antibody, or an antigen-binding fragment thereof, according to any one of the previous claims, wherein the antibody, or the antigen-binding fragment thereof, is a single-chain antibody.

40. The antibody, or an antigen-binding fragment thereof, according to claim 20, wherein the antibody, or the antigen-binding fragment thereof, is an scFv.

41. The antibody, or an antigen-binding fragment thereof, according to any one of claims 1 to 19, wherein the antibody, or the antigen-binding fragment thereof, is selected from Fab, Fab′, F(ab′)2 and Fv.

42. The antibody, or an antigen-binding fragment thereof, according to any one of the previous claims, wherein the antibody, or the antigen-binding fragment thereof, is a multispecific antibody or a multispecific antigen-binding fragment.

43. The antibody, or an antigen-binding fragment thereof, according to claim 42, wherein the multispecific antibody, or the multispecific antigen-binding fragment thereof, binds to distinct, non-overlapping epitopes of Bet v 1, and optionally binds to distinct, non-overlapping epitopes of at least one bet v 1 homologous allergen.

44. The antibody, or an antigen-binding fragment thereof, according to claim 43, wherein the multispecific antibody, or the multispecific antigen-binding fragment thereof comprises at least two of the following:
(i) an antigen-binding site comprising (a) the CDRH1, CDRH2, CDRH3, CDRL1, CDRL2 and CDRL3 sequences and/or (b) the VH and VL sequences as defined in any one of claims 7 to 10;
(ii) an antigen-binding site comprising (a) the CDRH1, CDRH2, CDRH3, CDRL1, CDRL2 and CDRL3 sequences and/or (b) the VH and VL sequences as defined in any one of claims 11 to 14; and an antigen-binding site selected from any one of (iii) to (vi):
(iii) an antigen-binding site comprising (a) the CDRH1, CDRH2, CDRH3, CDRL1, CDRL2 and CDRL3 sequences and/or (b) the VH and VL sequences as defined in any one of claims 15 to 18;
(iv) an antigen-binding site comprising (a) the CDRH1, CDRH2, CDRH3, CDRL1, CDRL2 and CDRL3 sequences and/or (b) the VH and VL sequences as defined in any one of claims 19 to 22;
(v) an antigen-binding site comprising (a) the CDRH1, CDRH2, CDRH3, CDRL1, CDRL2 and CDRL3 sequences and/or (b) the VH and VL sequences as defined in any one of claims 23 to 26;
(vi) an antigen-binding site comprising (a) the CDRH1, CDRH2, CDRH3, CDRL1, CDRL2 and CDRL3 sequences and/or (b) the VH and VL sequences as defined in any one of claims 27 to 30.

45. The antibody or the antigen-binding fragment according to any one of the claims 7, 8, 11, 12, 15, 16, 19, 20, 23, 24, 27 or 28, wherein the CDRH1, CDRH2 and CDRH3 sequences are contained by a human variable heavy chain framework region VH chain selected from the group consisting of the amino acid sequences encoded by the genes IGHV1-18, IGHV1-2, IGHV1-24, IGHV1-3, IGHV1-45, IGHV1-46, IGHV1-58, IGHV1-69, IGHV1-69-2, IGHV1-69D, IGHV1-8, IGHV2-26, IGHV2-5, IGHV2-70, IGHV2-70D, IGHV3-11, IGHV3-13, IGHV3-15, IGHV3-20, IGHV3-21, IGHV3-23, IGHV3-23D, IGHV3-30, IGHV3-30-3, IGHV3-30-5, IGHV3-33, IGHV3-35, IGHV3-43, IGHV3-43D, IGHV3-48, IGHV3-49, IGHV3-53, IGHV3-62, IGHV3-64, IGHV3-64D, IGHV3-66, IGHV3-7, IGHV3-72, IGHV3-73, IGHV3-74, IGHV3-9, IGHV3-NL1, IGHV4-28, IGHV4-30-1, IGHV4-30-2, IGHV4-30-4, IGHV4-31, IGHV4-34, and IGHV4-38-2.

46. The antibody or the antigen-binding fragment according to any one of the claims 7, 8, 11, 12, 15, 16, 19, 20, 23, 24, 27, 28 or 45, wherein the CDRL1, CDRL2 and CDRL3 sequences are contained by a human variable light chain framework region VL chain selected from the group consisting of the amino acid sequences encoded by the genes IGLV1-36, IGLV1-40, IGLV1-44, IGLV1-47, IGLV1-51, IGLV10-54, IGLV2-11, IGLV2-14, IGLV2-18, IGLV2-23, IGLV2-8, IGLV3-1, IGLV3-10, IGLV3-12, IGLV3-16, IGLV3-19, IGLV3-21, IGLV3-22, IGLV3-25, IGLV3-27, IGLV3-9, IGLV4-3, IGLV4-60, IGLV4-69, IGLV5-37, IGLV5-39, IGLV5-45, IGLV5-52, IGLV6-57, IGLV7-43, IGLV7-46, IGLV8-61, and IGLV9-49.

47. The antibody or the antigen-binding fragment according to any one of the claims 7, 8, 11, 12, 15, 16, 19, 20, 23, 24, 27, 28 or 45, wherein the CDRL1, CDRL2 and CDRL3 sequences are contained by a human variable light chain framework region VK chain selected from the group consisting of the amino acid sequences encoded by the genes IGKV1-12, IGKV1-13, IGKV1-16, IGKV1-17, IGKV1-27, IGKV1-33, IGKV1-39, IGKV1-5, IGKV1-6, IGKV1-8, IGKV1-9, IGKV1-NL1, IGKV1D-12, IGKV1D-13, IGKV1D-16, IGKV1D-17, IGKV1D-33, IGKV1D-39, IGKV1D-43, IGKV1D-8, IGKV2-24, IGKV2-28, IGKV2-29, IGKV2-30, IGKV2-40, IGKV2D-26, IGKV2D-28, IGKV2D-29, IGKV2D-30, IGKV2D-40, IGKV3-11, IGKV3-15, IGKV3-20, IGKV3D-11, IGKV3D-15, IGKV3D-20, IGKV3D-7, IGKV4-1, IGKV5-2, IGKV6-21, and IGKV6D-21.

48. The antibody, or an antigen-binding fragment thereof, according to any one of the previous claims for use as a medicament.

49. The antibody, or an antigen-binding fragment thereof, for use according to claim 48 in prophylaxis or treatment of birch pollen allergy, and/or related tree pollen allergies, and/or related food allergies.

50. A nucleic acid molecule comprising a polynucleotide encoding the antibody, or an antigen-binding fragment thereof, according to any one of claims 1 to 47.

51. The nucleic acid molecule according to claim 50 comprising a nucleic acid sequence as set forth in any one of SEQ ID NOs 25 to 48, and 90 to 111; or a sequence variant thereof having at least 50%, at least 55%, at least 60%, at least 65%, at least 70%, at least 75%, at least 80%, at least 85%, at least 88%, at least 90%, at least 92%, at least 95%, at least 96%, at least 97%, at least 98% or at least 99% sequence identity.

52. A plurality of nucleic acid molecules encoding the antibody, or an antigen-binding fragment thereof, according to any one of claims 1 to 49, wherein each of the nucleic acid molecules comprises a polynucleotide encoding an immunoglobulin chain of the antibody, or an antigen-binding fragment thereof.

53. The combination of nucleic acid molecules according to claim 52 comprising a nucleic acid sequence as set forth in any one of SEQ ID NOs 25 to 48, and 90 to 111; or a sequence variant thereof having at least 50%, at least 55%, at least 60%, at least 65%, at least 70%, at least 75%, at least 80%, at least 85%, at least 88%, at least 90%, at least 92%, at least 95%, at least 96%, at least 97%, at least 98% or at least 99% sequence identity.

54. A vector comprising the nucleic acid molecule according to claim 50 or 51 or the plurality of nucleic acid molecules according to claim 52 or 53.

55. A plurality of vectors comprising the plurality of nucleic acid molecules according to claim 52 or 53.

56. A host cell expressing the antibody, or an antigen-binding fragment thereof, according to any one of claims 1 to 49, or comprising the vector according to claim 54 or the plurality of vectors of claim 55.

57. A method for preparing the antibody, or an antigen-binding fragment thereof, according any one of claims 1 to 49, or immunoglobulin chain(s) thereof, said method comprising
(i) culturing the host cell according to claim 56; and
(ii) isolating the antibody or immunoglobulin chain(s) thereof from the culture.

58. A composition comprising the antibody, or an antigen-binding fragment thereof, according to any one of claims 1 to 49, the nucleic acid or the plurality of nucleic acids according to any one of claims 50 to 53, the vector or the plurality of vectors according to claim 54 or 55, or the cell according to claim 56.

59. The composition according to claim 58 further comprising a pharmaceutically acceptable excipient, diluent or carrier.

60. The composition according to claim 58 or 59, wherein the composition comprises at least two distinct antibodies, or antigen-binding fragments thereof, binding to distinct, non-overlapping epitopes of Bet v 1, and optionally binding to distinct, non-overlapping epitopes of at least one Bet v 1 homologous protein.

61. The composition according to claim 60, wherein the composition comprises at least two antibodies or antigen-binding fragments thereof, selected from the group consisting of:
(i) an antibody, or an antigen-binding fragment thereof, according to any one of claims 7 to 10;
(ii) an antibody, or an antigen-binding fragment thereof, according to any one of claims 11 to 14; and an antibody, or an antigen-binding fragment thereof, according to any one of (iii) to (vi):
(iii) an antibody, or an antigen-binding fragment thereof, according to any one of claims 15 to 18;
(iv) an antibody, or an antigen-binding fragment thereof, according to any one of claims 19 to 22;
(v) an antibody, or an antigen-binding fragment thereof, according to any one of claims 23 to 26;
(vi) an antibody, or an antigen-binding fragment thereof, according to any one of claims 27 to 30.

62. The composition according to claim 60 or 61, which comprises at least three distinct antibodies, or antigen-binding fragments thereof, and preferably comprises an antibody, or an antigen-binding fragment thereof, according to (i), an antibody, or an antigen-binding fragment thereof, according to (ii), and an antibody, or an antigen-binding fragment thereof, according to at least one of (iii) to (vi), as defined in claim 61.

63. A composition comprising at least two distinct antibodies, or antigen-binding fragments thereof, wherein the two distinct antibodies or antigen-binding fragments bind to distinct, non-overlapping epitopes of Bet v 1, and optionally bind to distinct, non-overlapping epitopes of at least one Bet v 1 homologous protein, wherein the composition comprises at least two antibodies, or antigen-binding fragments thereof, selected from the group consisting of:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14; and an antibody, or an antigen-binding fragment thereof according to any one of (iii) to (vi):
(iii) an antibody, or an antigen-binding fragment thereof, comprising a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antibody, or an antigen-binding fragment thereof, comprising a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at least 70% identity to SEQ ID NO: 70, and a CDRL3 having at least 70% identity to SEQ ID NO: 71;
(v) an antibody, or an antigen-binding fragment thereof, comprising a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79;
(vi) an antibody, or an antigen-binding fragment thereof, comprising a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87.

64. The composition according to claim 63, wherein the composition comprises at least two antibodies or antigen-binding fragments thereof, selected from the group consisting of:
(i) an antibody, or an antigen-binding fragment thereof, comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14; and an antibody, or an antigen-binding fragment thereof according to any one of (iii) to (vi):
(iii) an antibody, or an antigen-binding fragment thereof, comprising a CDRH1 according to SEQ ID NO: 17, a CDRH2 according to SEQ ID NO: 18, a CDRH3 according to SEQ ID NO: 19, a CDRL1 according to SEQ ID NO: 20, a CDRL2 according to SEQ ID NO: 21, and a CDRL3 according to SEQ ID NO: 22;
(iv) an antibody, or an antigen-binding fragment thereof, comprising a CDRH1 according to SEQ ID NO: 66, a CDRH2 according to SEQ ID NO: 67, a CDRH3 according to SEQ ID NO: 68, a CDRL1 according to SEQ ID NO: 69, a CDRL2 according to SEQ ID NO: 70, and a CDRL3 according to SEQ ID NO: 71;
(v) an antibody, or an antigen-binding fragment thereof, comprising a CDRH1 according to SEQ ID NO: 74, a CDRH2 according to SEQ ID NO: 75, a CDRH3 according to SEQ ID NO: 76, a CDRL1 according to SEQ ID NO: 77, a CDRL2 according to SEQ ID NO: 78, and a CDRL3 according to SEQ ID NO: 79;
(vi) an antibody, or an antigen-binding fragment thereof, comprising a CDRH1 according to SEQ ID NO: 82, a CDRH2 according to SEQ ID NO: 83, a CDRH3 according to SEQ ID NO: 84, a CDRL1 according to SEQ ID NO: 85, a CDRL2 according to SEQ ID NO:86, and a CDRL3 according to SEQ ID NO: 87.

65. The composition according to claim 63 or 64, wherein the composition comprises at least two antibodies or antigen-binding fragments thereof, selected from the group consisting of:
(i) an antibody, or an antigen-binding fragment thereof, comprising a VH having at least 70% identity to SEQ ID NO: 7; and a VL having at least 70% identity to SEQ ID NO: 8;
(ii) an antibody, or an antigen-binding fragment thereof, comprising a VH having at least 70% identity to SEQ ID NO: 15; and a VL having at least 70% identity to SEQ ID NO: 16; and an antibody according to any one of (iii) to (vi):
(iii) an antibody, or an antigen-binding fragment thereof, comprising a VH having at least 70% identity to SEQ ID NO: 23; and a VL having at least 70% identity to SEQ ID NO: 24;
(iv) an antibody, or an antigen-binding fragment thereof, comprising a VH having at least 70% identity to SEQ ID NO: 72; and a VL having at least 70% identity to SEQ ID NO: 73;
(v) an antibody, or an antigen-binding fragment thereof, comprising a VH having at least 70% identity to SEQ ID NO: 80; and a VL having at least 70% identity to SEQ ID NO: 81;
(vi) an antibody, or an antigen-binding fragment thereof, comprising a VH having at least 70% identity to SEQ ID NO: 88; and a VL having at least 70% identity to SEQ ID NO: 89.

66. The composition according to any one of claims 63 to 65, wherein the composition comprises at least two antibodies or antigen-binding fragments thereof, selected from the group consisting of:
(i) an antibody, or an antigen-binding fragment thereof, comprising a VH according to SEQ ID NO: 7; and a VL according to SEQ ID NO: 8;
(ii) an antibody, or an antigen-binding fragment thereof, comprising a VH according to SEQ ID NO: 15; and a VL according to SEQ ID NO: 16; and an antibody, or an antigen-binding fragment thereof, according to any one of (iii) to (vi):
(iii) an antibody, or an antigen-binding fragment thereof, comprising a VH according to SEQ ID NO: 23; and a VL according to SEQ ID NO: 24;
(iv) an antibody, or an antigen-binding fragment thereof, comprising a VH according to SEQ ID NO: 72; and a VL according to SEQ ID NO: 73;
(v) an antibody, or an antigen-binding fragment thereof, comprising a VH according to SEQ ID NO: 80; and a VL according to SEQ ID NO: 81;
(vi) an antibody, or an antigen-binding fragment thereof, comprising a VH according to SEQ ID NO: 88; and a VL according to SEQ ID NO: 89.

67. The composition according to any one of claims 63 to 66, which comprises at least three distinct antibodies, or antigen-binding fragments thereof, and preferably comprises an antibody, or an antigen-binding fragment thereof according to (i), an antibody, or an antigen-binding fragment thereof, according to (ii), and an antibody, or an antigen-binding fragment thereof, according to at least one of (iii) to (vi).

68. The composition according to any one of claims 58 to 67, wherein the composition further comprises at least one additional agent useful for treating birch pollen allergy and/or a related tree pollen allergy, and/or a related food allergy.

69. The composition according to claim 68, wherein the additional agent useful for treating birch pollen allergy and/or a related allergy is selected from the group consisting of: 0-adrenergic agonists, epinephrine, antihistamine, corticosteroid, anti-IgE antibody, anti-IgE antibody binding fragment, peptide vaccine and further antibodies capable of binding to a birch pollen allergen.

70. The composition according to any one of claims 58 to 69, wherein the composition further comprises a birch pollen allergen and/or a Bet v 1 homologous allergen, preferably selected from the group consisting of Cor a 1, Ain g 1, Fag s 1, Que a 1, Cas s 1, Ost c 1, Car b1, Mal d 1, Jug r 5 and Pru p 1, and/or comprises an extract comprising a birch pollen allergen and/or a Bet v 1 homologous allergen, preferably selected from the group consisting of Cor a 1, Ain g 1, Fag s 1, Que a 1, Cas s 1, Ost c 1, Car b1, Mal d 1, Jug r 5 and Pru p 1.

71. A kit comprising one or more of
(i) the antibody, or an antigen-binding fragment thereof, according to any one of claims 1 to 49;
(ii) the nucleic acid molecule(s) according to any one of claims 50 to 53;
(iii) the vector(s) according to claim 54 or 55;
(iv) the cell according to claim 56; and/or
(v) the composition according to any one of claims 59 to 70.

72. The kit according to claim 71, wherein the kit comprises
(i) at least two antibodies, or antigen-binding fragments thereof, according to any one of claims 1 to 49, which bind to distinct, non-overlapping epitopes of Bet v 1, and optionally bind to distinct, non-overlapping epitopes of at least one Bet v 1 homologous protein;
(ii) nucleic acid molecule(s) encoding at least two antibodies, or antigen-binding fragments thereof, according to any one of claims 1 to 49, which bind to distinct, non-overlapping epitopes of Bet v 1, and optionally bind to distinct, non-overlapping epitopes of at least one Bet v 1 homologous protein; or
(iii) composition(s) comprising (i) or (ii).

73. The kit according to claim 72 comprising three antibodies, or antigen-binding fragments thereof, according to any one of claims 1 to 49, which bind to distinct, non-overlapping epitopes of Bet v 1, and optionally bind to distinct, non-overlapping epitopes of at least one bet v 1 homologous protein; or nucleic acid(s) encoding said antibodies or compositions comprising said antibodies.

74. A kit comprising at least two antibodies, or antigen-binding fragments thereof, wherein the three antibodies or antigen-binding fragments bind to distinct, non-overlapping epitopes of Bet v 1 and optionally bind to distinct, non-overlapping epitopes of at least one bet v 1 homologous protein, wherein the kit comprises at least two antibodies or antigen-binding fragments thereof, selected from the group consisting of:
(i) an antibody, or an antigen-binding fragment thereof, comprising a CDRH1 having at least 70% identity to SEQ ID NO: 1, a CDRH2 having at least 70% identity to SEQ ID NO: 2, a CDRH3 having at least 70% identity to SEQ ID NO: 3, a CDRL1 having at least 70% identity to SEQ ID NO: 4, a CDRL2 having at least 70% identity to SEQ ID NO: 5, and a CDRL3 having at least 70% identity to SEQ ID NO: 6;
(ii) an antibody, or an antigen-binding fragment thereof, comprising a CDRH1 having at least 70% identity to SEQ ID NO: 9, a CDRH2 having at least 70% identity to SEQ ID NO: 10, a CDRH3 having at least 70% identity to SEQ ID NO: 11, a CDRL1 having at least 70% identity to SEQ ID NO: 12, a CDRL2 having at least 70% identity to SEQ ID NO: 13, and a CDRL3 having at least 70% identity to SEQ ID NO: 14; and an antibody, or an antigen-binding fragment thereof, according to any one of (iii) to (vi):
(iii) an antibody, or an antigen-binding fragment thereof, comprising a CDRH1 having at least 70% identity to SEQ ID NO: 17, a CDRH2 having at least 70% identity to SEQ ID NO: 18, a CDRH3 having at least 70% identity to SEQ ID NO: 19, a CDRL1 having at least 70% identity to SEQ ID NO: 20, a CDRL2 having at least 70% identity to SEQ ID NO: 21, and a CDRL3 having at least 70% identity to SEQ ID NO: 22;
(iv) an antibody, or an antigen-binding fragment thereof, comprising a CDRH1 having at least 70% identity to SEQ ID NO: 66, a CDRH2 having at least 70% identity to SEQ ID NO: 67, a CDRH3 having at least 70% identity to SEQ ID NO: 68, a CDRL1 having at least 70% identity to SEQ ID NO: 69, a CDRL2 having at least 70% identity to SEQ ID NO: 70, and a CDRL3 having at least 70% identity to SEQ ID NO: 71;
(v) an antibody, or an antigen-binding fragment thereof, comprising a CDRH1 having at least 70% identity to SEQ ID NO: 74, a CDRH2 having at least 70% identity to SEQ ID NO: 75, a CDRH3 having at least 70% identity to SEQ ID NO: 76, a CDRL1 having at least 70% identity to SEQ ID NO: 77, a CDRL2 having at least 70% identity to SEQ ID NO: 78, and a CDRL3 having at least 70% identity to SEQ ID NO: 79;
(vi) an antibody, or an antigen-binding fragment thereof, comprising a CDRH1 having at least 70% identity to SEQ ID NO: 82, a CDRH2 having at least 70% identity to SEQ ID NO: 83, a CDRH3 having at least 70% identity to SEQ ID NO: 84, a CDRL1 having at least 70% identity to SEQ ID NO: 85, a CDRL2 having at least 70% identity to SEQ ID NO: 86, and a CDRL3 having at least 70% identity to SEQ ID NO: 87.

75. The kit according to claim 74, wherein the kit comprises at least two antibodies or antigen-binding fragments thereof, selected from the group consisting of:
(i) an antibody, or an antigen-binding fragment thereof, comprising a CDRH1 according to SEQ ID NO: 1, a CDRH2 according to SEQ ID NO: 2, a CDRH3 according to SEQ ID NO: 3, a CDRL1 according to SEQ ID NO: 4, a CDRL2 according to SEQ ID NO: 5, and a CDRL3 according to SEQ ID NO: 6;
(ii) an antibody, or an antigen-binding fragment thereof, comprising a CDRH1 according to SEQ ID NO: 9, a CDRH2 according to SEQ ID NO: 10, a CDRH3 according to SEQ ID NO: 11, a CDRL1 according to SEQ ID NO: 12, a CDRL2 according to SEQ ID NO: 13, and a CDRL3 according to SEQ ID NO: 14; and an antibody, or an antigen-binding fragment thereof, according to any one of (iii) to (vi):
(iii) an antibody, or an antigen-binding fragment thereof, comprising a CDRH1 according to SEQ ID NO: 17, a CDRH2 according to SEQ ID NO: 18, a CDRH3 according to SEQ ID NO: 19, a CDRL1 according to SEQ ID NO: 20, a CDRL2 according to SEQ ID NO: 21, and a CDRL3 according to SEQ ID NO: 22;
(iv) an antibody, or an antigen-binding fragment thereof, comprising a CDRH1 according to SEQ ID NO: 66, a CDRH2 according to SEQ ID NO: 67, a CDRH3 according to SEQ ID NO: 68, a CDRL1 according to SEQ ID NO: 69, a CDRL2 according to SEQ ID NO: 70, and a CDRL3 according to SEQ ID NO: 71;
(v) an antibody, or an antigen-binding fragment thereof, comprising a CDRH1 according to SEQ ID NO: 74, a CDRH2 according to SEQ ID NO: 75, a CDRH3 according to SEQ ID NO: 76, a CDRL1 according to SEQ ID NO: 77, a CDRL2 according to SEQ ID NO: 78, and a CDRL3 according to SEQ ID NO: 79;
(vi) an antibody, or an antigen-binding fragment thereof, comprising a CDRH1 according to SEQ ID NO: 82, a CDRH2 according to SEQ ID NO: 83, a CDRH3 according to SEQ ID NO: 84, a CDRL1 according to SEQ ID NO: 85, a CDRL2 according to SEQ ID NO:86, and a CDRL3 according to SEQ ID NO: 87.

76. The kit according to claim 74 or 75, wherein the kit comprises at least two antibodies or antigen-binding fragments thereof, selected from the group consisting of:
(i) an antibody, or an antigen-binding fragment thereof, comprising a VH having at least 70% identity to SEQ ID NO: 7; and a VL having at least 70% identity to SEQ ID NO: 8;
(ii) an antibody, or an antigen-binding fragment thereof, comprising a VH having at least 70% identity to SEQ ID NO: 15; and a VL having at least 70% identity to SEQ ID NO: 16; and an antibody, or an antigen-binding fragment thereof, according to any one of (iii) to (vi):
(iii) an antibody, or an antigen-binding fragment thereof, comprising a VH having at least 70% identity to SEQ ID NO: 23; and a VL having at least 70% identity to SEQ ID NO: 24;
(iv) an antibody, or an antigen-binding fragment thereof, comprising a VH having at least 70% identity to SEQ ID NO: 72; and a VL having at least 70% identity to SEQ ID NO: 73;
(v) an antibody, or an antigen-binding fragment thereof, comprising a VH having at least 70% identity to SEQ ID NO: 80; and a VL having at least 70% identity to SEQ ID NO: 81;
(vi) an antibody, or an antigen-binding fragment thereof, comprising a VH having at least 70% identity to SEQ ID NO: 88; and a VL having at least 70% identity to SEQ ID NO: 89.

77. The kit according to any one of claims 74 to 76, wherein the kit comprises at least two antibodies or antigen-binding fragments thereof, selected from the group consisting of:
(i) an antibody, or an antigen-binding fragment thereof, comprising a VH according to SEQ ID NO: 7; and a VL according to SEQ ID NO: 8;
(ii) an antibody, or an antigen-binding fragment thereof, comprising a VH according to SEQ ID NO: 15; and a VL according to SEQ ID NO: 16; and an antibody, or an antigen-binding fragment thereof, according to any one of (iii) to (vi):
(iii) an antibody, or an antigen-binding fragment thereof, comprising a VH according to SEQ ID NO: 23; and a VL according to SEQ ID NO: 24;
(iv) an antibody, or an antigen-binding fragment thereof, comprising a VH according to SEQ ID NO: 72; and a VL according to SEQ ID NO: 73;
(v) an antibody, or an antigen-binding fragment thereof, comprising a VH according to SEQ ID NO: 80; and a VL according to SEQ ID NO: 81;
(vi) an antibody, or an antigen-binding fragment thereof, comprising a VH according to SEQ ID NO: 88; and a VL according to SEQ ID NO: 89.

78. The kit according to any one of claims 74 to 77, which comprises at least three antibodies, or antigen-binding fragments thereof, and preferably comprises an antibody, or an antigen-binding fragment thereof, according to (i), an antibody, or an antigen-binding fragment thereof, according to (ii), and an antibody, or an antigen-binding fragment thereof, according to at least one of (iii) to (vi).

79. The kit according to any one of claims 71 to 78, wherein the kit further comprises at least one additional agent useful for treating birch pollen allergy, and/or a related tree pollen allergy, which is preferably selected from at least one of hazel pollen allergy, black alder pollen allergy, beech pollen allergy, oak tree pollen allergy, chestnut pollen allergy, hop-hornbeam pollen allergy, and hornbeam pollen allergy, and/or a related food allergy, which is preferably selected from at least one of apple allergy, hazelnut allergy and walnut allergy.

80. The kit according to any one of claims 71 to 79, wherein the kit further comprises a birch pollen allergen, and/or a related tree pollen allergen, which is preferably selected from at least one of hazel pollen allergen, black alder pollen allergen, beech pollen allergen, oak tree pollen allergen, chestnut pollen allergen, hop-hornbeam pollen allergen, hornbeam pollen allergen, preferably selected from the group consisting of Cor a 1, Aln g 1, Fag s 1, Que a 1, Cas s 1, Ost c 1, Car b1, and/or a related food allergen, which is preferably selected from at least one of apple allergen, hazelnut allergen and walnut allergen, preferably selected from Mal d 1, Cor a 1 and Jug r 5.

81. The antibody, or an antigen-binding fragment thereof, according to any one of claims 1 to 49, the nucleic acid or the plurality of nucleic acids according to any one of claims 50 to 53, the vector or the plurality of vectors according to claim 54 or 55, the cell according to claim 56, the composition according to any one of claims 58 to 70, or the kit according to any one of claims 71 to 80 for use as a medicament.

82. The antibody, or an antigen-binding fragment thereof, according to any one of claims 1 to 49, the nucleic acid or the plurality of nucleic acids according to any one of claims 50 to 53, the vector or the plurality of vectors according to claim 54 or 55, the cell according to claim 56, the composition according to any one of claims 58 to 70, or the kit according to any one of claims 71 to 80 for use in the prophylaxis or treatment of a birch pollen allergy and/or related tree pollen allergy, which preferably selected from at least one of hazel pollen allergy, black alder pollen allergy, beech pollen allergy, oak tree pollen allergy, chestnut pollen allergy, hop-hornbeam pollen allergy and hornbeam pollen allergy, and/or a related food allergy, preferably selected from at least one of apple allergy, hazelnut allergy and walnut allergy.

83. The antibody, or the antigen-binding fragment thereof, the nucleic acid or the plurality of nucleic acids, the vector or the plurality of vectors, the cell, or the composition for use according to claim 82, wherein at least two or three distinct antibodies or antigen-binding fragments thereof, which bind to distinct non-overlapping epitopes of Bet v 1 and optionally bind to distinct non-overlapping epitopes of at least one Bet v 1 homologous protein; or nucleic acid(s) or vector(s) encoding the distinct antibodies; or composition(s) comprising the distinct antibodies are administered.

84. The antibody, or the antigen-binding fragment thereof, the nucleic acid or the plurality of nucleic acids, the vector or the plurality of vectors, the cell, or the composition for use according to claim 82 or 83, wherein the administration of the antibody, or the antigen-binding fragment thereof, the nucleic acid or the plurality of nucleic acids, the vector or the plurality of vectors, the cell, or the composition is combined with the administration of a Bet v 1 birch pollen allergen, and/or a related tree pollen allergen, preferably selected from at least one of Cor a 1 hazel pollen allergen, Ain g 1 black alder pollen allergen, Fag s 1 beech pollen allergen, Que a 1 oak tree pollen allergen, Cas s 1 chestnut pollen allergen, Ost c 1 hop-hornbeam pollen allergen and Car b1 hornbeam pollen allergen, and/or a related food allergen, preferably selected from at least one of Mal d 1 apple allergen, Cor a 1 hazelnut allergen and Jug r 5 walnut allergen.

85. The antibody, or the antigen-binding fragment thereof, or the composition for use according to claim 84, wherein the antibody, or the antigen-binding fragment thereof, or the composition is administered before or during a desensitization procedure with a birch pollen allergen, and/or a related tree pollen allergen, and/or a related food allergen.

86. Use of the antibody, or an antigen-binding fragment thereof, according to any one of claims 1 to 49, the composition according to any one of claims 58 to 70, or the kit according to any one of claims 71 to 80 in (in-vitro) diagnosis of a birch pollen allergy, and/or a related tree pollen allergy, preferably selected from at least one of hazel pollen allergy, black alder pollen allergy, beech pollen allergy, oak tree pollen allergy, chestnut pollen allergy, hop-hornbeam pollen allergy and hornbeam pollen allergy, and/or a related food allergy, preferably selected from at least one of apple allergy, hazelnut allergy and walnut allergy.

87. Use of the antibody, or an antigen-binding fragment thereof, according to any one of claims 1 to 49, the composition according to any one of claims 58 to 70, or the kit according to any one of claims 71 to 80 in a method for detecting a birch pollen allergen, and/or a related tree pollen allergen, preferably selected from at least one of Cor a 1 hazel pollen allergen, Ain g 1 black alder pollen allergen, Fag s 1 beech pollen allergen, Que a 1 oak tree pollen allergen, Cas s 1 chestnut pollen allergen, Ost c 1 hop-hornbeam pollen allergen and Car b1 hornbeam pollen allergen, and/or a related food allergen, preferably selected from at least one of Mal d 1 apple allergen, Cor a 1 hazelnut allergen and Jug r 5 walnut allergen.

88. Use of the antibody, or an antigen-binding fragment thereof, according to any one of claims 1 to 49, the nucleic acid or the plurality of nucleic acids according to any one of claims 50 to 53, the vector or the plurality of vectors according to claim 54 or 55, the cell according to claim 56, the composition according to any one of claims 58 to 70, or the kit according to any one of claims 71 to 80 in the manufacture of a medicament for prophylaxis, treatment or attenuation of a birch pollen allergy, and/or a related tree pollen allergy, preferably selected from at least one of hazel pollen allergy, black alder pollen allergy, beech pollen allergy, oak tree pollen allergy, chestnut pollen allergy, hop-hornbeam pollen allergy and hornbeam pollen allergy, and/or a related food allergy, preferably selected from at least one of apple allergy, hazelnut allergy and walnut allergy.

89. A method of treating, ameliorating or reducing a birch pollen allergy and/or a related tree pollen allergy, preferably selected from at least one of hazel pollen allergy, black alder pollen allergy, beech pollen allergy, oak tree pollen allergy, chestnut pollen allergy, hop-hornbeam pollen allergy and hornbeam pollen allergy, and/or a related food allergy, preferably selected from at least one of apple allergy, hazelnut allergy and walnut allergy, or lowering the risk of a allergic or anaphylactic reaction, comprising: administering to a subject in need thereof, a therapeutically effective amount of the antibody, or an antigen-binding fragment thereof, according to any one of claims 1 to 49, the nucleic acid or the plurality of nucleic acids according to any one of claims 50 to 53, the vector or the plurality of vectors according to claim 54 or 55, the cell according to claim 56, the composition according to any one of claims 58 to 70, or the kit according to any one of claims 71 to 80.
